(19) United States
(12) Patent Application Publication
(10) Pub. No.: US 2026/0176632 A1
(43) Pub. Date: Jun. 25, 2026

(54) CHEMICALLY MODIFIED ANTISENSE OLIGONUCLEOTIDES (ASOS) AND COMPOSITIONS FOR RNA EDITING

(71) Applicant: AIRNA Corporation, Cambridge, MA (US)

(72) Inventors: Tobias Merkle, Tübingen (DE); Konstantin Licht, Tübingen (DE)

(73) Assignee: AIRNA Corporation, Cambridge, MA (US)

(21) Appl. No.: 19/401,664

(22) Filed: Nov. 26, 2025

Related U.S. Application Data
(60) Provisional application No. 63/725,464, filed on Nov. 26, 2024.
(60) Provisional application No. 63/760,537, filed on Feb. 19, 2025.
(60) Provisional application No. 63/805,256, filed on May 13, 2025.

(51) Int. Cl. C12N 15/113; A61P 1/16

(52) U.S. Cl. CPC C12N 15/113; A61P 1/16; C12N 2310/11; C12N 2310/314; C12N 2310/315; C12N 2310/321; C12N 2310/322

(57) ABSTRACT

The present disclosure relates to a chemically modified oligonucleotide for use in site-directed A-to-I editing of a target RNA inside a cell with endogenous adenosine deaminase acting on RNA (ADAR), the oligonucleotide comprising a sequence capable of binding to a target sequence in a target RNA and a central base triplet (CBT) of 3 nucleotides (5′-N₊₁ N₀ N₋₁-3′), wherein N₀ is the central nucleotide directly opposite to a target adenosine in the target RNA that is to be edited, and wherein the oligonucleotide comprises an asymmetry of 25-1-8 in a 5′ to 3′ direction; and comprises: (i) one or more phosphorothioate (PS) linkages at one or more of position +22, position +21, position +11, and position +4; and/or (ii) a 2′-O-methyl (2′-O-Me) modified nucleosides at position +13 and/or position +9.

### RELATED APPLICATIONS

This application claims the benefit under 35 U.S.C § 119 (e) to U.S. Provisional Application No. 63/725,464, filed Nov. 26, 2024, entitled “CHEMICALLY MODIFIED ANTISENSE OLIGONUCLEOTIDES (ASOS) AND COMPOSITIONS FOR RNA EDITING,” U.S. Provisional Application No. 63/760,537, filed Feb. 19, 2025, entitled “CHEMICALLY MODIFIED ANTISENSE OLIGONUCLEOTIDES (ASOS) AND COMPOSITIONS FOR RNA EDITING,” and to U.S. Provisional Application No. 63/805,256, filed May 13, 2025, entitled “CHEMICALLY MODIFIED ANTISENSE OLIGONUCLEOTIDES (ASOS) AND COMPOSITIONS FOR RNA EDITING,” the contents of which are incorporated herein by reference in their entirety.

### REFERENCE TO AN ELECTRONIC SEQUENCE LISTING

The contents of the electronic sequence listing (A146270008US03-SUBSEQ-ZJG.xml; Size: 116,136 bytes; and Date of Creation: Dec. 18, 2025) are herein incorporated by reference in their entirety.

### TECHNICAL FIELD

The present disclosure relates to the field of site-directed RNA editing, whereby an RNA sequence is targeted by an antisense oligonucleotide (ASO) for RNA editing of a genetic mutation (“compensatory editing”) or for editing of an RNA derived from a wildtype allele (“beneficial editing”).

### BACKGROUND

RNA editing is a natural process through which some cells can make discrete changes to specific nucleotide sequences within an RNA molecule in a site-specific way. Unlike DNA editing, the advantage of site-directed RNA editing is that it allows modification of the genetic information that leads to a modified protein in a more precise, efficient, and safe manner. Contrary to DNA, RNA is generally quickly degraded and any errors introduced by off-target modifications to other RNAs will be washed out rather than permanently introduced into the modified DNA of a subject. RNA editing may also be less likely to cause an immune reaction since it is an editing mechanism naturally found in humans. Moreover, RNA editing might provide a more natural response than introducing an external, engineered gene.

Over the years, oligonucleotide therapeutics have been developed to silence, restore or modify the expression of disease-causing or disease-associated genes in, e.g., cancer and (other) genetic disorders. Such therapeutics include, e.g., antisense oligonucleotides (ASOs), small interfering RNA (siRNA) and microRNA (miRNA) that interfere with coding and noncoding RNAs in a sequence specific manner. The relative ease and accuracy with which ASOs can be customized allows virtually any gene to be targeted. As a result, ASOs are the most clinically developed, with several drugs already approved by the U.S. Food and Drug Administration (FDA) and in clinical trials (Cideciyan et al., 2019; Gagliardi and Ashizawa, 2021).

Site-Directed RNA Editing (SDRE) describes the alteration of an RNA sequence by introducing or removing nucleotides from an RNA or by changing the character of a nucleobase by deamination. RNA editing enzymes are known in the art. The first RNA editing process discovered in mammals was the deamination of cytidine (C) by APOBEC proteins to form uridine (U) (Zinshteyn and Nishikura, 2009). To date, the two most useful and most studied types of RNA editing are cytidine (C) to uridine (U) (“C-to-U”) and adenosine (A) to inosine (I) (“A-to-I”) conversions. Notably, for therapeutic purposes in higher eukaryotes the most prevalent type of RNA editing is the “A-to-I” conversion, which is catalysed by the adenosine deaminases acting on RNA (ADARs) family.

Over the years, three vertebrate ADAR genes have been identified, which give rise to several ADAR proteins through alternative promoters or splicing (Wulff and Nishikura, 2010). ADAR proteins are expressed across various types of human tissues and can alter, inter alia, splicing and translation machineries, double-stranded RNA (dsRNA) structures as well as the binding affinity between RNA and RNA-binding proteins (Tomaselli et al., 2014; Zinshteyn and Nishikura, 2009). Of the three known ADAR genes, hADAR1 and hADAR2 are expressed in most tissues and encode active deaminases. Human ADAR3 (hADAR3) has been described to only be expressed in the central nervous system and reportedly has no deaminase activity in vitro. While all ADARs are multidomain proteins, comprising a targeting or dsRNA-binding domain (dsRBD) and a catalytic domain, ADAR1 proteins additionally comprise one or more Z binding domains, while splice variant ADAR2R and ADAR3 comprises an R domain (Zinshteyn and Nishikura, 2009; Wulff and Nishikura, 2010). Accordingly, the ADAR may be hADAR1, hADAR2 or hADAR3, or any variant thereof. The ability of ADARs to alter the sequence of RNAs has also been used to artificially target RNAs in vitro in cells for RNA editing.

“A-to-I” editing was initially identified in Xenopus eggs (Bass and Weintraub, 1987; Rebagliati and Melton, 1987). Human cDNA encoding “double stranded RNA adenosine deaminase” was first cloned by Kim et al. (1994) and “A-to-I” conversion activity of the protein confirmed by recombinant expression in insect cells. Specifically, “A-to-I” editing changes the informational content of the RNA molecule, as inosine preferentially basepairs with cytidine and is therefore interpreted as guanosine (G) by the translational and splicing machinery. Therefore, ADARs have the effect of introducing a functional adenosine to guanosine mutation on the RNA level. Potentially, this approach may be used to repair genetic defects and alter genetic information at the RNA level.

### SUMMARY

In some aspects of the present disclosure, oligonucleotides (or antisense oligonucleotides, ASOs) with desirable properties for in vitro and in vivo use are provided. The problem solved by an invention provided herein lies in the provision of improved chemically modified ASOs capable of mediating a functional change from an adenosine (A) to a guanosine (G). In some embodiments, the invention relates to chemically modified oligonucleotides for use in site-directed A-to-I editing, comprising specific modifications as herein disclosed.

The solution to the technical problem is achieved by the embodiments described herein and defined by the appended claims.

The present disclosure generally provides for chemically modified oligonucleotides for use in site-directed A-to-I editing of a target RNA inside a cell with endogenous adenosine deaminase acting on RNA (ADAR).

In a first aspect provided herein is a chemically modified oligonucleotide for use in site-directed A-to-I editing of a target RNA inside a cell with endogenous adenosine deaminase acting on RNA (ADAR), the oligonucleotide comprising a sequence capable of binding to a target sequence in a target RNA and a central base triplet (CBT) of 3 nucleotides (5′-N₊₁ N₀ N₋₁-3′), wherein N₀ is the central nucleotide directly opposite to a target adenosine in the target RNA that is to be edited, and wherein the oligonucleotide comprises an asymmetry of 25-1-8 in a 5′ to 3′ direction; and comprises: (i) one or more phosphorothioate (PS) linkages at one or more of position +22, position +21, position +11, and position +4; and/or (ii) a 2′-O-methyl(2′-O-Me) modified nucleosides at position +13 and/or position +9.

In a second aspect, provides herein is a chemically modified oligonucleotide for use in site-directed A-to-I editing of a target RNA inside a cell with endogenous adenosine deaminase acting on RNA (ADAR), the oligonucleotide comprising a sequence capable of binding to a target sequence in a target RNA and a central base triplet (CBT) of 3 nucleotides (5′-N₊₁ N₀ N₋₁-3′), wherein N₀ is the central nucleotide directly opposite to a target adenosine in the target RNA that is to be edited, and wherein the oligonucleotide comprises the following core sequence: 5′- . . . N₊₅ N₊₄ N₊₃ N₊₂ N₊₁ N₀ N₋₁ N₋₂ N₋₃ N₋₄ N₋₅ . . . -3′, wherein the oligonucleotide comprises: (i) a phosphorothioate (PS) linkage at position +21 and position +4; (ii) a phosphodiester (PO) linkage at position +23 and position −5; and/or (iii) a sulfonylphosphoramidate internucleoside linkage at one or more positions selected from positions +24, +13, −2 and −8, wherein the sulfonylphosphoramidate internucleoside linkage is of formula (I):

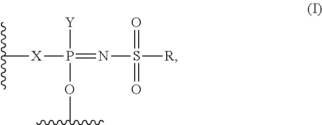

wherein X=Oxygen (O), providing a linkage to a sugar residue; R=a C₁-C₆ alkyl group or toluene group; and Y=O⁻ or —OH group.

In a third aspect provided herein is a chemically modified oligonucleotide, wherein the oligonucleotide comprises a nucleobase sequence of 5′-GCCCCAGCAGCTUCAGXCCCUUTCTCNUCGAUGG-3′ (SEQ ID NO: 1), wherein X=thymine (T) or uracil (U); wherein N=inosine (I) or guanosine (G); and wherein the oligonucleotide comprises: (i) a mesyl linkage, optionally at position +24, +13, −2, and/or −8; (ii) a PS linkage at position +21 and +4; and (iii) a PO linkage at position +23 and −5.

In a fourth aspect provided herein is a chemically modified oligonucleotide, wherein the oligonucleotide is selected from the group consisting of AI-2811 (SEQ ID NO: 5), AI-2814 (SEQ ID NO: 6), AI-2863 (SEQ ID NO: 7), AI-2864 (SEQ ID NO: 8), AI-2865 (SEQ ID NO: 9), AI-2866 (SEQ ID NO: 10), AI-3063 (SEQ ID NO: 11), compound (I), AI-4486 (SEQ ID NO: 21), AI-4487 (SEQ ID NO: 22), AI-4488 (SEQ ID NO: 23); or AI-4543 (SEQ ID NO: 24). In one embodiment, the chemically modified oligonucleotide is compound (I), i.e., AI-2863 (see, FIG. 8).

In a fifth aspect provided herein is a composition comprising the chemically modified oligonucleotide disclosed herein.

In a sixth aspect provided herein is a chemically modified oligonucleotide disclosed herein or composition disclosed herein for therapeutic use.

In a seventh aspect provided herein is a chemically modified oligonucleotide disclosed herein or a composition disclosed herein for use in the treatment of a disease or disorder, where in the disease or disorder is selected form the group consisting of liver, metabolic, neurodegenerative, and cardiac or cardiovascular diseases or disorders which can be treated with or is associated with a gain-of-function (GOF) or loss-of-function (LOF) mutation.

In an eighth aspect provided herein is a method for treating a subject suffering from a disease or disorder, comprising administering an effective amount of the chemically modified oligonucleotide disclosed herein or the composition disclosed herein to the subject.

In a ninth aspect provided herein is an in vitro method for site-directed A-to-I editing of a target RNA, the method comprising a step of contacting a cell comprising a target RNA with the chemically modified oligonucleotide disclosed herein or the composition disclosed herein.

### BRIEF DESCRIPTION OF DRAWINGS

The figures shown in the following are merely illustrative. The figures shall not be construed to limit any aspects of the present disclosure.

FIGS. 1A-1H represent graphs showing (FIG. 1A) the editing efficacy (%) of AI-2863 and AI-1068 and (FIG. 1B) M-AAT levels at day 7 ((FIG. 1A) and (FIG. 1B)), and (FIG. 1C) the editing efficacy (%) of AI-2863 and AI-1068 and (FIG. 1D) M-AAT levels at day 21. (FIG. 1E) and (FIG. 1F) Summary of results. (FIG. 1G) and (FIG. 1H) Tissue exposure of oligonucleotides at different concentrations.

FIGS. 2A-2C present bar graphs showing (FIG. 2A) target editing efficacy (%) in the liver (first study); (FIG. 2B) target editing efficacy (%) in the liver (repeat study); and (FIG. 2C) combination of studies.

FIGS. 3A-3E present bar graphs showing the (FIG. 3A) study outline, (FIG. 3B) ALT liver level, (FIG. 3C) AST liver level, (FIG. 3D) creatine kidney level, and (FIG. 3E) BUN kidney level.

FIGS. 4A-4F present graphs showing (FIG. 4A) the in vivo study outline; (FIG. 4B) TNF-α; (FIG. 4C) CCL2; (FIG. 4D) CXCL10; (FIG. 4E) IL-6; and (FIG. 4F) summary of cytokine profile.

FIGS. 5A-5C present bar graphs showing (FIG. 5A) cellular ATP levels; (FIG. 5B) cellular LDH release, and (FIG. 5C) caspase 3/7 activation levels.

FIGS. 6A-6F present graphs showing the levels of cytokine release from hPBMCs.

FIGS. 7A-7H show bar graphs showing (FIG. 7A) the percentage editing (%), (FIG. 7B) M-AAT levels, and (FIGS. 7C-7H) show a compilation of results obtained from different cohort studies.

FIG. 8 shows the atomic structure of oligonucleotide AI-2863 (compound (I)), including its nucleotide sequence and an N-acetylgalactosamine (GalNAc) moiety conjugated through a C7 linker.

FIGS. 9A-9D show graphs showing (FIG. 9A) the editing efficacy (%) of tested oligonucleotides at day 7; (FIG. 9B) the editing efficacy (%) of tested oligonucleotides at day 21; (FIG. 9C) M-AAT levels of tested oligonucleotides at day 7; and (FIG. 9D) M-AAT levels of tested oligonucleotides at day 21.

FIGS. 10A-10B show graphs depicting the tissue exposure of oligonucleotides at (FIG. 10A) 5 mg/kg and (FIG. 10B) 10 mg/kg by day 7 and day 21.

FIGS. 11A-11C show (FIG. 11A) the target editing efficacy (%) of the tested oligonucleotides; (FIG. 11B) oligonucleotide tissue exposure by day 7 and day 25, and (FIG. 11C) target editing efficacy (%) (repeat study).

FIGS. 12A-12D show bar graphs representing the (FIG. 12A) ALT liver level; (FIG. 12B) AST liver level; (FIG. 12C) creatine kidney level; and (FIG. 12D) BUN kidney level.

FIGS. 13A-13C present bar graphs showing the (FIG. 13A) TNF-α level, (FIG. 13B) IL-6 level, (FIG. 13C) spleen weight in response to the different lead oligonucleotides, and (FIG. 13D) summary of cytokine profile.

FIGS. 14A-14C present bar graphs showing (FIG. 14A) cellular ATP levels; (FIG. 14B) cellular LDH release, and (FIG. 14C) caspase 3/7 activation levels.

FIGS. 15A-15F present bar graphs showing the levels of TNF-α and IL-6 cytokine release from hPBMCs in the presence of lead oligonucleotides.

FIG. 16 shows the atomic structure of oligonucleotide AI-1550 (compound (II)), including its nucleotide sequence and an N-acetylgalactosamine (GalNAc) moiety conjugated through a C7 linker at its 3′ terminus.

FIG. 17 shows the atomic structure of oligonucleotide AI-2811 (compound (III)), including its nucleotide sequence and an N-acetylgalactosamine (GalNAc) moiety conjugated through a C7 linker at its 3′ terminus.

FIG. 18 shows the atomic structure of oligonucleotide AI-2814 (compound (IV)), including its nucleotide sequence and an N-acetylgalactosamine (GalNAc) moiety conjugated through a C7 linker at its 3′ terminus.

FIG. 19 shows the atomic structure of oligonucleotide AI-2864 (compound (V)), including its nucleotide sequence and an N-acetylgalactosamine (GalNAc) moiety conjugated through a C7 linker at its 3′ terminus.

FIG. 20 shows the atomic structure of oligonucleotide AI-2865 (compound (VI)), including its nucleotide sequence and an N-acetylgalactosamine (GalNAc) moiety conjugated through a C7 linker at its 3′ terminus.

FIG. 21 shows the atomic structure of oligonucleotide AI-2866 (compound (VII)), including its nucleotide sequence and an N-acetylgalactosamine (GalNAc) moiety conjugated through a C7 linker at its 3′ terminus.

FIG. 22 shows the atomic structure of oligonucleotide AI-2868 (compound (VIII)), including its nucleotide sequence and an N-acetylgalactosamine (GalNAc) moiety conjugated through a C7 linker at its 3′ terminus.

FIGS. 23A-23D present atomic structures of different internucleoside linkage modification types linked to the sugar residue of the nucleotide that forms the basis of the oligonucleotide backbone: (FIG. 23A) esyl linkage; (FIG. 23B) prosyl linkage; (FIG. 23C) busyl linkage; and (FIG. 23D) hesyl linkage, wherein X can be, e.g., —H or —OH.

FIG. 24 represents a graph showing the SERPINA1 target editing efficacy (%) of the different ASOs as a result of free uptake (gymnosis).

FIGS. 25A-25B show (FIG. 25A) the layout of the in vivo study design in mice and (FIG. 25B) a bar graph showing the SERPINA1 target editing efficacy (%) by day 7 in the mouse liver.

FIG. 26 shows the atomic structure of oligonucleotide AI-4486 (compound (IX)), including its nucleotide sequence and an N-acetylgalactosamine (GalNAc) moiety conjugated through a C7 linker at its 3′ terminus.

FIG. 27 shows the atomic structure of oligonucleotide AI-4487 (compound (X)), including its nucleotide sequence and an N-acetylgalactosamine (GalNAc) moiety conjugated through a C7 linker at its 3′ terminus.

FIG. 28 shows the atomic structure of oligonucleotide AI-4487 (compound (XI)), including its nucleotide sequence and an N-acetylgalactosamine (GalNAc) moiety conjugated through a C7 linker at its 3′ terminus.

FIG. 29 shows the atomic structure of oligonucleotide AI-4543 (compound (XII)), including its nucleotide sequence and an N-acetylgalactosamine (GalNAc) moiety conjugated through a C7 linker at its 3′ terminus.

FIG. 30 represents a bar graph showing the editing efficiency (%) of AI-0991 at varying concentrations (100 nm-1 nM) provided alone or with interferon (IFN).

FIGS. 31A-31B present line graphs showing (FIG. 31A) SERPINA1 mRNA editing efficiency (%) in mice treated with two doses of AI-0991 at 1 mg/kg or 2 mg/kg) and (FIG. 31B) M-AAT concentration (μM) for the same doses over time. Measurements were taken at day 1, day 4, and day 7.

FIGS. 32A-32G show (FIG. 32A) the layout of the in vivo study design in mice, (FIG. 32B) liver oligonucleotide content measured by an MSD hybridization assay on Day 7 and Day 56 Lower Limit of Quantification (LLOQ)=0.49 μg ASO per g tissue), (FIG. 32C) liver RNA editing levels on Day 7 and Day 56. Statistical significance of differences between ASO-treated groups and the vehicle group was determined by ordinary one-way ANOVA with Bonferroni's multiple comparisons test. ****P<0.0001, (FIG. 32D) and (FIG. 32E) Time course of M-AAT (FIG. 32D) and total AAT (FIG. 32E) serum levels, measured by LC-MS/MS (LLOQ=0.03 μmol/L). Dashed line indicates the optimal level of 14 μmol/L M-AAT. Statistical significance of differences between ASO-treated groups and the vehicle control was determined by two-way ANOVA with Bonferroni's multiple comparisons test. *P<0.05; **P<0.01; ***P<0.001; ****P<0.0001; ns, not significant, (FIG. 32F) summary of terminal serum levels of M-AAT (solid bars) and total AAT (patterned bars), measured by LC-MS/MS (LLOQ=0.03 μmol/L). Dashed line indicates the protective threshold of 14 μmol/L M-AAT. Statistical significance of differences between ASO-treated groups and the age-matched vehicle group was determined by ordinary one-way ANOVA with Bonferroni's multiple comparisons test. Same level of significance (P<0.0001) was determined for M-AAT and total AAT, and (FIG. 32G) functional AAT levels on Day 7 and 56, determined by a neutrophil elastase inhibition assay. LLOQ was 2.1 μmol/L. Values below quantification limit were set to LLOQ for plotting and statistical analysis. Statistical significance of differences between ASO-treated groups and the age-matched vehicle group was determined by ordinary one-way ANOVA with Bonferroni's multiple comparisons test. ****P<0.0001; **P=0.0027; ns, not significant.

FIGS. 33A-33D present bar graphs showing (FIG. 33A) density of PAS-D-stained large Z-AAT globules (>60 μm2) in fixed livers following vehicle or AI-1068 treatment. Livers from Day-56 necropsy were evaluated here and below. Statistical significance of differences between ASO-treated groups and the vehicle group was determined by ordinary one-way ANOVA with Bonferroni's multiple comparisons test. *P=0.023; **P=0.001, (FIG. 33B) percentage of Ki-67-positive cells in fixed livers following vehicle or AI-1068 treatment. Statistical significance of differences between ASO-treated groups and the vehicle group was determined by ordinary one-way ANOVA with Bonferroni's multiple comparisons test. *P=0.0185; **P=0.0071, (FIG. 33C) inflammatory infiltrate scores (pleocellular, portal/periportal). Microscopic changes were graded as: 0=no significant change, 1=minimal, 2=mild, 3=moderate, and 4=marked change. Statistical significance of differences between ASO-treated groups and the vehicle group was determined by ordinary one-way ANOVA with Bonferroni's multiple comparisons test. *P=0.0304; ns, not significant, and (FIG. 33D) inflammatory infiltrates scores (pleocellular, parenchymal). Microscopic changes were graded as described in FIG. 33C. Statistical significance could not be evaluated because of zero variance in all groups.

FIG. 34 shows SERPINA 1 editing levels. Primary human hepatocytes with MZ genotype were plated and treated with AI-2863 at varying concentrations. Average and standard deviation of three replicate measurements are shown. Baseline editing level is 50% due to MZ genotype.

FIGS. 35A-35F show the effects of AI-2863 in NSG-PiZ mice treated subcutaneously with varying concentrations of AI-2863. FIG. 35A shows the study design. 7 week old male and female NSG PiZ mice were dosed subcutaneously with DPBS (vehicle) or 1 10 mg/kg AI-2863 on Days 0, 2, and 4. Terminal collection of liver and serum was performed on Days 7, 14, 21, 28, and 35. Collection of in-life serum (asterisks) was performed before the first dose and at 0.5, 2, 6, 24 and 48 hours after the last dose. Mean±SD, n=6 mice per group. FIG. 35B shows a time course of A to I editing levels at the site of the PiZ mutation, determined by amplicon sequencing. Statistical significance of the differences between ASO-treated groups and the control group on each necropsy day was determined by a one-way ANOVA with Bonferroni's multiple comparisons test. *P<0.05; **P<0.01; ***P<0.001; ****P<0.0001; ns, not significant. FIGS. 35C-35D show serum levels of wildtype (FIG. 35C) and total (FIG. 35D) human AAT protein, measured by LC-MS/MS (LLOQ=0.12 μmol/L). Statistical significance of the differences between ASO-treated groups and the control group on each necropsy day was determined by a one-way ANOVA with Bonferroni's multiple comparisons test. *P<0.05; **P<0.01; ***P<0.001; ****P<0.0001; ns, not significant. FIG. 35E shows the average number of liver inflammatory foci across 5 fields. Inflammation was evaluated in preserved livers from mice treated with vehicle (DPBS) or 10 mg/kg AI-2863 necropsied on Days 7 and 28. Statistical significance was evaluated by an unpaired t-test. ***P=0.0003; ns, not significant. FIG. 35F shows the mean density of large PAS-D-stained globules in the liver on Day 28. Globule density was evaluated in preserved, PAS-D-stained livers from mice treated with vehicle (DPBS) or 10 mg/kg AI-2863 and is presented as mean density relative to the vehicle control. Statistical significance was evaluated by an unpaired t-test. *P<0.05.

FIGS. 36A-36G show the effects of bi-weekly treatment with varying doses of AI-2863 in NSG-PiZ mice. FIG. 36A shows the study design. 7 week old male and female NSG PiZ mice were dosed subcutaneously with DPBS or 2.5 10 mg/kg AI-2863 on Days 0, 2, and 4, followed by dosing every 2 weeks (magenta arrows). In-life serum collection was performed weekly. Terminal liver and serum collection was performed on Day 106. FIG. 36B shows RNA editing levels at the site of the PiZ mutation, determined by amplicon sequencing in livers collected on Day 106 (n=3 males per group; mean±SD). Statistical significance of differences between AI-2863-treated groups and the control group was determined by one-way ANOVA with Bonferroni's multiple comparisons test. ****P<0.0001. FIG. 36C shows serum AAT levels on Day 7 and Day 106, measured by LC-MS/MS (LLOQ=0.12 μmol/L). Solid bars indicate M-AAT levels, and patterned bars indicate total AAT levels. Statistical significance of differences between AI-2863-treated groups and the control group was determined by two-way ANOVA with Bonferroni's multiple comparisons test. **P<0.01; ***P<0.001; ****P<0.0001; ns, not significant. FIG. 36D shows serum levels of M-AAT, measured by LC-MS/MS. Statistical significance of differences between ASO-treated groups and vehicle group was determined by two-way ANOVA with Bonferroni's multiple comparisons test. *P<0.05; **P<0.01; ***P<0.001; ****P<0.0001. FIG. 36E shows serum levels of total AAT, measured by LC-MS/MS. Statistical significance of differences between ASO-treated groups and vehicle group was determined by two-way ANOVA with Bonferroni's multiple comparisons test. *P<0.05; **P<0.01; ***P<0.001; ****P<0.0001; ns, not significant. FIG. 36F shows serum levels of functional AAT on Day 106, quantified by a neutrophil elastase inhibition assay (LLOQ=1.95 μmol/L). Dashed horizontal line indicates the LLOQ. Statistical significance of differences between AI-2863-treated groups and the control group was determined by one-way ANOVA with Bonferroni's multiple comparisons test. *P=0.0188; ****P<0.0001; ns, not significant. FIG. 36G shows the correlation between serum levels of M-AAT (quantified by LC-MS/MS) and functional AAT (quantified by neutrophil elastase assay) on Day 106. R2=0.97, slope=0.99. Solid line indicates the line of identity. Dashed horizontal line indicates the LLOQ of the neutrophil elastase assay.

FIGS. 37A-37D show editing levels and serum AAT levels in NSG-PiZ mice treated with a single dose of AI-2863. FIG. 37A shows the study design. 7-week-old male NSG-PiZ mice were dosed subcutaneously with DPBS or 10-100 mg/kg AI-2863. Terminal tissue and serum collection was performed on Day 7. n=9 males per group. FIG. 37B shows liver RNA editing levels at the site of the PiZ mutation, determined by amplicon sequencing. Statistical significance of differences between AI-2863-treated groups and the vehicle group was determined by one-way ANOVA with Bonferroni's multiple comparisons test. ****P<0.0001. FIGS. 37C-37D show serum levels of wildtype (FIG. 37C) and total (FIG. 37D) human AAT protein, measured by LC-MS/MS (LLOQ=0.12 μmol/L). Dashed horizontal line indicates the optimal level of 14 μmol/L M-AAT. Statistical significance of differences between ASO-treated groups and the vehicle group was determined by one-way ANOVA with Bonferroni's multiple comparisons test. ****P<0.0001.

### DETAILED DESCRIPTION

### Terminology

In order that any one of the inventions disclosed herein may be more readily understood, certain terms are first defined.

The articles “a” and “an” are used herein to refer to one or to more than one (i.e., to at least one) of the grammatical object of the article. By way of example, “an element” means one element or more than one element, e.g., a plurality of elements.

The terms “about” and “approximately” may be understood to permit standard variation as would be understood by those of ordinary skill in the art.

The term “including” is used herein to mean, and is used interchangeably with, the phrase “including, but not limited to”. Likewise, the term “comprising” is used herein to mean, and is used interchangeably with, the phrase “comprising, but not limited to”.

As used herein a “sulfonylphosphoramidate internucleoside linkage” generally is or consists of formula (I):

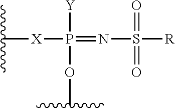

wherein X=Oxygen (O), providing a linkage to, e.g., the 5′ carbon of the sugar residue; R=a C₁-C₆ alkyl group; and Y=O⁻ or —OH group.

As used herein, the expression “phosphorothioate linkage” or “PS linkage” refers to a modification of the phosphate backbone of an oligonucleotide, wherein the phosphodiester (PO) linkage (C—O—PO-2O—C) between the 5′ carbon of one nucleoside and the 3′ carbon of the adjacent nucleoside is modified, i.e., replaced, by a phosphorothioate (PS) linkage.

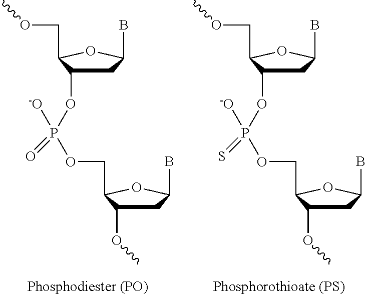

As used herein, the expressions “mesyl phosphoramidate”, “methanesulfonyl phosphoramidate” and “methanesulfonyl (mesyl)” internucleoside linkage (modification) have the same meaning and can be used interchangeably. As used herein, a mesyl linkage is denoted by the symbol “&”. The oligonucleotides disclosed herein contain at least one mesyl (&) linkage, which means that at least one mesyl phosphoramidate linkage is incorporated into the ASO backbone instead of the natural phosphate (PO) linkage (i.e., phosphodiester group).

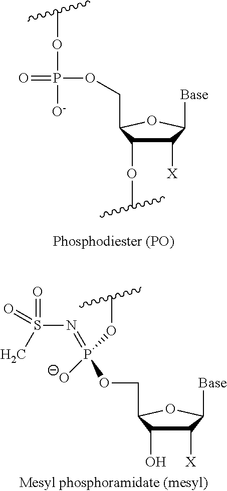

As used herein, a “mesyl” linkage at position +24 (N₊₂₄) indicates that the nucleotide at position 24 is linked via its phosphate to a (NSO₂CH₃) group and that the mesyl linkage is located between nucleotide 25 (N₊₂₅) and nucleotide 24 (N₊₂₄). Likewise, if it is indicated the mesyl position is located at “−8” (N₋₈), this means that the mesyl linkage is between the nucleotides located at position −7 (N₋₇) and −8 (N₋₈). As used herein, nucleotide positions that are underlined indicate the terminal or penultimate positions containing a mesyl linkage, e.g., mesyl pattern “+24, +13, −2, −8”. Depending on whether it is DNA or RNA, “X” can be H or OH respectively. As used herein, the term “ethanesulfonyl phosphoramidate”, “ethanesulfonyl” or “esyl” internucleoside linkage (modification) have the same meaning and can be used interchangeably. It refers to a modified phosphodiester bond, that has been modified such that the natural phosphate (PO) linkage (i.e., phosphodiester group) in the oligonucleotide backbone has been modified such that the phosphate forms an internucleoside linkage with an ethanesulfonamide group (NSO₂CH₂CH₃). Depending on whether it is DNA or RNA, “X” is H or OH respectively.

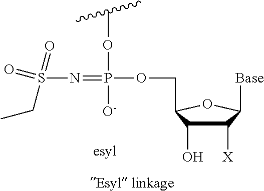

The oligonucleotides disclosed herein may contain at least one “esyl” linkage, which means that at least one “esyl” linkage is incorporated into the ASO backbone instead of the natural phosphate (PO) linkage (i.e., phosphodiester group). For instance, as used herein, a “esyl” linkage at position +24 (N₊₂₄) indicates that the nucleotide at position 24 is linked via its phosphate to a (N₂SO₂CH₂CH₃) group and that the esyl linkage is located between nucleotide 25 (N₊₂₅) and nucleotide 24 (N₊₂₄).

As used herein, the term “prosyl phosphoramidate”, “1-propanesulfonyl” or “prosyl” internucleoside linkage (modification) have the same meaning and can be used interchangeably. It refers to a modified phosphodiester bond, that has been modified such that the natural phosphate (PO) linkage (i.e., phosphodiester group) in the oligonucleotide backbone has been modified such that the phosphate forms an internucleoside linkage with a propane-1-sulfonamide group (NSO₂CH₂CH₂CH₃). Depending on whether it is DNA or RNA, “X” is H or OH respectively.

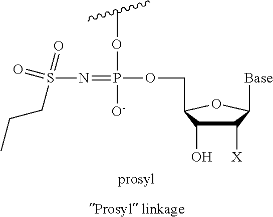

The oligonucleotides disclosed herein may contain at least one “prosyl” linkage, which means that at least one “prosyl” linkage is incorporated into the ASO backbone instead of the natural phosphate (PO) linkage (i.e., phosphodiester group). For instance, as used herein, a “prosyl” linkage at position +24 (N₊₂₄) indicates that the nucleotide at position 24 is linked via its phosphate to a (NSO₂CH₂CH₂CH₃) group and that the prosyl linkage is located between nucleotide 25 (N₊₂₅) and nucleotide 24 (N₊₂₄).

As used herein, the term “busyl phosphoramidate”, “1-butanesulfonyl” or “busyl” internucleoside linkage (modification) have the same meaning and can be used interchangeably. It refers to a modified phosphodiester bond, that has been modified such that the natural phosphate (PO) linkage (i.e., phosphodiester group) in the oligonucleotide backbone has been modified such that the phosphate forms an internucleoside linkage with a butane-1-sulfonamide group (NSO₂CH₂CH₂CH₂CH₃). Depending on whether it is DNA or RNA, “X” is H or OH respectively.

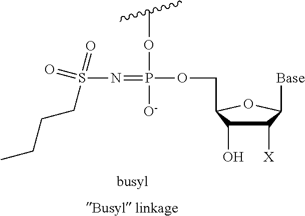

The oligonucleotides disclosed herein may contain at least one “busyl” linkage, which means that at least one “busyl” linkage is incorporated into the ASO backbone instead of the natural phosphate (PO) linkage (i.e., phosphodiester group). For instance, as used herein, a “busyl” linkage at position +24 (N₊₂₄) indicates that the nucleotide at position 24 is linked via its phosphate to a (NSO₂CH₂CH₂CH₂CH₃) group and that the busyl linkage is located between nucleotide 25 (N₊₂₅) and nucleotide 24 (N₊₂₄).

As used herein, the term “hexyl phosphoramidate”, “1-hexanesulfonyl”, “hexyl” or “hesyl” internucleoside linkage (modification) have the same meaning and can be used interchangeably. It refers to a modified phosphodiester bond, that has been modified such that the natural phosphate (PO) linkage (i.e., phosphodiester group) in the oligonucleotide backbone has been modified such that the phosphate forms an internucleoside linkage with a hexane-1-sulfonamide group (NSO₂CH₂CH₂CH₂CH₂CH₂CH₃). Depending on whether it is DNA or RNA, “X” is H or OH respectively.

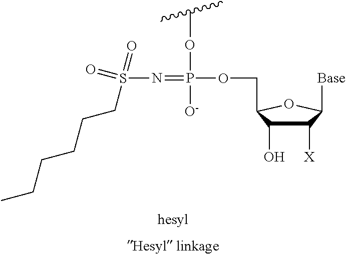

The oligonucleotides disclosed herein may contain at least one “hesyl” linkage, which means that at least one “hesyl” linkage is incorporated into the ASO backbone instead of the natural phosphate (PO) linkage (i.e., phosphodiester group). For instance, as used herein, a “hesyl” linkage at position +24 (N₊₂₄) indicates that the nucleotide at position 24 is linked via its phosphate to a (N₂SO₂CH₂CH₂CH₂CH₂CH₂CH₃) group and that the hesyl linkage is located between nucleotide 25 (N₊₂₅) and nucleotide 24 (N₊₂₄).

As used herein, the terms “toluenesulfonyl”, “p-toluenesulfonyl” and “tosyl” internucleoside linkage (modification) have the same meaning and may be used interchangeably. The tosyl group may be derived from p-toluenesulfonic acid (CH₃C₆H₄SO₃H). The oligonucleotides disclosed herein may contain at least one tosyl linkage, which means that at least one tosyl linkage is incorporated into the ASO backbone instead of the natural phosphate (PO) linkage (i.e., phosphodiester group). This means that nucleotides that are “tosylated” carry at CH₃C₆H₄SO₂ group linked via a nitrogen to the phosphate between the 3′ carbon atom of the sugar molecule and the 5′ carbon atom of the neighbouring sugar molecule.

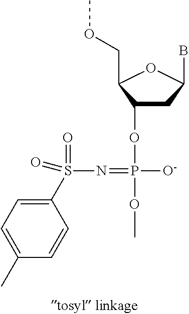

As used herein the term “flanking region” refers to the 5′ and/or 3′ region on the oligonucleotide is adjacent or directly adjacent to the N₀ on the 5′ and/or 3′ portion of the oligonucleotide. In one embodiment, the flanking region is located directly adjacent to N₀. Alternatively, in one embodiment, a flanking region is located anywhere upstream and/or anywhere downstream of N₀. In one embodiment, the flanking region is located at the far end of the 5′ terminus and/or at the far end of the 3′ terminus. The flanking region may comprise one or more nucleotide, i.e., a range of nucleotides. For instance, the flanking region my comprise 1, 2, 3, 4, 5, 6, 7, 8, 9 or more nucleotides 5′ and/or 3′ to N₀. That is, in some embodiments, the flanking region comprises the entire region 5′ and/or 3′ to N₀, in other embodiments the flanking region comprises the outermost 1, 2, or 3 nucleotides at the 5′ and/or 3′ terminus.

As used herein, the term “nucleic acid” is intended to include any DNA molecule (e.g., cDNA or genomic DNA) and any RNA molecule (e.g., mRNA) and analogues of the DNA or RNA generated using nucleotide analogues. Oligonucleotides can be single-stranded (ss) or double-stranded (ds). A single-stranded oligonucleotide can have double-stranded regions (formed by portions of the single-stranded oligonucleotide). A double-stranded oligonucleotide can have single-stranded regions, for example, at regions where the two oligonucleotide chains are not complementary to each other. Each component of the DNA or RNA can be modified and categorized by modification of (1) the internucleoside linkage, (2) the deoxyribose/ribose, and/or (3) the nucleobase.

The term “nucleobase” or “base” refers to biological building blocks that can form nucleosides, which, in turn, may be components of nucleotides. Naturally occurring bases are generally guanine, (G), adenine, (A), cytosine, (C), thymine, (T), and uracil (U), which are derivatives of purine or pyrimidine. Cytosine, thymine, and uracil are pyrimidine bases that are generally linked to the backbone through their 1-nitrogen. Adenine and guanine are purine bases and generally linked to the backbone through their 9-nitrogen. It should be understood that naturally and non-naturally occurring base analogues are also included and that the term “nucleobase” also includes “modified nucleobases”.

Within the context of this disclosure, the term “modified nucleobase” and “modified base” may be used interchangeably with the term “nucleobase”. A nucleobase may be a nucleobase, which comprises a modification. In some embodiments, a modified nucleobase is capable of at least one function of a nucleobase, e.g., forming a moiety in a polymer capable of base-pairing to a nucleic acid comprising an at least complementary sequence of bases. In one embodiment, the modified nucleobase is capable of increasing hydrogen bonding, base pair stacking interactions and/or stabilizing a nucleic acid complex. The modified nucleobase (e.g., Benner's base) may be capable of mimicking the N3 protonated cytosine base. In some embodiments, a modified nucleobase is substituted A, T, C, G, or U, or a substituted tautomer of A, T, C, G, or U. In some embodiments, a modified nucleobase in the context of oligonucleotides refer to a nucleobase that is not A, T, C, G or U. Modifications include but are not limited to nonstandard nucleobases 5-methyl-2′-deoxycytidine (m⁵C), pseudouridine (pU), dihydrouridine, inosine (I), and 7-methylguanosine. In some embodiments, the modification is iso-uridine (SbU). Other modifications may include nucleobase replacement by (N) heterocycles (e.g., nebularine) or aromatic rings that stack well in the RNA duplex, such as, e.g., a Benner's base Z (and/or analogues) or 8-oxo-adenosine (8-oxo-A). As used herein, the term “Benner's base Z” refers to the pyrimidine analogue 6-amino-5-nitro-3-(1′-β-D-2′-deoxyribofuranosyl)-2 (1H)-pyridone (dZ). In one embodiment, a modification includes the introduction of nucleobase analogues or simple heterocycles that boost editing. As used herein, and as commonly understood by the skilled person in the art, the expression “derivative thereof” refers to a derivative of a (modified) nucleobase, nucleoside or nucleotide. For example, a derivative may be a corresponding nucleobase, nucleoside or nucleotide that has been chemically derived from said nucleobase, nucleoside or nucleotide. For instance, a derivative of deoxycytidine may include fluoro-modified deoxycytidine, 5-methyl-2′-deoxycytidine (m⁵C), or ribocytidine.

The term “nucleoside(s)” refers to a moiety wherein a nucleobase or a modified nucleobase is covalently bound to a sugar or a modified sugar. In some embodiments, a “nucleoside” refers to a nucleoside unit in an oligonucleotide or a nucleic acid. The term “nucleoside(s)” encompasses all modified versions and derivatives “modified nucleobases”.

The term “nucleotide(s)” as used herein refers to a monomeric unit of a polynucleotide that consists of a nucleobase, a sugar, and one or more linkages (e.g., phosphate linkages in natural DNA and RNA). In some cases, the linkage may be a non-naturally occurring and/or modified linkage. In some embodiments, the linkage may be an internucleoside linkage as described herein. In one specific embodiment, the modified linkage is a PS linkage. In some embodiments, a “nucleotide” refers to a nucleotide unit in an oligonucleotide or a nucleic acid. The term “nucleotide(s)” encompasses all modified versions and derivatives of “nucleosides” and “modified nucleobases”.

The term “oligonucleotide(s)” as used herein is defined as is generally understood by the skilled person as a molecule including two or more covalently linked nucleosides. They can comprise DNA and/or RNA. The oligonucleotides may have a backbone comprising deoxyribonucleotides and/or ribonucleotides.

The term “internucleoside linkage” refers to a linkage between adjacent nucleosides. “Internucleoside linkage” and “linkage” may be used interchangeably. Linkages may be continuous (consecutive) or discontinuous (interrupted). As used herein, the term “discontinuous” or “interrupted” means that there are not more than, e.g., 4, 5, 6, 7 or more consecutive internucleoside linkages of the same linkage. In some embodiments, the naturally occurring PO linkages are replaced by modified internucleoside linkages. Hence, in some embodiments, the linkage is a non-natural internucleoside linkage.

As used herein the term “stereopure” or “stereorandom” refers to chemically modified oligonucleotides. Specifically, the term “stereopure” refers to oligonucleotides that are chirally pure (or “stereochemically pure”). The term “stereorandom” refers to racemic (or “stereorandom”, “non-chirally controlled”) oligonucleotides. Hence, the oligonucleotides disclosed herein comprise 1, 2, 3, 4, 5, 6, 7, 8, 9, 10 or more stereorandom internucleoside linkages (mixture of Rp and Sp linkage phosphorus at the internucleoside linkage, e.g., from traditional non-chirally controlled oligonucleotide synthesis). In one embodiment, an internucleoside linkage is a phosphorothioate (PS) linkage. In one embodiment, an internucleoside linkage is a stereorandom PS linkage. In one embodiment, an internucleoside linkage is a chirally controlled PS linkage. In one embodiment, an internucleoside linkage is not chirally controlled. In one embodiment, an internucleoside linkage is not a chirally controlled PS linkage.

As used herein the term “antisense oligonucleotide” or “ASO” refers to a strand of nucleotide analogue that hybridizes with the complementary (target) RNA in a sequence-specific manner via Watson-Crick base pairing. The ASO may be chemically modified. The terms “antisense oligonucleotide” and “oligonucleotide” may be used interchangeably.

As used herein, the term “target RNA” refers to an RNA, which is subject to the editing process, and “targeted” by the respective ASOs disclosed herein.

As used herein, the term “off-target” or “off-targeting” refers to non-specific and/or unintended genetic modification(s) of the target. Off-target editing may include unintended point mutations, deletions, insertions, inversions, and translocations. For instance, off-target editing may arise from the promiscuous reactivity of the deaminase enzymes.

The term “modified sugar” refers to a moiety that can replace a naturally occurring sugar. A modified sugar may mimic the spatial arrangement, electronic properties, or some other physicochemical property of a sugar. The naturally occurring sugar is generally the pentose deoxyribose or ribose, though it should be understood that naturally and non-naturally occurring sugar analogues are also included. For example, sugars may comprise C4 sugars, C5 sugars and/or C6 sugars. In some embodiments, a modified sugar is substituted. In some embodiments, a modified sugar is a sugar that is not ribose or deoxyribose as typically found in natural RNA or DNA (e.g., arabinose). In some embodiments, a modified sugar comprises a 2′-modification. Examples of useful 2′-sugar modifications include, e.g., 2′-ribose (RNA), 2′-deoxyribose (DNA), 2′-arabinose etc., Those skilled in the art, will appreciate that various types of 2′-sugar modifications are known that can be used in accordance with the present disclosure. In one embodiment, the 2′-sugar modification is 2′-ribose. In one embodiment, the 2′-sugar modification is 2′-deoxyribose. The term “locked nucleic acid” (LNA) or “locked nucleic acids” (LNAs) are also known as bridged nucleic acid (BNA) and refers to modified RNA nucleotide in which the ribose moiety is modified with an extra bridge connecting the 2′ oxygen and 4′ carbon. In some embodiments, a modified sugar is a bicyclic sugar, e.g., a sugar used in locked nucleic acid (LNA), BNA, etc., In some embodiments, a modified sugar is an LNA sugar. In some embodiments, a modified sugar is an BNA sugar. In some embodiments, a sugar modification is 2′-OMe, 2′-O-methoxyethyl(2′-MOE), 2′-F, 5′-vinyl, or S-constrained ethyl (S-cEt). In one embodiment, a 2′-modification is a C2-stereoisomer of 2′-F-ribose. In one embodiment, a 2′-modification is 2′-F. In one embodiment, a 2′-modification is 2′-FANA. In one embodiment, a modified sugar is a sugar of morpholino. In one embodiment, the oligonucleotide comprises, e.g., an UNA (unlocked nucleic acid), a PMO (phosphorodiamidate linked morpholino) or a PNA (peptide nucleic acid). In one embodiment, the nucleic acid analogue is a PNA (peptide nucleic acid). In one embodiment, the nucleic acid analogue is PMO (phosphorodiamidate linked morpholino).

The term “FANA” or “FANA-modified” refers to 2′-fluoroarabinoside modified nucleobases and/or oligonucleotides comprising such nucleobases. For example, the expression “FANA-cytidine” refers to a cytidine that comprises a 2′-fluoro-beta-D-arabinonucleic acid sugar modification. Within the context of this disclosure, the expression “a derivate thereof” refers to a corresponding nucleotide(s) or oligonucleotide(s) that has been chemically derived from said nucleotide or oligonucleotide(s).

As used herein, the term “complementary”, “partially complementary” or “substantially complementary” refer to nucleic acid sequences, which, due to their complementary nucleotides, are capable of specific intermolecular base-pairing. The oligonucleotide may comprise a nucleic acid sequence complementary to a target sequence, e.g., SERPINA1, or any other target sequence. The ASO may be self-complementary. The ASO may be complementary to a coding or non-coding sequence. As those skilled in the art appreciate, perfect (e.g., 100%) complementarity or pairing is not required and one or more wobbles (wobble base pairing), bulges, mismatches, etc. may be tolerated. The one or more wobbles, bulges, mismatches, etc. may be within or outside the CBT. Hence, in one embodiment, the ASOs comprise a wobble base outside the CBT. In one embodiment, the ASO comprises a mismatch outside the CBT. For example, the ASOs may include a mismatch opposite the target adenosine. Hence, the complementarity of the ASOs may be 100%, except at the nucleoside opposite to a target nucleoside to be edited. In one embodiment, complementarity is at least 80%, 85%, 90%, 95%. In one embodiment, complementarity is 85%-99%. In one embodiment, the ASO comprises 1, 2, 3, 4, 5 or more mismatches when aligned with the target nucleic acid. In one embodiment, one or more mismatches are independently a wobble base paring. In one embodiment, the ASOs comprise up to 4 mismatches or wobble bases outside the CBT. In one embodiment, the ASOs comprise up to 3 mismatches or wobble bases outside the CBT.

The term “mutation” as used herein, refers to a substitution of a residue with another residue within a sequence, e.g., a nucleic acid sequence or amino acid sequence, or to a deletion or insertion of one or more residues within a sequence, e.g., point mutation. Mutations are typically described herein by identifying the original residue followed by the position of the residue within the sequence and by the identity of the newly substituted residue. Notably, the methods described herein are not limited to correcting mutations, as it may instead be useful to change a wildtype sequence into a mutated sequence using the ASOs disclosed herein. Various methods for making amino acid substitutions are well known in the art, and are provided by, e.g., Green and Sambrook, Molecular Cloning: A Laboratory Manual (4th ed., Cold Spring Harbor Laboratory Press, Cold Spring Harbor, N.Y. (2012)).

As used herein, the term “beneficial editing” refers to the editing of a target sequence (or base) derived from a wildtype allele (not a mutated allele) in order to, e.g., modulate the function of a wildtype protein in a useful way to prevent or treat a disease. For example, beneficial editing may include sites, such as STAT1 Y701, NLRP3 Y166 and CTNNB1 T41 that are not causes for genetic diseases but rather represent wildtype protein sites. These sites may be changed (no underlying G-to-A mutation) to alter the function of the wildtype protein.

The term “compensatory editing” refers to the modification of RNA nucleotides to change and correct one or more detrimental or unfavourable changes in the RNA sequence when compared to wildtype, e.g., a compensatory A-to-I change could help to functionally compensate for an otherwise non-editable mutation to ameliorate a disease phenotype.

The term “adenosine deaminase(s)” or “adenosine deaminase(s) acting on RNA” [ADAR(s)], as used herein, refers to any (poly) peptide, protein or protein domain or fragment thereof capable of catalysing the hydrolytic deamination of adenosine to inosine. The term thus not only refers to full-length and wild type ADARs but also to a functional fragment or a functional variant of an ADAR. In some embodiments, the ADAR is an (endogenous) adenosine deaminase catalysing the deamination of adenosine to inosine or deoxy-adenosine to deoxyinosine. In some embodiments, the ADAR catalyses the deamination of adenine or adenosine in deoxyribonucleic acid (DNA) or in ribonucleic acid (RNA). The ADAR may be a human ADAR. The ADAR may be an endogenous ADAR. Accordingly, in some embodiments, the ADAR is an endogenous human ADAR1, ADAR2 or ADAR3 (hADAR1, hADAR2 or hADAR3), or any fragment or isoform(s) thereof (e.g., hADAR1 p110 and p150).

The term “guide RNA” (gRNA) or “guide oligonucleotide” refers to a piece of RNA or oligonucleotide (comprising RNA and/or DNA) that functions as a guide for enzymes, with which it forms complexes. The guide RNA or guide oligonucleotide may comprise endogenous and/or exogenous sequences. Guide RNAs bind to their target in a sequence-specific manner. Guides can be used in vitro and in vivo. For example, the guide RNA or guide oligonucleotide directs the base-modifying activity/editing function (e.g., ADAR) to the target to be edited in trans.

As used herein, the terms “disease” or “disorder” are used interchangeably to refer to a condition in a subject. In certain embodiments, the condition is a disease in a subject, the severity of which is decreased by inducing an immune response in the subject through the administration of a pharmaceutical composition.

As used herein, the term “effective amount” in the context of administering a therapy to a subject refers to the amount of a therapy which has a prophylactic and/or therapeutic effect(s).

As used herein, the term “in combination” in the context of the administration of two or more therapies to a subject, refers to the use of more than one therapy (e.g., more than one prophylactic agent and/or therapeutic agent). The use of the term “in combination” does not restrict the order in which therapies are administered to a subject. For instance, one or more ASOs may be used in combination.

As used herein, the terms “prevent”, “preventing” and “prevention” refer to the inhibition of the development or onset of a disease or symptoms thereof. In one embodiment, it relates to the administration of the compound to a patient who is known to have an increased risk of developing a certain condition, disorder, or disease.

As used herein, the terms “treat”, “treatment”, and “treating” refer to the halting, ceasing the progression of, or (partially) reversing particular symptoms of a disease or disorder. Beneficial or desired clinical results include, but are not limited to, alleviation of symptoms; diminishment of the extent of a condition, disorder, or disease stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

The terms “subject” or “patient” are used interchangeable and relate to an animal (e.g., mammals) that may need administration of the compound disclosed herein in the field of human or veterinary medicine. In specific embodiments, the subject is a human. The subject may be administered the oligonucleotide disclosed herein for beneficial editing. The subject may be administered the oligonucleotide disclosed herein for compensatory editing.

As used herein, the term “pharmaceutically acceptable” means approved by a regulatory agency. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatine, malt, rice, flour, chalk, silica gel, sodium stearate, glycerol monostearate, talc, sodium chloride, dried skim milk, glycerol, propylene, glycol, water, ethanol and the like. The formulation should suit the mode of administration.

Unless otherwise defined, all technical and scientific terms used herein have the same meaning as commonly understood by one of ordinary skill in the art to which the inventions described herein belong. Methods and materials are described herein for use in the present disclosure; other, suitable methods and materials known in the art can also be used.

### Oligonucleotides

Described herein are, inter alia, chemically modified antisense oligonucleotides (ASOs). While not intending to be bound by any particular theory of operation, it is believed that nucleobase and backbone linkage modifications are useful in stabilising ASOs, improving their editing efficacy, reducing their off-target editing, and/or hydrophobicity. Since the one or more modifications can be synthetically transferred to various oligonucleotide sequences, such modifications have the potential to improve the editing efficacy of oligonucleotides with different target specificities. The ASOs can be used for several purposes associated with “A-to-” conversions. That is, the ASOs are not just limited to correcting G-to-A mutations but are also useful in changing a wildtype sequence into a mutated sequence in order to modulate protein expression and/or function (“beneficial editing”). Thus, the oligonucleotides may be used as active agents to prevent or treat disorders or diseases associated with one or more G-to-A mutations or to change wildtype sequences.

Provided herein is a chemical oligonucleotide for use in site-directed A-to-I editing of a target RNA inside a cell with endogenous adenosine deaminase acting on RNA (ADAR), the oligonucleotide comprising a sequence capable of binding to a target sequence in a target RNA and a central base triplet (CBT) of 3 nucleotides (5′-N₊₁ N₀ N₋₁-3′), wherein N₀ is the central nucleotide directly opposite to a target adenosine in the target RNA that is to be edited, and wherein the oligonucleotide comprises an asymmetry of 25-1-8 in a 5′ to 3′ direction; and comprises: (i) one or more phosphorothioate (PS) linkages at one or more of position +22, position +21, position +11, and position +4; and/or (ii) a 2′-O-methyl(2′-O-Me) modified nucleosides at position +13 and/or position +9.

In some aspects, the present disclosure demonstrates that oligonucleotides containing specific modifications referred to above in the context of an asymmetry of 25-1-8 have beneficial editing properties.

The oligonucleotides disclosed herein can comprise different internucleoside linkages. In a preferred embodiment, while the oligonucleotides disclosed herein comprise different types of internucleoside linkages, the inventors have shown oligonucleotides comprising at least one linkage that is a sulfonylphosphoramidate internucleoside linkage, preferably a methanesulfonyl (mesyl) linkage, have enhanced RNA editing. That is, the inventors have realised that the oligonucleotides disclosed herein do not require all of the internucleoside linkages to carry the same internucleoside linkage (or modification), provided that preferably a minimum level of internucleoside modification is incorporated. Accordingly, in a preferred embodiment, oligonucleotides disclosed herein preferably comprise at least one methanesulfonyl (mesyl) linkage. In one embodiment, oligonucleotides disclosed herein comprise at least one ethanesulfonyl (esyl) linkage. In one embodiment, oligonucleotides disclosed herein comprise at least one propane-1-sulfonyl (prosyl) linkage. In one embodiment, oligonucleotides disclosed herein comprise at least one butane-1-sulfonyl (busyl) linkage. In one embodiment, oligonucleotides disclosed herein comprise at least one pentyl linkage. In one embodiment, oligonucleotides disclosed herein comprise at least one hexane-1-sulfonyl (hesyl) linkage. In one embodiment, oligonucleotides disclosed herein comprise at least one pentyl linkage. In one embodiment, oligonucleotides comprise at least one toluenesulfonyl (tosyl) linkage.

The inventors have also realised that to provide shorter oligonucleotides for RNA editing, and to achieve a beneficial balance of high editing efficacy and low hydrophobicity, it is desirable to incorporate certain backbone linkage and nucleobase modifications and/or mixtures thereof into the oligonucleotides. In particular, depending on the length of the ASO, it is desirable that the ASOs have a mixture of different modifications at the 2′-position of the sugar residue. In a preferred embodiment, introducing mesyl modifications into the core oligonucleotide backbone reduces overall hydrophobicity of the ASO as well as immune activation. Hence, according to one embodiment disclosed herein, the oligonucleotide comprises at least one internucleoside linkage that is a mesyl, esyl, prosyl, pentyl, hesyl, or tosyl linkage. In a preferred embodiment, oligonucleotides disclosed herein comprise at least one internucleoside linkage that is a mesyl linkage.

The specificity sequence of the ASOs disclosed herein may be described as a 5′ to 3′ (antisense) oligonucleotide or polynucleotide sequence. The specificity sequence and target region will be described with reference to the target “A” (adenosine to be edited). The target A is located at the “zero position” within the target sequence. The specificity sequence site within the ASO that is directly opposite the target “A” to be edited is referred to as the zero position (N₀). The downstream positions (i.e., 3′ to the N₀ position) are marked −1, −2, −3, etc. (N₋₁, N₂, N₃, etc.), while the upstream (i.e., 5′ to the N₀ position) positions are numbered +1, +2, +3 (N₊₁, N₊₂, N₊₃, etc.). Accordingly, an oligonucleotide disclosed herein may have a general sequence of 5′- . . . . N₊₅ N₊₄ N₊₃ N₊₂ N₊₁ N₀ N₋₁ N₋₂ N₋₃ N₋₄ N₋₅-3′.

In a separate aspect, provided herein is a chemically modified oligonucleotide for use in site-directed A-to-I editing of a target RNA inside a cell with endogenous adenosine deaminase acting on RNA (ADAR), the oligonucleotide comprising a sequence capable of binding to a target sequence in a target RNA and a central base triplet (CBT) of 3 nucleotides (5′-N₊₁ N₀ N₋₁-3′), wherein N₀ is the central nucleotide directly opposite to a target adenosine in the target RNA that is to be edited, and wherein the oligonucleotide comprises the following core sequence: 5′ . . . . N₊₅ N₊₄ N₊₃ N₊₂ N₊₁ N₀ N₋₁ N₋₂ N₋₃ N₋₄ N₋₅-3′, wherein the oligonucleotide comprises: (i) a phosphorothioate (PS) linkage at position +21 and position +4; (ii) a phosphodiester (PO) linkage at position +23 and position −5, and/or (iii) a sulfonylphosphoramidate internucleoside linkage at one or more positions selected from positions +24, +13, −2 and −8, wherein the sulfonylphosphoramidate internucleoside linkage is of formula (I):

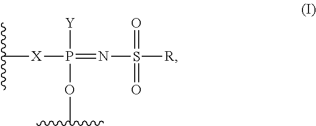

wherein X=Oxygen (O), providing a linkage to a sugar residue; R=a C₁-C₆ alkyl group; and Y=O⁻ or —OH group.

In one embodiment, the sulfonylphosphoramidate internucleoside linkage is a methanesulfonyl (mesyl) linkage. In one embodiment, the sulfonylphosphoramidate internucleoside linkage is an ethanesulfonyl (esyl) linkage. In one embodiment, the sulfonylphosphoramidate internucleoside linkage is a propane-1-sulfonyl (prosyl) linkage. In one embodiment, the sulfonylphosphoramidate internucleoside linkage is a butane-1-sulfonyl (busyl) linkage. In one embodiment, the sulfonylphosphoramidate internucleoside linkage is a hexane-1-sulfonyl (hesyl) linkage.

In one embodiment, the sulfonylphosphoramidate internucleoside linkage is selected from the group consisting of: (i) methanesulfonyl (mesyl) linkage; (ii) ethanesulfonyl (esyl) linkage; (iii) propane-1-sulfonyl (prosyl) linkage; (iv) butane-1-sulfonyl (busyl) linkage; and (v) hexane-1-sulfonyl (hesyl) linkage. In one preferred embodiment, the sulfonylphosphoramidate internucleoside linkage is a mesyl linkage. In one embodiment, the sulfonylphosphoramidate internucleoside linkage is selected from the group consisting of: (i) methanesulfonyl (mesyl) linkage; (ii) ethanesulfonyl (esyl) linkage; (iii) propane-1-sulfonyl (prosyl) linkage; (iv) butane-1-sulfonyl (busyl) linkage; and (v) hexane-1-sulfonyl (hesyl) linkage, preferably wherein the sulfonylphosphoramidate internucleoside linkage is a mesyl linkage.

The oligonucleotides disclosed herein benefit from having a base level of internucleoside linkage modifications. In a preferred embodiment, there will be a methanesulfonyl (mesyl) linkage at one or more of positions +24, +13, −2 and −8. This will have a positive effect on, inter alia, the pharmacokinetics as well as stability, protein binding, intracellular localization, hydrophobicity and cytotoxicity of the ASOs. The oligonucleotides disclosed herein may in addition to the mesyl linkage(s) comprise further internucleoside linkage modifications such as phosphorothioate (PS) linkages. In one embodiment, oligonucleotides disclosed herein have a combination of mesyl and PS linkages and additional internucleoside linkage modifications such as, e.g., esyl, prosyl, busyl, and/or hesyl linkages.

In a preferred embodiment, the chemically modified oligonucleotides disclosed herein comprise at least 4 linkages that are a methanesulfonyl (mesyl) linkage. In one embodiment, the oligonucleotides disclosed herein contain 4 mesyl linkages.

In one embodiment, the mesyl linkage content is at least 5%, that is at least 5% of the internucleoside linkages are methanesulfonyl (mesyl) linkages. In one embodiment, the mesyl linkage content is at least 5%, 12%, 15%, 18%, 21%, 24%, 27%, or 30%. In one embodiment, the mesyl linkage content is between 5%-25%, 12-25%, 12-35%, 12-33%, 12-30%, 12-25%, 12-23%, 12-20%, 12-16% of the internucleoside linkages of the oligonucleotide. In one embodiment, no more than 75%, 33%, 27%, 25%, 22%, 20%, 18%, 15%, or 13% of the linkages are mesyl linkages.

In one embodiment, there are between 4 and 11 internucleoside linkages that are methanesulfonyl (mesyl), ethanesulfonyl (esyl) linkages, propane-1-sulfonyl (prosyl) linkages, butane-1-sulfonyl (busyl) linkages, pentane-1-sulfonyl (pentyl) linkages, or hexane-1-sulfonyl (hesyl) linkages. In one preferred embodiment, there are between 4 and 11 internucleoside linkages that are methanesulfonyl (mesyl) linkages. In one embodiment, between 4 and 10, between 4 and 9, between 4 and 8, or between 4 and 7 internucleoside linkages are methanesulfonyl (mesyl), ethanesulfonyl (esyl) linkages, propane-1-sulfonyl (prosyl) linkages, butane-1-sulfonyl (busyl) linkages, pentane-1-sulfonyl (pentyl) linkages, or hexane-1-sulfonyl (hesyl) linkages. In one embodiment, 5% to 30% of the internucleoside linkages are methanesulfonyl (mesyl), ethanesulfonyl (esyl) linkages, propane-1-sulfonyl (prosyl) linkages, butane-1-sulfonyl (busyl) linkages, pentane-1-sulfonyl (pentyl) linkages, or hexane-1-sulfonyl (hesyl) linkages; or at least 4 internucleoside linkages are methanesulfonyl (mesyl), ethanesulfonyl (esyl) linkages, propane-1-sulfonyl (prosyl) linkages, butane-1-sulfonyl (busyl) linkages, pentane-1-sulfonyl (pentyl) linkages, or hexane-1-sulfonyl (hesyl) linkages, preferably wherein 4 to 8 internucleoside linkages are mesyl linkages. In a preferred embodiment, the internucleoside linkage is a mesyl linkage.

In one embodiment, oligonucleotides disclosed herein comprise no more than 8, 7, 6, 5, or 4 sulfonylphosphoramidate internucleoside linkages. In one embodiment, the oligonucleotide comprises no more than 8, 7, 6, 5, or 4 mesyl linkages. In one embodiment, at least 12% of the internucleoside linkages are mesyl linkages. In one embodiment, between 4 and 8 linkages are mesyl linkages. In one embodiment, oligonucleotides disclosed herein contain 4 mesyl linkages. In one embodiment, oligonucleotides disclosed herein contain 5 mesyl linkages. In one embodiment, oligonucleotides disclosed herein contain 6 mesyl linkages. In one embodiment, oligonucleotides disclosed herein contain 8 mesyl linkages. In one embodiment, oligonucleotides disclosed herein comprise 4 mesyl linkages, wherein said mesyl linkages are located at position +24, position +13, position −2, and position −8. In one embodiment, oligonucleotide disclosed herein comprise as a mesyl linkage at position −6.

In one embodiment, the oligonucleotide comprises no more than 8, 7, 6, 5, or 4 esyl linkages. In one embodiment, at least 12% of the internucleoside linkages are esyl linkages. In one embodiment, between 4 and 8 linkages are esyl linkages. In one embodiment, oligonucleotides disclosed herein contain 4 esyl linkages. In one embodiment, oligonucleotides disclosed herein contain 5 esyl linkages. In one embodiment, oligonucleotides disclosed herein contain 6 esyl linkages. In one embodiment, oligonucleotides disclosed herein contain 8 esyl linkages. In one embodiment, oligonucleotides disclosed herein comprise 4 esyl linkages, wherein said esyl linkages are located at position +24, position +13, position −2, and position −8. In one embodiment, oligonucleotide disclosed herein comprise as a mesyl linkage at position −6.

In one embodiment, the oligonucleotide comprises no more than 8, 7, 6, 5, or 4 prosyl linkages. In one embodiment, at least 12% of the internucleoside linkages are prosyl linkages. In one embodiment, between 4 and 8 linkages are prosyl linkages. In one embodiment, oligonucleotides disclosed herein contain 4 prosyl linkages. In one embodiment, oligonucleotides disclosed herein contain 5 prosyl linkages. In one embodiment, oligonucleotides disclosed herein contain 6 prosyl linkages. In one embodiment, oligonucleotides disclosed herein contain 8 prosyl linkages. In one embodiment, oligonucleotides disclosed herein comprise 4 prosyl linkages, wherein said prosyl linkages are located at position +24, position +13, position −2, and position −8. In one embodiment, oligonucleotide disclosed herein comprise as a prosyl linkage at position −6.

In one embodiment, the oligonucleotide comprises no more than 8, 7, 6, 5, or 4 busyl linkages. In one embodiment, at least 12% of the internucleoside linkages are busyl linkages. In one embodiment, between 4 and 8 linkages are busyl linkages. In one embodiment, oligonucleotides disclosed herein contain 4 busyl linkages. In one embodiment, oligonucleotides disclosed herein contain 5 busyl linkages. In one embodiment, oligonucleotides disclosed herein contain 6 busyl linkages. In one embodiment, oligonucleotides disclosed herein contain 8 busyl linkages. In one embodiment, oligonucleotides disclosed herein comprise 4 busyl linkages, wherein said busyl linkages are located at position +24, position +13, position −2, and position −8. In one embodiment, oligonucleotide disclosed herein comprise as a busyl linkage at position −6.

In one embodiment, the oligonucleotide comprises no more than 8, 7, 6, 5, or 4 pentyl linkages. In one embodiment, at least 12% of the internucleoside linkages are pentyl linkages. In one embodiment, between 4 and 8 linkages are pentyl linkages. In one embodiment, oligonucleotides disclosed herein contain 4 pentyl linkages. In one embodiment, oligonucleotides disclosed herein contain 5 pentyl linkages. In one embodiment, oligonucleotides disclosed herein contain 6 pentyl linkages. In one embodiment, oligonucleotides disclosed herein contain 8 pentyl linkages. In one embodiment, oligonucleotides disclosed herein comprise 4 pentyl linkages, wherein said pentyl linkages are located at position +24, position +13, position −2, and position −8. In one embodiment, oligonucleotides disclosed herein comprise as a pentyl linkage at position −6.

In one embodiment, the oligonucleotide comprises no more than 8, 7, 6, 5, or 4 hesyl linkages. In one embodiment, at least 12% of the internucleoside linkages are hesyl linkages. In one embodiment, between 4 and 8 linkages are hesyl linkages. In one embodiment, oligonucleotides disclosed herein contain 4 hesyl linkages. In one embodiment, oligonucleotides disclosed herein contain 5 hesyl linkages. In one embodiment, oligonucleotides disclosed herein contain 6 hesyl linkages. In one embodiment, oligonucleotides disclosed herein contain 8 hesyl linkages. In one embodiment, oligonucleotides disclosed herein comprise 4 hesyl linkages, wherein said hesyl linkages are located at position +24, position +13, position −2, and position −8. In one embodiment, oligonucleotides disclosed herein comprise as a hesyl linkage at position −6.

In one embodiment, the oligonucleotide comprises no more than 8, 7, 6, 5, or 4 tosyl linkages. In one embodiment, between 4 and 8 linkages are tosyl linkages. In one embodiment, oligonucleotides disclosed herein contain 4 tosyl linkages. In one embodiment, oligonucleotides disclosed herein contain 5 tosyl linkages. In one embodiment, oligonucleotides disclosed herein contain 6 tosyl linkages. In one embodiment, oligonucleotides disclosed herein contain 8 tosyl linkages.

In one embodiment, oligonucleotides disclosed herein comprise 4 tosyl linkages, wherein said tosyl linkages are located at position +24, position +13, position −2, and position −8. In one embodiment, oligonucleotides disclosed herein comprise a tosyl linkage at position +24, position −2, and position −8. In one embodiment, oligonucleotides disclosed herein comprise a tosyl linkage at position +24 and position −8. In one embodiment, oligonucleotides disclosed herein comprise a tosyl linkage at position +24. In one embodiment, oligonucleotides disclosed herein comprise a tosyl linkage at position −8. In one embodiment, oligonucleotides disclosed herein comprise as a tosyl linkage at position −6.

Mesyl linkages are preferably located at position +24, position +13, position −2, and position −8. Further mesyl linkages may be located at any nucleotide position within the oligonucleotides disclosed herein. For instance, one or more further mesyl linkages may be located at internal positions anywhere along the entire length of the oligonucleotide or (only) at the 5′ and/or 3′ terminal ends of the oligonucleotide. Alternatively, in one embodiment, the mesyl linkage is located within a 5′ and/or a 3′ terminus flanking region(s) outside of the CBT (5′-N₊₁ N₀ N₋₁-3′), i.e., upstream of N₊₁ and/or downstream of N₋₁. In one embodiment, the mesyl linkage is located within the CBT, i.e., between position +1 and 0 and/or between positions 0 and −1. In one embodiment, the mesyl linkage is directly (i.e., adjacent to) upstream of N₊₁ (at position +2). In one embodiment, the mesyl linkage is directly downstream (i.e., adjacent to) of N₋₁ (at position −2). In one embodiment, the oligonucleotide comprises a mesyl linkage within the flanking region 3′ to N₀. In one embodiment, the oligonucleotide comprises a mesyl linkage within the flanking region 5′ to N₀. In one embodiment, the oligonucleotide comprises a mesyl linkage within each of the 5′ and 3′ flanking regions. In one embodiment, the oligonucleotide comprises 1, 2, 3, 4, 5, 6, 7, or 8 mesyl linkages within the flanking regions 3′ and/or 5′ to N₀. In one embodiment, the oligonucleotide comprises between 1-20 mesyl linkages 5′ to N₀. In one embodiment, the oligonucleotide comprises between 1-10 mesyl linkages 3′ to N₀.

In one embodiment, a sulfonylphosphoramidate internucleoside linkage (e.g., mesyl, esyl, prosyl, busyl, or tosyl linkage), preferably a mesyl linkage, is located within the 3′ and/or 5′ flanking region(s) outside of the CBT. Since oligonucleotides may vary in overall length, the length of the 3′ and/or 5′ terminal flanking regions of each oligonucleotide may vary in length accordingly. In one embodiment, the flanking regions at the 5′ and 3′ termini have the same length. In one embodiment, the flanking regions at the 5′ and 3′ termini have different lengths.

In one embodiment, the oligonucleotide comprises 2, 3, 4, 5, 6 or 7 sulfonylphosphoramidate internucleoside linkages within a 3′ and/or 5′ flanking region(s) outside of the CBT. In one preferred embodiment, the oligonucleotide comprises 2, 3, 4, 5, 6 or 7 mesyl modifications within a 3′ and/or 5′ flanking region(s) outside of the CBT. In one embodiment, the oligonucleotide comprises at least 2, 3, 4, 5, 6, or 7 mesyl modifications within a 3′ and/or 5′ flanking region(s) outside of the CBT. In one embodiment, the 5′ terminus flanking region comprises the terminal 10, 9, 8, 7, 6, 5, 4, 3, 2 or 1 nucleotide(s) of the oligonucleotide, preferably wherein the 5′ terminus flanking region comprises the outermost 6, 5, 4, 3, 2, or 1 nucleotide(s). In one embodiment, the 3′ terminus flanking region comprises the terminal 7, 6, 5, 4, 3, 2 or 1 nucleotide(s) of the oligonucleotide, preferably wherein the 3′ terminus flanking region comprises the outermost 4, 3, 2, or 1 nucleotide(s).

In one embodiment, the oligonucleotide comprises 1, 2, or 3 mesyl linkages within the 3′ and/or 5′ terminus flanking region(s). In one embodiment, the 1, 2, or 3 mesyl linkages within the 3′ and/or 5′ terminus flanking region(s) are between the terminal 1, 2, 3 and 4 nucleotides of the 5′ and/or 3′ terminus. That is, in one embodiment, the 1, 2, or 3 mesyl linkages are located between the outermost 5 nucleotides of the 5′ and/or outermost 4 nucleotides of the 3′ terminus of the oligonucleotide. In one embodiment, the 1 or 2 mesyl linkages are located between the outermost 2 or 3 nucleotides of the 5′ and/or outermost 2 or 3 nucleotides of the 3′ terminus of the oligonucleotide.

In other embodiments, the oligonucleotide comprises a mesyl linkage between the terminal and penultimate nucleotide of the 5′ terminus and a mesyl linkage between the terminal and penultimate nucleotide of the 3′ terminus. In other embodiments, the oligonucleotide comprises 2 mesyl linkages at the 5′ terminus, which are placed between the terminal 3 nucleotides of the 5′ terminus. In other embodiments, the oligonucleotide comprises 2 mesyl linkages at the 3′ terminus, which are placed between the terminal 3 nucleotides of the 3′ terminus. In one embodiment, the oligonucleotide comprises 2 mesyl linkages at the 5′ terminus, which are placed between the terminal 3 nucleotides of the 5′ terminus and 2 mesyl linkages at the 3′ terminus, which are placed between the terminal 3 nucleotides of the 3′ terminus.

In one embodiment, a mesyl linkage is located between any two of the nucleotide positions of the oligonucleotide. That is, in an oligonucleotide with a length of 34 nt, a mesyl linkage may be located at any of the 34 positions of the oligonucleotide (e.g., at position 5′-+24, +23, +22, [ . . . ], . . . 0, . . . [ . . . ], −3, −4, −5, −6, −7, −8-3′). In one embodiment, a mesyl linkage is located between the outermost 1-5, 1-6, 1-7, 1-8, 1-9 or 1-10 nucleotides. In one embodiment, the mesyl linkage is located at one or more of positions+positions +28, +27, +26, +25, +24, +23, +22, +21, +20, +19, +14, +13, +12, +11, +10, +5, +4, +3, −2, −5, −7, −8, −9, −11, −12, −13, −14, −15, −16, −17, −18, and/or −19. In one embodiment, the mesyl linkage is positioned at one or more of the following positions +27, +26, +25, +24, +23, +22, +21, +20, +19, +13, +12, +11, +6, +5, +4, 2, −6, −7, and −8. In one embodiment, the mesyl linkage is located at one or more of the following positions selected from: +24, +23, +21, +13, +4, −2, −6, −7, and −8. In one embodiment, the mesyl linkage is located at positions +24, +13, −2, and −8. In one embodiment, the mesyl linkage is located at positions +24, +13, −2, −6 and −8.

In some embodiments, there is no mesyl linkage at one or more of the following positions: +21, +19, +18, +17, +16, +15, +14, +10, +9, +8, +7, +6, +3, +2, +1, 0, −1, −2, −3, −4, −5, −7, −9, and −10. In one embodiment, there is no mesyl linkage at position +4. In one embodiment, there is no mesyl linkage at position +21. In one embodiment, there is no mesyl linkage at position +4 and/or +21.

In one embodiment, the mesyl linkage is located at position +24. In one embodiment, the mesyl linkage is located at position +23. In one embodiment, the mesyl linkage is located at position +21. In one embodiment, the mesyl linkage is located at position +19. In one embodiment, the mesyl linkage is located at position +18. In one embodiment, the mesyl linkage is located at position +13. In one preferred embodiment, the mesyl linkage is located at position −2. In one embodiment, the mesyl linkage is located at position −5. In one embodiment, the mesyl linkage is located at position −6. In one embodiment, the mesyl linkage is located at position −7. In one embodiment, the mesyl linkage is located at position −8.

Accordingly, in one embodiment, a mesyl linkage is located in the 5′ and/or 3′ flanking regions of the ASO. In one embodiment, a mesyl linkage is located at position +24 and/or at position −8. In one embodiment, the oligonucleotide comprises mesyl linkages at positions +24, +13, −2, and −8. In one embodiment, the oligonucleotide comprises mesyl linkages at positions +24, +13, −2, −6 and −8. In one embodiment, the oligonucleotide comprises mesyl linkages at positions +24, −2, and −8. In one embodiment, the oligonucleotide comprises mesyl linkages at positions +24 and −8.

Alternatively, oligonucleotides disclosed herein may comprise internucleoside linkage modifications at those positions described for mesyl. For instance, the oligonucleotides disclosed herein may comprise esyl, prosyl, busyl, and/or hesyl linkages at the terminal nucleotides of the ASO disclosed herein, i.e., between the terminal and penultimate nucleotide of the 5′ and/or 3′ end of the ASO. In one embodiment, an esyl, prosyl, busyl, and/or hesyl linkage is located in the 5′ and/or 3′ flanking regions of the ASO. In one embodiment, an esyl linkage is located at position +24 and/or at position −8. In one embodiment, a prosyl linkage is located at position +24 and/or at position −8. In one embodiment, a busyl linkage is located at position +24 and/or at position −8. In one embodiment, a hesyl linkage is located at position +24 and/or at position −8.

The chemically modified oligonucleotides disclosed herein may be symmetrical, which means that the two nucleotide sequences adjacent to the CBT have the same length, or not symmetrical (asymmetrical or asymmetric design), which means that the two sequences flanking the CBT, i.e., the regions 5′ and 3′ to the CBT and/or position N₀, have different lengths. The asymmetric design enables a more flexible use of the sequence space around the target. Hence, in one embodiment, the oligonucleotide comprises an asymmetric design. In one embodiment, the oligonucleotide has: (i) a length of 20 to 29 nt located 5′ to N₀, and (ii) a length of 5 to 20 nt located 3′ to N₀. In one embodiment, the oligonucleotide has: (i) a length of 23 to 27 nt located 5′ to N₀, and (ii) a length of 6 to 10 nt located 3′ to N₀.

The oligonucleotides disclosed herein may be of any length suitable to achieve an edit. The oligonucleotides disclosed herein are preferably at least 30, more preferably at least 31 nucleotides (nt) long. In some embodiments, the oligonucleotides range from 31-80 nt, 34-80 nt or 31-60 nt in length. In one embodiment, the oligonucleotide has a length of 31-40 nt. In some embodiments, the oligonucleotide has a length of 31-38 nt. In some embodiments, the oligonucleotide has a length of 31-34 nt. In some embodiments, the oligonucleotide has a length of 32-34 nt. In one embodiment, the oligonucleotide has a length of 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nt. In one embodiment, the oligonucleotide has a length of 32 nt. In one embodiment, the oligonucleotide has a length of 33 nt. In one embodiment, the oligonucleotide has a length of 34 nt. In one embodiment, the oligonucleotide has a length of 35 nt. In one embodiment, the oligonucleotide has a length of 36 nt. In one embodiment, the oligonucleotide has a length of 37 nt. In one embodiment, the oligonucleotide has a length of 38 nt. In certain embodiments, the oligonucleotide has a length of 32 or 34 nt. In one embodiment, the oligonucleotide has a length of no more than 34 nt. In one embodiment, the oligonucleotide has a length of no more than 38, 39, 40, 41, 42, 43, 44, or 45 nt. Ranges and lengths intermediate to the above recited ranges and lengths are also contemplated to be part disclosed herein.

Without being bound by any theory, inventors submit that the ideal asymmetry for each target might depend on the length and the specific underlying sequence of the particular oligonucleotide. The inventors previously showed that for asymmetric ASOs, a shorter overall oligoribonucleotide is sufficient for high editing efficacy compared to the symmetric design (WO 2022/253810). It is known that ADAR works as an asymmetric dimer with a footprint of up to 50 bp. While some substrates are more efficiently edited by the deaminase domain alone rather than by the full-length protein, the opposite holds true for other substrates. This suggests that depending on the size of the target/drug RNA helix, ADAR might bind in different ways. This leads to a situation, wherein, depending on the length of the ASO, specific (a) symmetries on the target adenosine and specific modifications patterns (e.g., sugar and internucleoside linkage modifications) are preferred. For an optimal binding of the deaminase, a short 3′ terminus seems to be sufficient (at least 5 nt beside the CBT). On the other hand, the 5′ terminus may provide binding space for the dsRBDs and thus typically requires more nucleotides (at least 19 nt beside the CBT).

The oligonucleotides disclosed herein have the following structural scheme: (length of 5′ terminus)-(1)-(length of 3′ terminus), wherein 1 corresponds to the central nucleotide of the CBT opposite of the target A (N₀). For example, an ASO disclosed herein with a length of 38 nt and an asymmetry of “29-1-8”, has a 5′ terminus that is 29 nt long and a 3′ terminus that is 8 nt long. In one embodiment, the chemically modified oligonucleotide disclosed herein comprises an asymmetric design, wherein there is a different number of nucleotides 5′ and 3′ of N₀. For instance, there may be 20-30 nt at the 5′ terminus (5′ to N₀) and 5-20 nt at the 3′ terminus (3′ to N₀). In some embodiments, the 5′ terminus is shortened to a length of 25 nt 3′ of N₀. In some embodiments, the 3′ terminus is shortened to a length of 8 nt 3′ of N₀. In one embodiment, there are 25 nt 5′ to N₀, and 8 nt 3′ to N₀ (25-1-8 asymmetry). In one embodiment, there are 23 nt 5′ of N₀ and 8 nt 3′ of N₀ (23-1-8 asymmetry). In one embodiment, the oligonucleotide has a length of 34 nt and an asymmetry of 25-1-8 in a 5′ to 3′ direction. This is because the specific chemical modification pattern of the oligonucleotide in the context of an asymmetry of 25-1-8 results in beneficial editing properties.

In one preferred embodiment, the oligonucleotide has an asymmetry of 25-1-8 in a 5′ to 3′ direction, and a mesyl linkage is located at positions +24, +13, −2, and −8. In one preferred embodiment, a further mesyl linkage is located at position +21, −7 and/or −23.

The inventors have further realised that the length of the oligonucleotide can be shortened without losing its editing efficacy provided the oligonucleotide comprises additional 2′-sugar and internucleoside linkage modifications. In one embodiment, the oligonucleotide has an asymmetry of 25-1-8 and comprises between 10 and 15 2′-F modifications. In one embodiment, the oligonucleotide has an asymmetry of 25-1-8 and comprises 10 2′-F modifications. In one embodiment, the oligonucleotide has an asymmetry of 25-1-8 and comprises 11 2′-F modifications.

Furthermore, the ASO disclosed herein may comprise 2′-fluoro (2′-F) and/or 2′Ome modifications. In one embodiment, at least 20%, 25%, 30%, 35%, 40%, 45%, or 50% nucleotides are fluoro (F)-modified at the 2′ position of the sugar residue. In one embodiment, the oligonucleotide comprises 10 to 15 2′-F modifications. In one embodiment, the oligonucleotide comprises 10 2′-F modifications. In one embodiment, the oligonucleotide comprises 11 2′-F modifications. In one embodiment, a 2′-F modification is located at one or more of the following positions selected from the group consisting of: +22, +21, +19, +16, +15, +13, +11, +7, +5, +2, −3 and −5. In one embodiment, the 2′-F modification is located at position +22, +21, +19, +16, +15, +13, +11, +7, +5, +2, and −3. In one embodiment, the 2′-F modification is located at position +22, +21, +19, +16, +15, +11, +7, +5, +2, and −3. In one embodiment, the 2′-F modification is located at position +22, +21, +19, +16, +15, +11, +7, +5, +2, −3, and −5.

In one embodiment, at least 25%, preferably 30-70%, more preferably 35-50% of the chemical modifications are 2′-O-methyl(2′-OMe) modifications. In one embodiment, the oligonucleotide comprises 12 2′-OMe modifications. In one embodiment, the oligonucleotide comprises 13 2′-OMe modifications. In one embodiment, the oligonucleotide comprises 14 2′-OMe modifications. In one embodiment, the oligonucleotide comprises 15 2′-OMe modifications. In one embodiment, the oligonucleotide comprises 12 2′-OMe modifications at positions +25, +24, +20, +18, +10, +9, +4, −2, −5, −6, −7, and −8. In one embodiment, the oligonucleotide comprises 13 2′-OMe modifications at positions +25, +24, +23, +20, +18, +12, +10, +4, −2, −5, −6, −7, and −8. In one embodiment, the oligonucleotide comprises 13 2′-OMe modifications at positions +25, +24, +23, +20, +18, +12, +10, +9, +4, −2, −6, −7, and −8. In one embodiment, the oligonucleotide comprises 14 2′-OMe modifications at positions +25, +24, +23, +20, +18, +13, +12, +10, +9, +4, −2, −6, −7, and −8. In one embodiment, the oligonucleotide comprises 15 2′-OMe modifications at positions +25, +24, +23, +20, +18, +13, +12, +10, +9, +4, −2, −5, −6, −7, and −8.

In one embodiment, each RNA nucleoside of the oligonucleotide is replaced by either a 2′-modified RNA or DNA. In addition to the methanesulfonyl (mesyl) linkages the oligonucleotide may comprise a phosphodiester (PO) linkage and/or internucleoside linkage modifications such as phosphorothioate (PS). In one embodiment, the oligonucleotide comprises one or more internucleoside linkages selected from the group consisting of PO and PS. In one embodiment, the further internucleoside linkage is a PS linkage. In one embodiment, the internucleoside linkage modification is a 3′-3′ or 5′-5′ phosphate ester bonds (3′-P-3′ and 5′-P-5′). In one embodiment, the natural 3′-5′ phosphodiester linkage is replaced by modified internucleoside linkages. In some embodiments, the naturally occurring one or more PO linkages are replaced by modified internucleoside linkages to introduce one or more PS linkages or non-phosphorus derived internucleoside linkages. In one embodiment, an internucleoside linkage is a PS linkage. In one embodiment, an internucleoside linkage is a stereorandom PS linkage. In one embodiment, an internucleoside linkage is a chirally controlled PS linkage. In one embodiment, an internucleoside linkage is not a chirally controlled PS linkage.

In one embodiment, at least 40% of linkages are PS linkages. In one embodiment, between 50% and 65% of linkages are PS linkages.

In one embodiment, the oligonucleotide comprises 9-21 PS linkages. In one embodiment, the oligonucleotide comprises 18 PS linkages. In one embodiment, the oligonucleotide comprises 19 PS linkages. In one embodiment, the oligonucleotide comprises 20 PS linkages. In one embodiment, less than 50%, less than 45%, less than 40%, or less than 35% of the internucleoside linkages are PO linkages. In one embodiment, less than 35% of the internucleoside linkages are PO linkages. In one embodiment, 50%-70% of the internucleoside linkages are PO linkages. In one embodiment, 52%-65% of the internucleoside linkages are PO linkages.

The positioning of additional, chemically distinct internucleoside linkages within the oligonucleotide disclosed herein plays an important role when determining a balance between high editing yields, a long half-life and cytotoxicity. To obtain improved stabilization and editing, oligonucleotide linkages may be modified at particular positions within the oligonucleotide sequence (formula I). The inventors have found that placement of PO linkages at specific positions within the oligonucleotide stabilises the oligonucleotide and contributes to enhanced target editing. Accordingly, in one embodiment, the oligonucleotide comprises a PO linkage at position +23, +22, +17, +16, +12, +11, +9, +8, +7, +5, +3, +2, −3, −5 and/or −6. In one embodiment, the oligonucleotide comprises a PO linkage at position +12. In one embodiment, the oligonucleotide comprises a PO linkage at positions +23, +17, +16, +9, +8, +7, +5, +3, +2, −5, and −6. In one embodiment, the oligonucleotide comprises a PO linkage at positions +23, +22, +17, +16, +11, +8, +7, +5, +3, +2, and −5. In one embodiment, the oligonucleotide comprises a PO linkage at positions +23, +17, +16, +12, +8, +5, +3, +2, −3, and −5. In one embodiment, the oligonucleotide comprises a PO linkage at positions +23, +17, +16, +12, +8, +5, +3, +2, and −5. In one embodiment, the oligonucleotide comprises a PO linkage at positions +23, +17, +16, +12, +8, +5, +3, +2, −3, and −5. In one embodiment, the oligonucleotide has PO linkages only at these positions.

In one preferred embodiment, the oligonucleotide comprises a PS linkage at position +9 and a PO linkage at position +12. In one embodiment, the oligonucleotide further comprises at least one PO, PS, and/or sulfonylphosphoramidate internucleoside linkage selected from the group consisting of esyl, prosyl, busyl and hesyl linkage. In one preferred embodiment, the oligonucleotide comprises a PS linkage at position +9 and a PO linkage at position +12. In one embodiment, the oligonucleotide further comprises at least one PO, PS, and/or mesyl linkage.

In addition to the design features mentioned before, the chemically modified oligonucleotides may comprise at least one nucleotide of the CBT modified at the 2′-position of the sugar base or being deoxyribonucleosides, which permits added stabilization against nuclease digestion. Hence, in certain embodiments, the CBT is chemically modified. The CBT (5′ . . . . N₊₁-N₀-N₋₁- . . . 3′) may carry different modifications and permutations of the various modifications. That is, positions N₊₁, N₀ and/or N₋₁ may carry modifications at the 2′ position. In one embodiment, only one position within the CBT is chemically modified. In one embodiment, two positions within the CBT are chemically modified. In one embodiment, all positions within the CBT are chemically modified.

In one embodiment, at least one of the three oligonucleotides of the CBT is a deoxyribonucleotide. In one embodiment, at least one of the three nucleotides of the CBT is chemically modified at the 2′ position of the sugar residue. In one embodiment, at least one of the three oligonucleotides is 2′-FANA-modified. In one embodiment, at least one of the three oligonucleotides is-O-methyl-modified. In one embodiment, at least one of the three oligonucleotides is 2′-F-modified. In some embodiments, at least one of the three nucleotides of the CBT is chemically modified at the 2′-position of the sugar residue, a deoxyribonucleoside, or a combination thereof. In one embodiment, the chemical modification at the 2′ position is one or more of the following: (i) N₊₁ is 2′-fluoro (2′-F), 2′-fluoroarabinoside (2′-FANA), deoxyribonucleic acid (DNA), 2′-O-Methoxyethyl(2′-MOE) or 2′-O-Methyl(2′-OMe); and/or (ii) N₀ is 2′-FANA or DNA; and/or (iii) N₋₁ is 2′-FANA, DNA or 2′-OMe. In one embodiment, N₋₁ is 2′-OMe. In one embodiment, (i) N₊₁ is 2′-O-Methoxyethyl(2′-MOE); (ii) N₀ is DNA; and (iii) N₋₁ is DNA.

In one embodiment, at least two of the three nucleotides of the CBT are chemically modified at the 2′-position of the sugar residue, a deoxyribonucleoside, or a combination thereof. In some embodiments, N₊₁ is 2′-F, 2′-FANA, DNA, or 2′-OMe; and/or N₀ is 2′-FANA or DNA; and/or N₊₁ is 2′-FANA, DNA, or 2′-O-methyl. In one embodiment, N₊₁ is DNA. In one embodiment, N₊₁ is 2′-F. In one embodiment, N₊₁ is 2′-FANA. In one embodiment, N₀ is 2′-FANA. In one embodiment, N₀ is DNA. In one embodiment, N₋₁ is 2′-FANA. In one embodiment, N₋₁ is DNA.

According to one embodiment, each of the three nucleosides of the CBT is either singularly or a combination of: (a) a deoxyribonucleotide; and/or (b) 2′-fluoroarabinoside (2′-FANA) modification; and/or (c) 2′-O-methyl(2′-OMe) modification; and/or (d) 2′-fluoro (2′-F) modification.

In one embodiment, the middle or centre nucleotide (N₀) of the CBT does not comprise a 2′-sugar modification, although it may be a deoxyribonucleotide. In one embodiment, N₀ does not comprise a 2′-alkyl modification. In one embodiment, N₀ does not comprise a 2′-OMe modification.

In some embodiments, the CBT comprises no cytosine analogues. In one embodiment, the CBT does not comprise pseudoisocytidine (PiC) or 6-amino-5-nitro-2 (1H)-pyridone. In one embodiment, the CBT does not comprise a Benner's base Z (dZ). In other embodiments, the CBT does not comprise a cytidine analogue such as, for example, 5-hydroxyC-H+, 5-aminoC-H+ and 8-oxoA (syn). Hence, in one embodiment, (i) N₀ comprises no 2′-sugar modification, preferably wherein N₀ comprises no 2′-alkyl modification (e.g., no 2′OMe modification), and/or (ii) the CBT comprises no cytosine analogues.

The oligonucleotides disclosed herein may also comprise modifications to the nucleotides positioned outside of the CBT. For example, the sugar or base of the one or more nucleotides may be modified. This is typically to provide greater resistance to nuclease attack in vivo. In one embodiment, the oligonucleotide incorporates modifications at one or more of the 2′-position of the nucleotides and these modifications are composed of different groups. In one embodiment, the oligonucleotide comprises a mixture of 2′-O-alkyl, 2′-F, 2′-MOE, 2′-FANA and/or LNA modifications. The oligonucleotides may comprise any permutation of these 2′-sugar modifications.

In one embodiment, at least 50%, more preferably at least 80% of the nucleotides outside the CBT are modified independently from another at the 2′ position of the sugar residue. In one embodiment, the 2′-sugar modification is selected from 2′-F, 2′-FANA, 2′-O-alkyl, 2′-O-methoxyethyl(2′-MOE), and/or locked nucleic acid (LNA). In one embodiment, the 2′-O-alkyl modification is a 2′-OMe modification. However, the oligonucleotides disclosed herein preferably do not contain blocks of more than 4 continuous nucleotides modified in the same way. In one embodiment, the oligonucleotides preferably do not contain blocks of more than 4 continuous 2′-OMe- or 2′-F-modified nucleotides. In one embodiment, the oligonucleotides preferably do not contain blocks of more than 4 continuous 2′-OMe-modified nucleotides. In one embodiment, the oligonucleotides do not contain blocks of more than 3 continuous 2′-F-modified nucleotides. In one embodiment, the oligonucleotide comprises a 2′-O-Me modified nucleoside at position +9. In one embodiment, the oligonucleotide comprises a 2′-O-Me modified nucleoside at position +13. In one embodiment, the oligonucleotide comprises a 2′-OMe modified nucleoside at position +23, position +12, position +9, and/or position −8.

In one embodiment, a 2′-sugar modification is a 2′-O-alkyl modification. In one embodiment, a 2′-O-alkyl modification is a 2′-OMe, 2′-O-ethyl, or 2′-O-propyl modification. In some embodiment, a 2′-sugar modification is a 2′-MOE modification. In one embodiment, a 2′-sugar modification is 2′-OMe. In some embodiments, a 2′-sugar modification is 2′-MOE. In one embodiment, the oligonucleotide comprises one or more 2′-methoxyethyl(2′-MOE) modified nucleosides. In one embodiment, the oligonucleotide comprises a 2′-MOE modified nucleoside at position +23, position +12, position +9, and/or position −8. In one embodiment, a 2′-MOE modification is located as position +9.

In a preferred embodiment, a mixture of 2′-F- and 2′-O-alkyl-modifications is beneficial to editing and that a minimum of 10% of each is desirable. In some embodiments, the oligonucleotide comprises a mixture of 2′-F- and 2′-O-alkyl-modifications and a minimum of 15% of each 2′-F- and 2′-O-alkyl-modifications. In some embodiments, the oligonucleotide comprises a mixture of 2′-F- and 2′-O-alkyl-modifications and a minimum of 20% of each 2′-F- and 2′-O-alkyl-modifications. In some embodiments, the oligonucleotide comprises a mixture of 2′-F- and 2′-O-alkyl-modifications and a combined minimum of 15%-20%, 20-30%, 30%-40%, 40-50% or 40-60% of 2′-F- and 2′-O-alkyl-modifications. In one embodiment, the oligonucleotide comprises a 2′-fluoro (2′-F) modified nucleoside or a 2′-O-Me modified nucleoside at position −5 and/or position +13. In one embodiment, the oligonucleotide further comprises at least one 2′-O-Me modified nucleoside and/or at least one 2′-F modified nucleoside.

In some embodiments, the oligonucleotide comprises at least 10% of 2′-F, 2′-OMe, 2′-MOE and/or 2′-FANA modifications. In some embodiments, the oligonucleotide comprises at least 15%, 20%, 25%, 30%, 35%, 40% of 2′-F, 2′-OMe, 2′-MOE or 2′-FANA modifications. In some embodiments, the oligonucleotide comprises at least 15%, 20%, 25%, 30%, 35%, 40% of 2′-F, 2′-OMe, 2′-MOE and 2′-FANA modifications.

The oligonucleotides disclosed herein may not carry a 2′-sugar modification in some of the positions. In one embodiment, not all nucleotides comprise a 2′-alkyl modification. In some instances, the 2′-O-alkyl modification is not a 2′-MOE. In some instances, the 2′-modification is not a 2′-OMe, 2′-F or 2′-LNA modification. In some embodiments, not all 2′-sugar modifications are 2′-O-alkyl modifications. In some embodiments, not all 2′-sugar modifications are 2′-F modifications. In some embodiments, not all 2′-sugar modifications are 2′-MOE modifications.

In one embodiment, the oligonucleotide comprises at least one thymine (5-methyluracil) and/or or 5-methylcytosine (5′MeC). In one embodiment, the thymine (5-methyluracil) is located at position +9. In one embodiment, the thymine (5-methyluracil) carries a 2′MOE modification. In one embodiment, the 5′MeC is located at position +23, position +12 and/or position −8. In one embodiment, the 5′MeC carries a 2′MOE modification.

The oligonucleotides disclosed herein may comprise RNA and/or DNA. Also, the oligonucleotides may comprise modifications at the 2′-position of the sugar residue. In one embodiment, 20-100%, 30-100%, 40-100%, 50-100%, 60-100%, 70-100%, 80-100%, or 90-100% of nucleotides are DNA or 2′-modified. In one embodiment, 20-100% of nucleotides are DNA or 2′-modified. In one embodiment, 50-100% of nucleotides are DNA or 2′-modified nucleotides. In one embodiment, 100% of nucleotides are DNA or 2′-modified nucleotides. In one embodiment, 30-95%, 40-95%, 40-90%, 50-95%, 50-90%, 60-95% or 60-90% of nucleotides are DNA or 2′-modified nucleotides. In some embodiments, the DNA content of the oligonucleotide is between 0-10%. In one embodiment, the DNA content is between 1-9%, preferably between 1-7%. In one embodiment, the DNA content is between 1-6%, Preferably between 1-5%. In one embodiment, the DNA content is between 1-4%, optionally between 1-3%. In one embodiment, the DNA content is less than 10%, 9%, 8%, 7%, 6%, 5%, 4%, or 3%.

In one embodiment, no more than 90%, 80%, 70%, 60%, 50%, 40%, 30%, 20% or 10% of nucleotides outside the CBT are deoxynucleotides. In some embodiment, no more 10%, optionally no more than 8%, optionally no more than 6% of nucleotides outside the CBT are deoxynucleotides. In one embodiment the above percentages are satisfied with only 2′-modified nucleotides and no DNA. In some embodiments, the oligonucleotide comprises no DNA. In one embodiment, only 1 nucleotide outside the CBT is deoxynucleotide. In one embodiment, no more than 2 nucleotides outside the CBT are deoxynucleotides. In one embodiment, no more than 4 nucleotides outside the CBT are deoxynucleotides. In one embodiment, no more than 3 nucleotides outside the CBT are deoxynucleotides. In one embodiment, no more than 5 nucleotides outside the CBT are deoxynucleotides. In one embodiment, no more than 6 nucleotides outside the CBT are deoxynucleotides. In some embodiment, no more than 7 nucleotides outside the CBT are deoxynucleotides.

The oligonucleotides disclosed herein may specifically comprise 2′-F and/or 2′-OMe modifications. In one embodiment, the oligonucleotide comprises one or more 2′-F modifications. In one embodiment, no more than 5%, 10%, 20%, 30%, 40%, 50%, 60%, or 70% of nucleotides are 2′-F-modified. In one embodiment, at least 5%, 10%, 20%, 30%, 40%, 50%, or 60% of nucleotides are 2′-F-modified. In one embodiment, no more than 35% of nucleotides are 2′-F modified. In one embodiment, 30-60% of nucleotides are 2′-F-modified. In one embodiment, 20-70%, preferably 30-45%, of nucleotides are 2′-F-modified. In one embodiment, 35-65% of nucleotides are 2′-F-modified.

Oligonucleotides may also comprise 2′-O-methyl(2′-OMe) modifications. In one embodiment, the oligonucleotide comprises one or more 2′-OMe modifications. In one embodiment, no more than 20%, 30%, 40%, 50%, 60%, or 70% of nucleotides are 2′-OMe-modified. In one embodiment, 20-60% of nucleotides are 2′-OMe-modified. In one embodiment, at least 20%, preferably 30-70%, more preferably 40-60% of the chemical modifications outside the CBT are 2′-O-methyl substituents.

The chemically modified oligoribonucleotide according to the disclosure may comprise a general core sequence of formula I: 5′ . . . . N₊₅ N₊₄ N₊₃ N₊₂ N₊₁ N₀ N₋₁ N₋₂ N₋₃ N₋₄ N. 5 . . . 3′ (formula I). In this formula I there is a Central Base Triplet (CBT) of three nucleotides, whereby the central nucleotide is designated by “0”. The nucleotide designated as “0” and the two nucleotides directly adjacent to nucleotide “0” having the number-1 and +1 are designated as a Central Base Triplet, whereby the central nucleotide designated as “0” is directly opposite to the target adenosine in the target RNA. The nucleotide of formula I is flanked at the 5′- and (adjacent to nucleotide +5) and at the 3′-end (adjacent to nucleotide-4) with further oligonucleotide sequences, which may have either the same length or different lengths.

Also, due to cytotoxicity and non-specific protein binding, it is desirable to reduce the overall PS content of the oligonucleotide. The oligonucleotides disclosed herein comprise a PS linkage at positions +21 and +4. In one embodiment, the oligonucleotide comprises a PS linkage at position +22, +20, +19, +18, +15, +14, +12, +11, +10, +9, +7, +6, +1, 0, −1, −3, −4, −6, and/or −7. In one embodiment, the oligonucleotide has PS only at these positions.

Oligonucleotides of different lengths may require a different mixture of particular 2′-modifications and internucleoside linkage modifications in order to provide optimal RNA editing. The shorter the oligonucleotide, the better may be the endosomal escape. Moreover, cytotoxicity of the particular oligonucleotide may also depend on its length. Also, shorter oligonucleotides may experience higher specificity. On the other hand, while longer oligonucleotides may bind stronger or faster to their respective RNA target, editing-boosting bulges, mismatches and wobbles may also work better in long oligonucleotides. As a result, there is a benefit and/or trade-off for both long and short oligonucleotides disclosed herein.

In some embodiments, at least 20%, 30%, 40%, or 50% nucleotides are fluoro (F)-modified at the 2′ position of the sugar residue, optionally wherein the 2′-F modification is at one or more of the following positions: +22, +21, +19, +16, +15, +13, +11, +7, +5, +2, −3, and/or −5. In one embodiment, the 2′-F modification is at positions +22, +21, +19, +16, +15, +11, +7, +5, +2, and −3.

In one embodiment, the oligonucleotide comprises an internucleoside linkage modification selected from the group consisting of PS, 3′-methylenephosphonate, 5′-methylenephosphonate, 3′-phosphoroamidate, 2′-5′-phosphodiester, and PN. In a preferred embodiment, the internucleoside linkage modification is a PS linkage. In one embodiment, the internucleoside linkage modification is a 3′-methylenephosphonate linkage. In one embodiment, the internucleoside linkage modification is a 5′-methylenephosphonate linkage. In one embodiment, the internucleoside linkage modification is a 3′-phosphoroamidate linkage. In one embodiment, the internucleoside linkage modification is a 2′-5′-phosphodiester linkage. In one embodiment, the at least one internucleoside linkage modification is a PS linkage. In one embodiment, the oligonucleotide contains a continuous stretch of PS linkages. In one embodiment, the continuous stretch of PS linkages is 2, 3, 4, or 5 linkages long.

2′-MOE residues are used for splice switching oligonucleotides and typically have very low cytotoxicity. However, due to their bulkiness they are not well accepted in larger quantities. The inventors disclosed herein have realized that 2′-MOE modifications could be placed 5′ and 3′ of the CBT and/or at the termini of the oligonucleotides without reducing the overall editing of the ASO. Specifically, the inventors realised that the amount of 2′-MOE modifications could be limited to about no more than 5 nucleotides to still obtain good RNA editing. Therefore, in one embodiment, the oligonucleotide comprises no more than 5 2′-MOE modifications. In one embodiment, there is a 2′-MOE at position +17. In one embodiment, there is a 2′-MOE at position +14. In one embodiment, there is a 2′-MOE at position +12. In one embodiment, there is a 2′-MOE at position +9. In one embodiment, there is a 2′-MOE at position +6. In one embodiment, there is a 2′-MOE at position +3. In one embodiment, there is a 2′-MOE at position +1. In one embodiment, there is a 2′-MOE at position −4. In one embodiment, there is a 2′-MOE at positions +17, +14, +8, +6, +3, +1, and −4.

In one embodiment, the oligonucleotide does not comprise any PN modifications. In one embodiment, the oligonucleotide does not comprise any PN modifications at the outermost three nucleotides of the 3′ terminus and/or the 5′ terminus.

In one embodiment, the oligonucleotide does not comprise any methylphosphonate (MP) linkages.

Locked nucleic acid (LNA) is a structurally rigid modification that increases the binding affinity of a modified oligonucleotide. In one embodiment, the oligonucleotide comprises terminal LNAs, wherein the oligonucleotide comprises 2 to 5 LNAs at each terminus. In one embodiment, the oligonucleotide comprises 2 LNAs at each terminus. In one embodiment, the oligonucleotide comprises 2 LNAs at the 5′ terminus. In one embodiment, there is no 2′-MOE modification within the CBT. Oligonucleotides may have a general structure of (length of 5′ terminus)-(1)-(length of 3′ terminus), wherein 1 corresponds N₀ or to the central nucleotide of the CBT opposite of the target A. The region 3′ to N₀ is referred to as the “3′ flanking region” or “3′ terminus flanking region”. The region 5′ to N₀ is referred to as the “5′ flanking region” or “5′ terminus flanking region”. In one embodiment, the 3′ terminus flanking region comprises the terminal 6, 5, 4, 3, 2 or 1 nucleotide(s) of the 3′ end of the oligonucleotide; and the 5′ terminus flanking region comprises the terminal 10, 9, 8, 7, 6, 5, 4, 3, 2 or 1 nucleotide(s) of the 5′ end of the oligonucleotide. In one embodiment, the 5′ terminal flanking region(s) is the outermost 1-5, 1-6, 1-7, 1-8, 1-9, 1-10, 1-11, or 1-12 nucleotides. In one embodiment, the 5′ terminal flanking region(s) is the outermost 1-4, 1-5, 1-6, 1-7, or 1-8 nucleotides.

Uniform blocks or stretches of large 2′-sugar modifications within the ASO tend to interfere with the binding of ADAR's dsRNA binding proteins (dsRBDs). Hence, the oligonucleotides disclosed herein may be modified in a way to avoid such interference. For example, the oligonucleotides are modified such that they do not comprise continuous stretches or uniform blocks of nucleotides carrying the same chemical modification (i.e., avoidance of a block-like modification structure). Avoiding uniform blocks of more than 6 nucleotides with the same 2′-modification prevented a strong loss of editing activity with natural ADARs. Hence, in one embodiment, the oligonucleotide is not uniformly modified. In one embodiment, the oligonucleotide contains no uniform blocks and/or no block-like modification structure. In one embodiment, the oligonucleotide does not comprise continuous stretches or uniform blocks of nucleotides carrying the same chemical modification at the 2′ position of the sugar moiety. A “block” or “stretch” may, e.g., not comprise more than 4, 5 or 6 nucleotides with the same 2′-sugar modification. In some instances, the block or stretch may be shorter or longer. In one embodiment, the oligonucleotide contains only 1 block of no more than 6, 5, 4, or 3 nucleotides with the same 2′-sugar modification. In one embodiment, the oligonucleotide contains 2 blocks, separated by one or more oligonucleotides having a different 2′-sugar modification. In order embodiments, the oligonucleotides comprise at least 1 block of nucleotides with the same 2′-sugar modification. In another embodiment, the oligonucleotide comprises 1, 2, 3, or more blocks of nucleotides with the same 2′-sugar modification.

Specifically, stretches of more than 5 nucleotides with the same 2′-modification should be avoided. Avoiding uniform blocks of more than 5 nucleotides with the same 2′-modification prevented a strong loss of editing activity with natural ADARs. Hence, the oligonucleotides disclosed herein may be modified to not include uniform blocks or a continuous stretch of the same 2′-sugar modification. In one embodiment, the oligonucleotide comprises one or more 2′-sugar modifications, optionally wherein no more than 5 consecutive nucleotides have the same 2′-modification. In one embodiment, no more than 4 consecutive nucleotides have the same modification. In one embodiment, no more than 3 consecutive nucleotides have the same modification. In one embodiment, less than 5, 4, or 3 consecutive nucleotides have the same 2′-modification. Hence, in one embodiment, the 2′-sugar modification is 2′-deoxyribose (DNA). In one embodiment, no more than 5 consecutive nucleotides are 2′-H (DNA) modified. In one embodiment, no more than 4 consecutive nucleotides are 2′-H-modified. The 2′-sugar modification may be 2′-ribose. In one embodiment, no more than 6 consecutive nucleotides are 2′-H (DNA) modified. In one embodiment, no more than 5 consecutive nucleotides are 2′-H-modified. In one embodiment, no more than 4 consecutive nucleotides are 2′-H-modified. In one embodiment, no more than 3 consecutive nucleotides are 2′-F-modified. In one embodiment, no more than 2 consecutive nucleotides are 2′-F-modified. In one embodiment, no more than 4 consecutive nucleotides are 2′-O-alkyl-modified, optionally wherein no more than 4 consecutive nucleotides are 2′-OMe-modified.

The oligonucleotide disclosed herein may contain some “continuous stretch(es)” or “uniform block(s)” of a certain length. In one embodiment, the size or length of the “continuous stretch(es)” or “uniform block(s)” is 2, 3, or 4 nucleotides long. In one embodiment, the size or length of the “continuous stretch(es)” or “uniform block(s)” is no more than 2, 3, or 4 nucleotides long. In one embodiment, the oligonucleotide comprises no more than 2 consecutive nucleotides comprising a 2′-F modification. In one embodiment, the oligonucleotide comprises no more than 4 consecutive nucleotides comprising a 2′-OMe modification. In one embodiment, one or more uniform blocks are interrupted. Interruption can take place by any other chemical modification (e.g., DNA, RNA, 2′-F, 2′-OMe, 2′-MOE, LNA, etc.). In one embodiment, one or more uniform blocks of 2′-F-modified nucleotides are interrupted, preferably by 2′-OMe-modified nucleotides. In one embodiment, one or more uniform blocks of 2′-OMe-modified nucleotides are interrupted, preferably by 2′-F-modified nucleotides. In some embodiments the blocks are disrupted by DNA.

DNA oligonucleotides are relatively stable molecules, while RNA oligonucleotides are much more unstable due to their chemical structure. It is commonly known that RNA is subject to autocatalysis and degradation by RNases. To achieve the necessary stability of an oligonucleotide, the final oligonucleotide ideally should not contain any unmodified RNA nucleobases. In one embodiment, the oligonucleotide contains no unmodified RNA nucleobases. In one embodiment, the oligonucleotide contains more than 40%, 50%, 60%, 70%, 80%, or more than 90% modified RNA nucleobases. In one embodiment, the oligonucleotide comprises more than 90% modified RNA nucleobases. In one embodiment, the oligonucleotide contains less than 50%, 40%, 30%, 20%, or less than 10% unmodified RNA nucleobases.

The inventors surprisingly found that depending on the length and symmetry of the ASO, there are preferred combinations of 2′-sugar and internucleoside linkage modifications that improve editing. In particular, the inventors have found that combining 2′-F and/or 2′OMe modifications and sulfonylphosphoramidate internucleoside linkages (e.g., mesyl, esyl, prosyl, busyl, hesyl), preferably mesyl linkages, in the 5′ and 3′ flanking regions improves overall editing when compared to control and ASOs of the prior art. Accordingly, in one embodiment, the oligonucleotide comprises a mixture of 2′-F, 2′OMe and mesyl, esyl, prosyl, busyl, or hesyl linkages.

In one embodiment, the oligonucleotide comprises a mixture of PS, 2′-F, 2′OMe and esyl modifications. In one embodiment, the oligonucleotide comprises a mixture of PS, 2′-F, 2′OMe and prosyl modifications. In one embodiment, the oligonucleotide comprises a mixture of PS, 2′-F, 2′OMe and busyl modifications. In one embodiment, the oligonucleotide comprises a mixture of PS, 2′-F, 2′OMe and hesyl modifications.

In one embodiment, the oligonucleotide comprises a 25-1-8 asymmetry and mesyl, esyl, prosyl, busyl, or hesyl linkages at positions +24, +13, −2, and −8. In one preferred embodiment, the oligonucleotide comprises a 25-1-8 asymmetry and mesyl linkages at positions +24, +13, −2, and −8.

In certain cases, the ASO targeting domain, or nucleobase opposite to the target nucleobase that is to be edited, comprises, one or more wobble bases to compensate for the variability in the target sequence. That is, the less stringent base-pairing requirement of the wobble base (e.g., G-U, I-A, G-A, I-U, I-C, etc.) allows the ASO to pair with more than just one target nucleic acid. Accordingly, in some embodiments, mismatches and/or wobbles enable targeting of different target nucleic acids. In one embodiment, the oligonucleotide comprises one or more additional mismatches, wobble base and/or bulges. In some embodiments, the oligonucleotides disclosed herein may contain bulges of 1, 2, 3 or more nucleotides. In one embodiment, the oligonucleotide comprises one or more mismatches, wobble base, and/or bulges with respect to its target, and/or a mismatch at N₀. In one embodiment, the oligonucleotide comprises one or more mismatches, wobble base, and/or bulges with respect to its target. In one embodiment, the oligonucleotide comprises a mismatch at N₀.

The targeting sequence of the artificial nucleic acid typically comprises a nucleic acid sequence complementary or at least partially complementary to a nucleic acid sequence in the target RNA. In some embodiments, the targeting sequence comprises a nucleic acid sequence complementary or at least 60%, 70%, 80%, 90%, 95% or 99% of a nucleic acid sequence in the target RNA.

The artificial and chemically modified oligonucleotides disclosed herein are suitable for editing a wide variety of endogenous RNA transcripts. For example, the SERPINA1 gene encodes serine protease inhibitor alpha-I antitrypsin (A1AT), which protects tissues from certain inflammatory enzymes, including neutrophil elastase. A deficiency in A1AT (alpha 1 antitrypsin deficiency, A1AD) can lead to excessive break down of elastin in the lungs by neutrophil elastase. This may lead to reduced elasticity in the lungs and subsequent respiratory complications, including emphysema and chronic obstructive lung disease (COPD). Mutant A1AT can also build up in the liver, resulting in cirrhosis and liver failure. Hence, oligonucleotides disclosed herein target SERPINA1.

In one embodiment, the oligonucleotide comprises a nucleobase sequence of 5′-GCCCCAGCAGCTUCAGXCCCUUTCTCNUCGAUGG-3′ (SEQ ID NO: 1); wherein X=thymine (T) or uracil (U); and wherein N=inosine (I) or guanosine (G), preferably deoxyinosine (dI) or deoxyguanosine (dG). In one embodiment, the oligonucleotide comprises a nucleobase sequence of 5′-GCCCCAGCAGCTUCAGUCCCUUTCTCIUCGAUGG-3′ (SEQ ID NO: 2). In one embodiment, the oligonucleotide comprises a nucleobase sequence of 5′-GCCCCAGCAGCTUCAGTCCCUUTCTCIUCGAUGG-3′ (SEQ ID NO: 3).

Also provided herein is a chemically modified oligonucleotide, wherein the oligonucleotide comprises a nucleobase sequence of 5′-GCCCCAGCAGCTUCAGXCCCUUTCTCNUCGAUGG-3′ (SEQ ID NO: 1), wherein X=thymine (T) or uracil (U); wherein N=inosine (I) or guanosine (G); and wherein the oligonucleotide comprises: (i) a mesyl modification; (ii) a PS linkage at position +21 and +4; and (iii) a PO linkage at position +23 and −5. In one embodiment, N is inosine, optionally deoxyinosine. In one embodiment, N is guanosine. In one embodiment, a mesyl linkage is at position +24, +13, −2, and/or −8. In one embodiment, a mesyl linkage is at position +24, +13, −2, and −8.

The oligonucleotides disclosed herein may be modified at their 3′ or 5′ terminus, preferably the 3′ terminus. In one embodiment, the oligonucleotides are modified at their 5′ terminus. In one embodiment, the oligonucleotides are modified at their 3′ terminus. Targeted delivery of oligonucleotides to liver hepatocytes using N-acetylgalactosamine (GalNAc) conjugates has previously described for, e.g., treating liver diseases, including Hepatitis B virus (HBV), non-alcoholic Fatty Liver Disease and genetic diseases (Debacker et al., 2020). Hence, oligonucleotides disclosed herein may comprise a moiety which enhances cellular uptake of the oligonucleotide, e.g., N-acetylgalactosamine (GalNAc). The chemically modified oligonucleotides disclosed herein may comprise a moiety or may be conjugated to a moiety that enhances cellular uptake of the oligonucleotide. In one embodiment, the moiety enhancing cellular uptake is a N-acetyl galactosamine (GalNAc). In one embodiment, GalNAc is conjugated to the 3′ terminus of the oligonucleotide. In one embodiment, the GalNac moiety is conjugated to the 3′ terminus of the oligonucleotide via a C7 linker. In one embodiment, the oligonucleotide comprises a PS linkage or a PO linkage between position −8 and the GalNac moiety. In one embodiment, the linkage between position −8 and the GalNac moiety is a PS linkage.

Certain oligonucleotides disclosed herein specifically target SERPINA1 and share several unifying chemical modifications. However, their modification patterns differ at certain positions. In one embodiment, the oligonucleotide is selected from the group consisting of AI-2811 (SEQ ID NO: 5), AI-2814 (SEQ ID NO: 6), AI-2863 (SEQ ID NO: 7; compound (I)), AI-2864 (SEQ ID NO: 8), AI-2865 (SEQ ID NO: 9), AI-2866 (SEQ ID NO: 10), and AI-3063 (SEQ ID NO: 11). In one embodiment, the oligonucleotide is compound (I).

In one embodiment, the oligonucleotide is AI-1550 (SEQ ID NO: 12).

The inventors have found that the modified oligonucleotides show better stability and editing efficacy. Hence, in one preferred embodiment, the oligonucleotide is AI-2811 (SEQ ID NO: 5). In one embodiment, the oligonucleotide comprises the following sequence from 5′ to 3′:

(SEQ ID NO: 5)
mG&mCmC*fC*fC*mA*fG*mCmoeAfG*fC*moeT&fU*mC*
fA*mGmoeTmoe(MeC)fC*moe(MeC)fU*mUmoeTfC*mo
eT*dC*dl&mU*fC*moeGmAmU*mG&mG(C7)(GalNac),

wherein m=2′-OMe, &=mesyl linkage, *=phosphorothioate, moe=2′-MOE, f=2′-fluoro, I=inosine, d=2′-H (deoxyribose; DNA), MeC=5′-methylcytidine, and A, C, G, T, U=nucleobase.

In one preferred embodiment, the oligonucleotide is AI-2814 (SEQ ID NO: 6). In one embodiment, the oligonucleotide comprises the following sequence from 5′ to 3′: mG&mCmoe(MeC)fC*fC*mA*fG*mCmoeAfG*fC*moeT&fU*moe(MeC)fA*mG*mUmoe(MeC)fC*moe (MeC)fU*mUmoeTfC*moeT*dC*dI&mU*fC*moeGmA*mU*mG&mG (C7) (GalNac) (SEQ ID NO: 6), wherein m=2′-OMe, &=mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

In one preferred embodiment, the oligonucleotide is AI-2863 (SEQ ID NO: 7). In one embodiment, the oligonucleotide comprises the following sequence from 5′ to 3′:

(SEQ ID NO: 7)
mG&mCmC*fC*fC*mA*fG*mCmoeAfG*fC*moeT&mUmC*
fA*mG*mUmoe(MeC)*fC*moe(MeC)fU*mUmoeTfC*mo
eT*dC*dl&mUfC*moeGmA*mU*mG&mG*(C7)(GalNac),

wherein m=2′-OMe, &=mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

In one preferred embodiment, the oligonucleotide is AI-2864 (SEQ ID NO: 8). In one embodiment, the oligonucleotide comprises the following sequence from 5′ to 3′:

(SEQ ID NO: 8)
mG&mCmC*fC*fC*mA*fG*mCmoeAfG*fC*moeT&mUmC*fA*mG*
mUmoe(MeC)*fC*moe(MeC)fU*mUmoeTfC*moeT*dC*dI&mU
fC*moeGfA&mU*mG&mG*(C7)(GalNac),

wherein m=2′-OMe, & =mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

In one preferred embodiment, the oligonucleotide is AI-2865 (SEQ ID NO: 9). In one embodiment, the oligonucleotide comprises the following sequence from 5′ to 3′:

(SEQ ID NO: 9)
mG&mCmC*fC*fC*mA*fG*mCmoeAfG*fC*moeT&mUmC*fA*mG*
mUmoe(MeC)*fC*moe(MeC)fU*mUmoeTfC*moeT*dC*dI&mU
fC*moeGfA*mU*mG&mG*(C7)(GalNac),

wherein m=2′-OMe, & =mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

In one preferred embodiment, the oligonucleotide is AI-2866 (SEQ ID NO: 10). In one embodiment, the oligonucleotide comprises the following sequence from 5′ to 3′:

(SEQ ID NO: 10)
mG&mCmC*fC*fC*mA*fG*mCmoeAfG*fC*moeT&mUmC*fA*mG*
mUmoe(MeC)*fC*moe(MeC)fU*mUmoeTfC*moeT*dC*dI&mU*
fC*moeGfA*mU*mG&mG*(C7)(GalNac),

wherein m=2′-OMe, & =mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

In one preferred embodiment, the oligonucleotide is AI-3063 (SEQ ID NO: 11). In one embodiment, the oligonucleotide comprises the following sequence from 5′ to 3′: mG&mCmoe(MeC)*fC*fC*mA*fG*mCmoeAfG*fC*moeT&mUmoe(MeC)*fA*mG*mUmoe(MeC)*f C*moe(MeC)fU*mUmoeTfC*moeT*dC*dI&mU*fC*moeGfA*mU*mG&moeG*(C7) (GalNac) (SEQ ID NO: 11), wherein m=2′-OMe, & =mesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

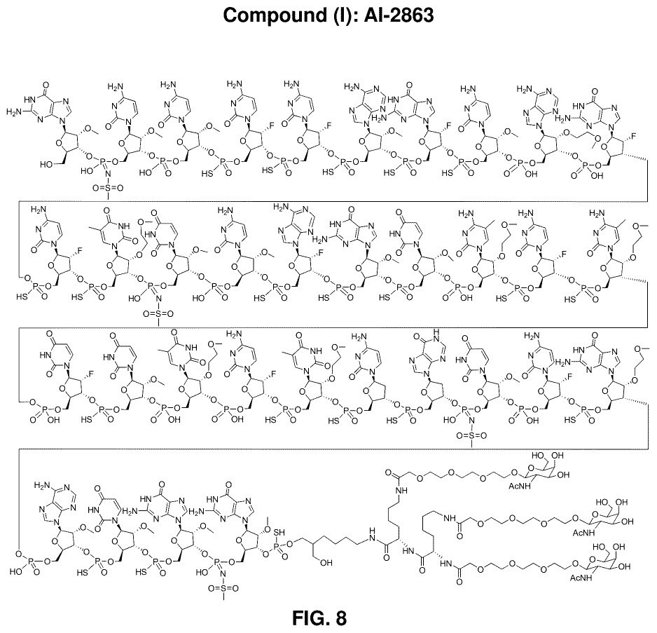

In one embodiment, the oligonucleotide is compound (I). In one embodiment, the oligonucleotide consists of or comprises the formula of compound (I) (see, FIG. 8).

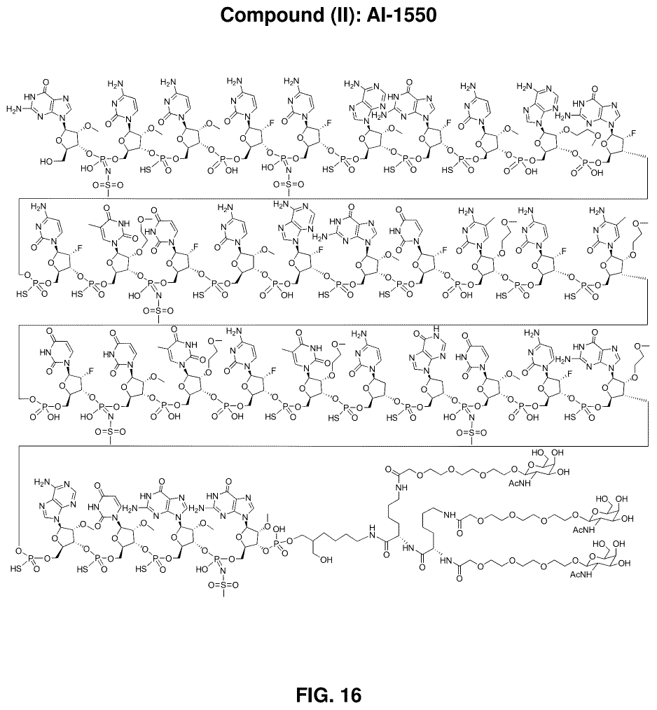

The oligonucleotide may be AI-1550. In one embodiment, the oligonucleotide is compound (II). In one embodiment, the oligonucleotide consists of or comprises the formula of compound (II) (see, FIG. 16).

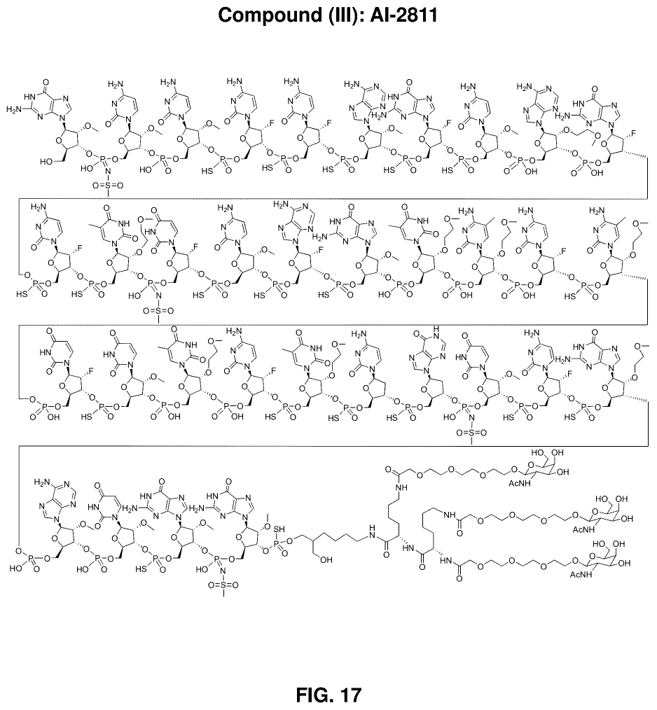

In one embodiment, the oligonucleotide is AI-2811. In one embodiment, the oligonucleotide is compound (III). In one embodiment, the oligonucleotide consists of or comprises the formula of compound (III) (see, FIG. 17).

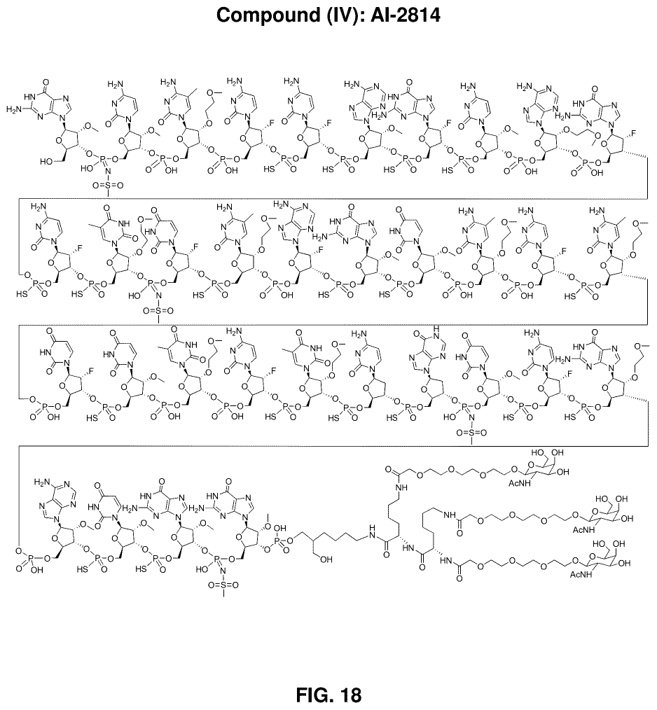

In one embodiment, the oligonucleotide is AI-2814. In one embodiment, the oligonucleotide is compound (IV). In one embodiment, the oligonucleotide consists of or comprises the formula of compound (IV) (see, FIG. 18).

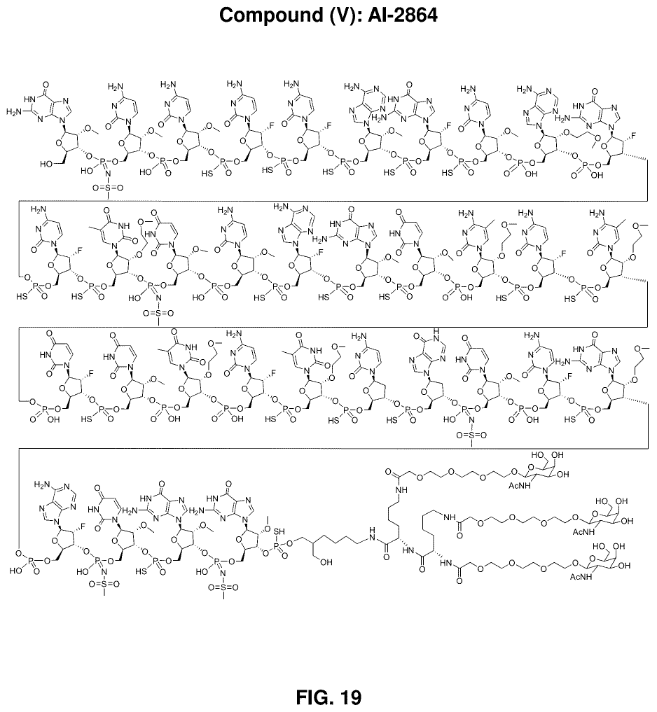

In one embodiment, the oligonucleotide is AI-2864. In one embodiment, the oligonucleotide is compound (V). In one embodiment, the oligonucleotide consists of or comprises the formula of compound (V) (see, FIG. 19).

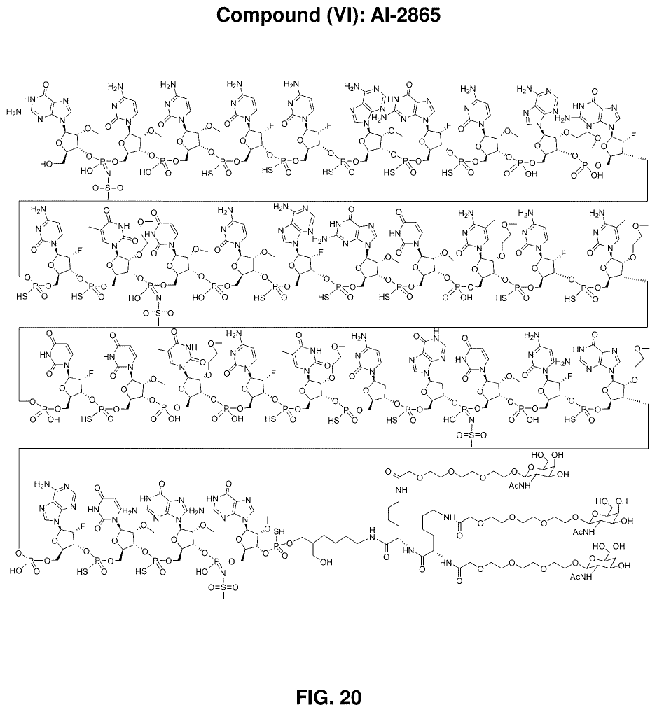

In one embodiment, the oligonucleotide is AI-2865. In one embodiment, the oligonucleotide is compound (VI). In one embodiment, the oligonucleotide consists of or comprises the formula of compound (VI) (see, FIG. 20).

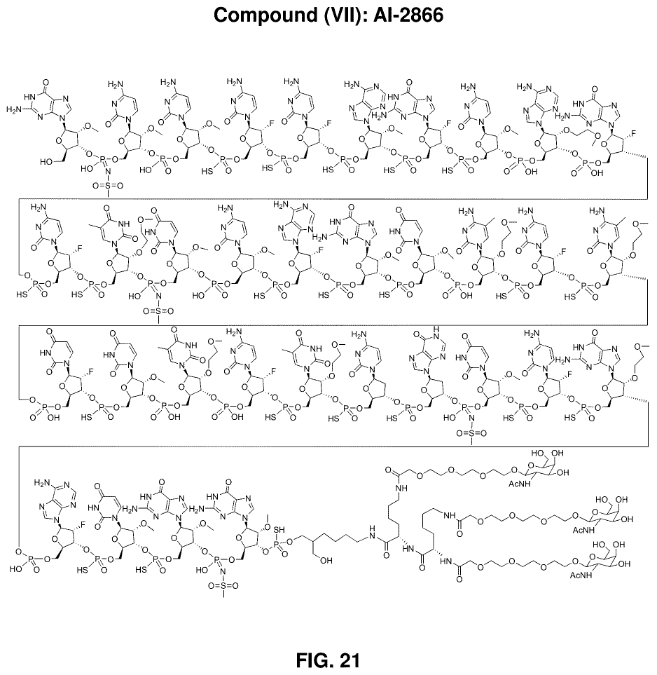

In one embodiment, the oligonucleotide is AI-2866. In one embodiment, the oligonucleotide is compound (VII). In one embodiment, the oligonucleotide consists of or comprises the formula of compound (VII) (see, FIG. 21).

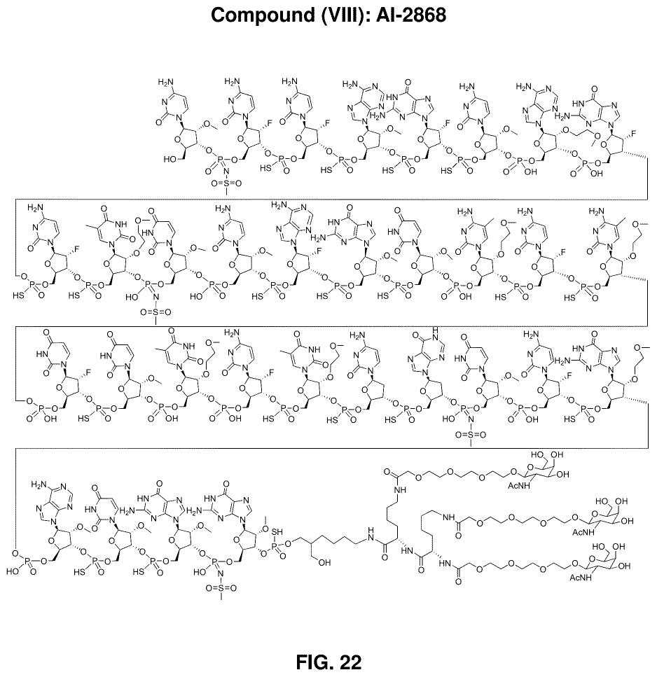

In one embodiment, the oligonucleotide is AI-2868. In one embodiment, the oligonucleotide is compound (VIII). In one embodiment, the oligonucleotide consists of or comprises the formula of compound (VIII) (see, FIG. 22).

In one embodiment, the oligonucleotide comprises or consists of AI-4486 (SEQ ID NO: 21). In one embodiment, the oligonucleotide is AI-4486 (SEQ ID NO: 21). In one embodiment, the oligonucleotide comprises or consists of the following sequence from 5′ to 3′: mG[>e]mCmC*fC*fC*mA*fG*mCmoeAfG*fC*moeT[>e]mUmC*fA*mG*mUmoe(MeC)*fC*moe(M eC)fU*mUmoeTfC*moeT*dC*dI[>e]mUfC*moeGmA*mU*mG [>e]mG*(C7) (GalNac), wherein m=2′-OMe, [>e]=esyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

In one embodiment, the oligonucleotide comprises or consists of AI-4487 (SEQ ID NO: 22). In one embodiment, the oligonucleotide is AI-4487 (SEQ ID NO: 22). In one embodiment, the oligonucleotide comprises or consists of the following sequence from 5′ to 3′: mG[>p]mCmC*fC*fC*mA*fG*mCmoeAfG*fC*moeT[>p]mUmC*fA*mG*mUmoe(MeC)*fC*moe(M eC)fU*mUmoeTfC*moeT*dC*dI[>p]mUfC*moeGmA*mU*mG[>p]mG*(C7)(GalNac), wherein m=2′-OMe, [>p]=prosyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

In one embodiment, the oligonucleotide comprises or consists of AI-4488 (SEQ ID NO: 23). In one embodiment, the oligonucleotide is AI-4488 (SEQ ID NO: 23). In one embodiment, the oligonucleotide comprises or consists of the following sequence from 5′ to 3′: mG[>b]mCmC*fC*fC*mA*fG*mCmoeAfG*fC*moeT[>b]mUmC*fA*mG*mUmoe(MeC)*fC*moe (M eC)fU*mUmoeTfC*moeT*dC*dI[>b]mUfC*moeGmA*mUmG[>b]mG*(C7) (GalNac), wherein m=2′-OMe, [>b]=busyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

In one embodiment, the oligonucleotide comprises or consists of AI-4543 (SEQ ID NO: 24). In one embodiment, the oligonucleotide is AI-4543 (SEQ ID NO: 24). In one embodiment, the oligonucleotide comprises or consists of the following sequence from 5′ to 3′: mG[>h]mCmC*fC*fC*mA*fG*mCmoeAfG*fC*moeT[>h]mUmC*fA*mG*mUmoe(MeC)*fC*moe (M eC)fU*mUmoeTfC*moeT*dC*dI[>h]mUfC*moeGmA*mU*mG [>h]mG*(C7) (GalNac), wherein m=2′-OMe, [>h]=hesyl linkage, *=phosphorothioate, moe=2′-MOE, f=2′-fluoro, MeC=5′-methylcytidine, I=inosine, d=2′-H (deoxyribose; DNA), and A, C, G, T, U=nucleobase.

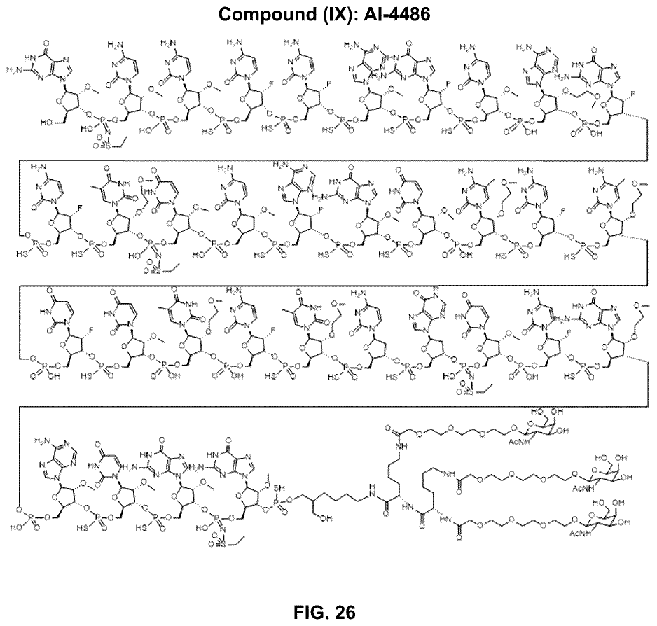

In one embodiment, the oligonucleotide is AI-4486. In one embodiment, the oligonucleotide is compound (IX). In one embodiment, the oligonucleotide consists of or comprises the formula of compound (IX) (see, FIG. 26).

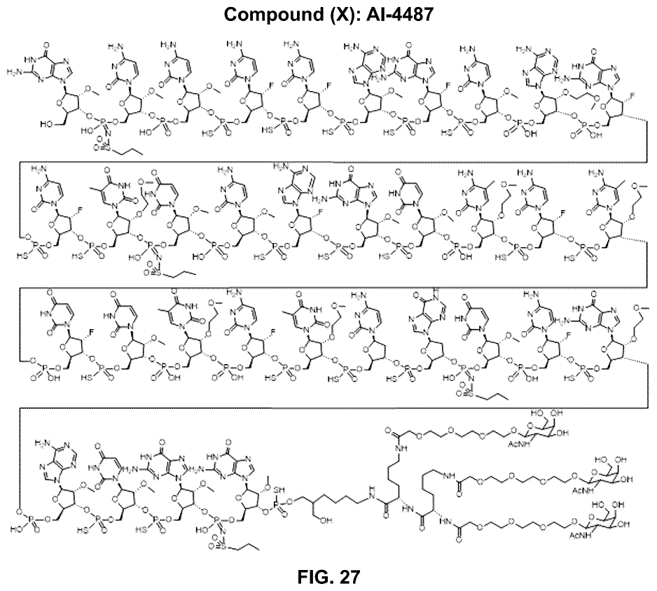

In one embodiment, the oligonucleotide is AI-4487. In one embodiment, the oligonucleotide is compound (X). In one embodiment, the oligonucleotide consists of or comprises the formula of compound (X) (see, FIG. 27).

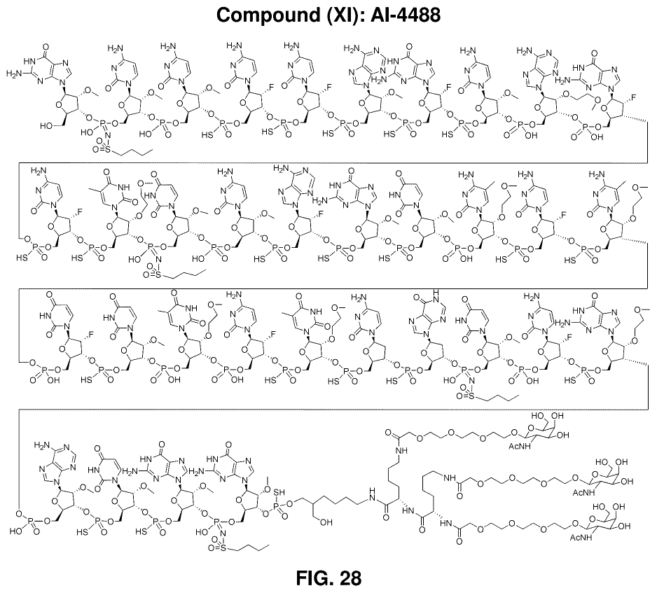

In one embodiment, the oligonucleotide is AI-4488. In one embodiment, the oligonucleotide is compound (XI). In one embodiment, the oligonucleotide consists of or comprises the formula of compound (XI) (see, FIG. 28).

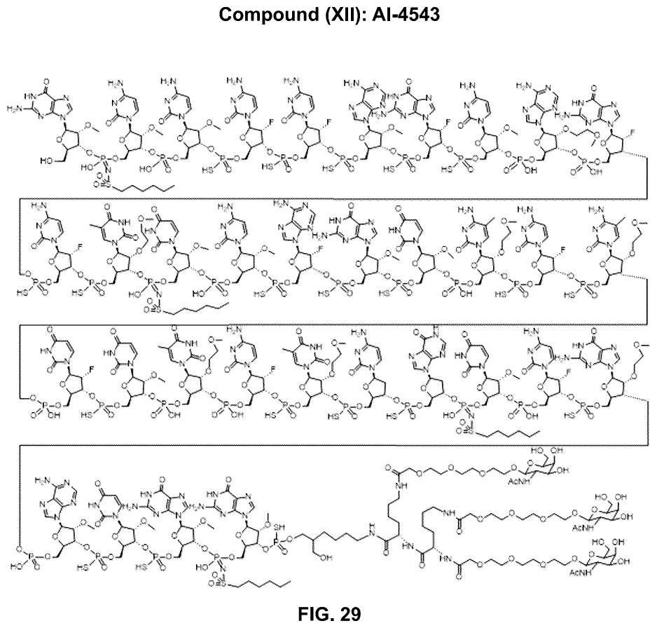

In one embodiment, the oligonucleotide is AI-4543. In one embodiment, the oligonucleotide is compound (XII). In one embodiment, the oligonucleotide consists of or comprises the formula of compound (XII) (see, FIG. 29).

The chemically modified oligonucleotides according to the present disclosure show increased hydrophobicity, stability against degradation and an optimal chemical modification pattern to bind ADARs. The oligonucleotides according to the disclosure differ from the nucleic acid oligonucleotides disclosed in the prior art insofar that they do not require a loop-hairpin structured recruiting moiety specifically for recruiting a deaminase. The oligonucleotides of the present disclosure may or may not comprise a loop-hairpin structure. In one embodiment, the chemically modified oligonucleotide does not comprise a loop-hairpin structured recruiting moiety.

### Compositions

The chemically modified oligonucleotides disclosed herein may be incorporated into compositions. Accordingly, provided herein is a composition containing the oligonucleotide(s) disclosed herein. In some embodiments, the compositions are pharmaceutical compositions. In the context disclosed herein, the term composition and pharmaceutic compositions are used interchangeably. Hence, in some embodiments, the present disclosure provides oligonucleotide compositions of oligonucleotides described herein. In one embodiment, the composition contains one or more oligonucleotides disclosed herein. In some embodiments, the present disclosure provides a composition comprising a plurality of oligonucleotides. As used herein, pharmaceutical composition means a substance or a mixture of substances suitable for administering to an individual. For example, a pharmaceutical composition may comprise one or more active pharmaceutical agents (such as an oligonucleotide) and a sterile aqueous solution. The compositions provided herein can be in any form that allows for the composition to be administered to a subject. The compositions may be used in methods of treating and/or preventing a genetic disorder, condition, or disease.

In one embodiment, the composition comprises an oligonucleotide disclosed herein or a pharmaceutically acceptable salt thereof. In one embodiment, a composition comprises an oligonucleotide disclosed herein in an admixture with a pharmaceutically acceptable carrier. In some embodiments, the pharmaceutically acceptable carrier can simply be a saline solution. This can be isotonic or hypotonic.

In one embodiment, the composition is for veterinary and/or human administration. In some embodiments, a pharmaceutical composition comprises one or more other therapies in addition to an oligonucleotide disclosed herein.

The amount of an oligonucleotide or composition which will be effective in the treatment and/or prevention of a disease or disorder will depend on the nature of the disease and can be determined by standard clinical techniques. Exemplary doses for oligonucleotides range from about 10 ng to 1 g, 100 ng to 100 mg, 1 μg to 10 mg, or 30-300 μg oligonucleotide, e.g., RNA, per patient. In one embodiment, the oligonucleotide is present at a concentration of 4 nM to 100 nM, optionally at 20 nM or 25 nM. In one embodiment, the oligonucleotide is present at a concentration of 0.8 nM. In one embodiment, the oligonucleotide is present at a concentration of 4 nM. In one embodiment, the oligonucleotide is present at a concentration of 20 nM. In one embodiment, the oligonucleotide is present at a concentration of 25 nM.

In certain embodiments, the compositions disclosed herein include diluents of various buffer content (e.g., Tris-HCl, acetate, phosphate), pH, and ionic strength, and additives such as detergents and solubilizing agents (e.g., Tween 80, Polysorbate 80), anti-oxidants (e.g., ascorbic acid, sodium metabisulfite), preservatives (e.g., Thimersol, benzyl alcohol), and bulking substances (e.g., lactose, mannitol). In some embodiments, the material is incorporated into particulate preparations of polymeric compounds such as polylactic acid, polyglycolic acid, etc. or into liposomes. In some embodiments, hyaluronic acid is used. Such compositions may influence the physical state, stability, rate of in vivo release, and/or rate of in vivo clearance of the oligonucleotides and/or derivatives and/or pharmaceutically acceptable salt thereof. In some embodiments, the compositions are in liquid form or in dried powder, such as lyophilized form.

In certain embodiments, the compositions additionally comprise one or more salts, e.g., sodium chloride, calcium chloride, sodium phosphate, monosodium glutamate, and aluminium salts (e.g., aluminium hydroxide, aluminium phosphate, alum (potassium aluminium sulfate), or a mixture of such aluminium salts). In other embodiments, the compositions described herein do not comprise salts.

The pharmaceutical compositions can be included in a container, pack, or dispenser together with instructions for administration.

The oligonucleotides or compositions thereof can be tested for in vivo toxicity in animal models. For example, animal models, described herein and/or others known in the art, used to test the activities of active compounds can also be used to determine the in vivo toxicity of these compounds. For example, animals are administered a range of concentrations of active compounds. Subsequently, the animals are monitored over time for lethality, weight loss or failure to gain weight, and/or levels of serum markers that may be indicative of tissue damage. These in vivo assays may also be adapted to test the toxicity of various administration mode and/or regimen in addition to dosages.

### Prophylactic and Therapeutic Uses

In further aspects, the present disclosure describes the use of chemically modified oligonucleotides and compositions comprising the same in the medical setting, specifically, for site-directed editing of a target RNA (e.g., binding to the target RNA via the targeting sequence and by recruiting to the target site a deaminase). The disclosure describes chemically modified oligonucleotides and compositions comprising said oligonucleotides for use in the treatment or prevention of a genetic disorder, condition, or disease as well as methods for treating or preventing a genetic disorder, condition, or disease. Site-directed editing may take place in vitro, in vivo or ex vivo.

A chemically modified oligonucleotide or composition comprising the same may be used in the treatment and/or prevention of a medical condition. In one aspect provided herein is a chemically modified oligonucleotide disclosed herein or a composition disclosed herein for use in therapy. In another aspect provided herein is an oligonucleotide disclosed herein or a composition comprising the same for use in the treatment or prevention of a genetic disorder, condition, or disease. In some embodiments, the disease or disorder is selected form the group consisting of liver or metabolic diseases and/or cardiac or cardiovascular diseases associated with a gain-of-function (GOF) or loss-of-function (LOF) mutation. In one embodiment, the disease or disorder is associated with a point mutation. For example, the SERPINA1 gene encodes serine protease inhibitor alpha-I antitrypsin (A1AT). A1AT protects tissues from certain inflammatory enzymes, including neutrophil elastase. A deficiency in A1AT (alpha 1 antitrypsin deficiency, A1AD) can lead to excessive break down of elastin in the lungs by neutrophil elastase. This may lead to reduced elasticity in the lungs and subsequent respiratory complications, including emphysema and chronic obstructive lung disease (COPD). Mutant A1AT can also build up in the liver, resulting in cirrhosis and liver failure. Accordingly, in one embodiment, the disease or disorder is associated with a G-to-A mutation in the SERPINA1 gene. In one embodiment, the mutation is selected from SERPINA1 E342K. In one embodiment, the disease or disorder comprises the SERPINA1 gene or an alpha-1-antitrypsin deficiency (A1AD or AATD), optionally wherein the target protein is alpha-1 antitrypsin. In one embodiment, the mutation is the PiZ mutation (α1-antitrypsin deficiency). In one embodiment, the treatment leads to normal (wildtype) alpha-1 antitrypsin (AAT). In one embodiment, the treatment leads to improved alpha-1 antitrypsin (AAT) protein expression.

Also, a chemically modified oligonucleotide disclosed herein or a pharmaceutical composition may be used in the diagnosis of a genetic condition, disease or disorder. Therein, the disease or disorder is preferably selected from the group consisting of infectious diseases, tumour diseases, cardiovascular diseases, autoimmune diseases, allergies and neurological diseases or disorders. Hence, in one embodiment, the disease or disorder is an infectious disease. In one embodiment, the disease or disorder is a tumour or cancer disease. In one embodiment, the disease or disorder is a cardiovascular disease. In one embodiment, the disease or disorder is an autoimmune disease. In one embodiment, the disease or disorder is an allergy. In one embodiment, the disease or disorder is a neurological disease or disorder. In one embodiment, the genetic disorder, condition or disease is associated with a G-to-A mutation.

The chemically modified oligonucleotide disclosed herein or the (pharmaceutical) composition may be administered, for example, orally in any orally acceptable dosage form including, but not limited to, capsules, tablets, aqueous suspensions, or solutions, or parenterally, e.g., by parenteral injection. In some embodiments, formulations suitable for parenteral administration comprise sterile aqueous preparations of at least one embodiment of the present disclosure, which are approximately isotonic with the blood of the intended recipient. The amount of oligonucleotide or composition to be administered, the dosage and the dosing regimen can vary from cell type to cell type, the disease to be treated, the target population, the mode of administration (e.g., systemic versus local), the severity of disease and the acceptable level of side activity. In some embodiments, the amount of oligonucleotides administered in a pharmaceutical composition is dependent on the subject being treated, the subject's weight, the manner of administration.

Various delivery systems can be used to deliver the oligonucleotides disclosed herein. An oligonucleotide according to the present disclosure can be delivered as is, i.e., naked and/or in isolated form to an individual, through an organ, e.g., mucosa of the eye, or directly to a cell. Hence, in a preferred embodiment, the oligonucleotide disclosed herein is administered and delivered ‘as is’, also referred to as ‘naked’. When administering an oligonucleotide disclosed herein, it is preferred that the oligonucleotide is dissolved in a solution that is compatible with the delivery method. Such delivery may be in vivo, in vitro or ex vivo. Hence, depending on the disease, disorder or infection that needs to be treated, or on the cell, tissue or part of the body that needs to be reached by the oligonucleotides (e.g., in case of beneficial editing), a different administration route or delivery method may be selected. Examples for delivery when an oligonucleotide is not delivered naked, are delivery agents or vehicles such as nanoparticles, like polymeric nanoparticles, microparticles, micelles, liposomes, antibody-conjugated liposomes, cationic lipids, polymers, or cell-penetrating peptides.

Use of an excipient or transfection reagents may be used in the delivery of each of the oligonucleotides or compositions to a cell and/or into a cell (preferably a cell affected by a G-to-A mutation or that wherein “beneficial editing” is to be achieved as outlined herein). Preferred are excipients or transfection reagents capable of forming complexes, nanoparticles, micelles, vesicles and/or liposomes that deliver each oligonucleotide or composition as defined herein, complexed or trapped in a vesicle or liposome through a cell membrane. Many of these excipients are known in the art. Suitable excipients or transfection reagents comprise polyethylenimine (PEI; ExGen500 (MBI Fermentas)), LipofectAMINE™ 2000 (Invitrogen), Lipofectin™, or derivatives thereof, and/or viral capsid proteins that are capable of self-assembly into particles that can deliver each constituent as defined herein to a target cell.

Oligonucleotides disclosed herein may be linked to a moiety that enhances uptake of the ASO in cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment, or a single chain antigen binding domain such as a cameloid single domain antigen-binding domain. Accordingly in some embodiments, the ASO is delivered using drug conjugates with antibodies, nanobodies, cell penetrating peptides and aptamers. In one embodiment, the oligonucleotide is conjugated to an antibody, preferably a Fab fragment.

The oligonucleotide or composition may be administered as a monotherapy or in combination with a further different medicament, particularly a medicament suitable for the treatment or prevention of alpha-1-antitrypsin (A1AT) deficiency.

Compared to reference oligonucleotides or compositions, the provided oligonucleotides or compositions are surprisingly effective. In some embodiments, a change is measured by an increase of a desired mRNA and/or protein level compared to a reference sample or condition. In some embodiments, a change is measured by an increase in the editing efficacy (%) mediated by the oligonucleotide or composition comprising the same disclosed herein. In some embodiments, a change is measured by an increase in stability of the oligonucleotide or composition comprising the same. In some embodiments, a change is measured in the levels of cytotoxicity, viability, apoptosis or immune activation. In some embodiments, a change is detected by means of luminescence and/or gene expression. In some embodiments, toxicity and therapeutic efficacy can be determined by standard pharmaceutical procedures in cell cultures or experimental animals, e.g., for determining the LD₅₀ (the dose lethal to 50% of the population) and the LD₅₀ (the dose therapeutically effective in 50% of the population). In some embodiments, data obtained from the cell culture assays or animal studies can be used in formulating a range of dosage for use in humans.

Further provided herein is an in vitro method for site-directed A-to-I editing of a target RNA, the method comprising a step of contacting a target RNA with the chemically modified oligonucleotide disclosed herein or the composition disclosed herein. For instance, the method may be for beneficial and/or compensatory RNA editing. That is, the method may be for targeting wildtype adenosines for beneficial editing or for targeting wildtype adenosines for compensatory editing.

The compositions of the present disclosure can be administered in a number of ways depending upon whether local or systemic treatment is desired and upon the area to be treated. Administration may be by inhalation (e.g., through nebulization), intranasally, orally, by injection or infusion, intravenously, subcutaneously, intra-dermally, intra-cranially, intramuscularly, intra-tracheally, intra-peritoneally, intra-rectally, by direct injection into a tumour, and the like. Administration may be in solid form, in the form of a powder, a pill, or in any other form compatible with pharmaceutical use in humans. In some embodiments the oligonucleotide construct can be delivered systemically.

Also provided herein is a method for treating a subject suffering from a disease or disorder. In some embodiments, the disclosure provides a method of treating a subject having a disease or disorder. In some embodiments, the disclosure provides a method of treating a subject in need thereof. In some embodiments, the method comprises administering an effective amount of a chemically modified oligonucleotide or a composition disclosed herein to the subject. In one embodiment, the genetic disease or disorder is a liver or metabolic diseases and/or cardiac or cardiovascular diseases, which can be treated with or is associated with a gain-of-function (GOF) or loss-of-function (LOF) mutation, optionally wherein the disease or disorder is associated with the SERPINA1 gene or an alpha-1-antitrypsin deficiency (A1AD or AATD). In some embodiments, the disease or disorder is associated with the SERPINA1 gene or an alpha-1-antitrypsin deficiency (A1AD or AATD).

Also provided herein is the use of an oligonucleotide disclosed herein or composition disclosed herein in the manufacture of a medicament for the treatment of a subject suffering from a genetic disease or disorder, wherein the genetic disease or disorder is a liver or metabolic diseases and/or cardiac or cardiovascular diseases, which can be treated with or is associated with a gain-of-function (GOF) or loss-of-function (LOF) mutation, optionally wherein the disease or disorder is associated with the SERPINA1 gene or an alpha-1-antitrypsin deficiency (A1AD or AATD). In one embodiment, the disease or disorder is associated with the SERPINA1 gene or an alpha-1-antitrypsin deficiency (A1AD or AATD) and wherein the mutation is SERPINA1 E342K.

### Patient Population

The oligonucleotides disclosed herein of compositions comprising the same may be administered to various groups of subjects or patients. In certain embodiments, the patient is in need of treatment. In other embodiments, the patient is not in need of treatment (“beneficial editing”). That is, the subject receives the oligonucleotide or composition to edit an RNA derived from a wildtype allele (not a mutated allele) in order to modulate the function of the wildtype protein in a useful way.

In one embodiment, an oligonucleotide or composition containing the same is administered to a subject. In some embodiments, an oligonucleotide or composition containing the same is administered to a mammal, preferably a human. In certain embodiments, an oligonucleotide or composition containing the same is administered to a naive subject, i.e., a subject that does not have a disease or disorder. In one embodiment, an oligonucleotide or composition containing the same is administered to a naive subject that is at risk of developing a disease or disorder. In some embodiments, an oligonucleotide or composition containing the same is administered to a patient before symptoms manifest or symptoms become severe. In certain embodiments, an oligonucleotide or composition containing the same is administered to a patient who has been diagnosed with a disease or disorder.

In some embodiments, the subject to be administered an oligonucleotide or composition containing the same is any individual at risk of developing a disease or disorder associated with a G-to-A mutation. In one embodiment, the mutation is within the SERPINA1 gene. In one embodiment, the subject suffers from a disease or disorder associated with a G-to-A mutation in genes, preferably within the SERPINA1 gene. In some embodiments, a symptom of a condition, disorder or disease associated with a G-to-A mutation can be any condition, disorder or disease that can benefit from an A-to-I conversion within the target RNA of the particular gene.

Also provided herein are methods of treating a subject suffering from a disease or disorder, comprising administering an effective amount of the chemically modified oligonucleotide disclosed herein or the composition disclosed herein. In one embodiment, the genetic disease or genetic disorder is associated with a G-to-A mutation in a subject. Also provided herein is a method for treating a subject suffering from a genetic disease or genetic disorder, comprising administering an effective amount of the chemically modified oligonucleotide disclosed herein or the composition disclosed herein. In one embodiment, the disease or disorder is a liver or metabolic disease and/or a cardiac or cardiovascular disease which can be treated with or is associated with a gain-of-function (GOF) or loss-of-function (LOF) mutation. In one embodiment, the disease or disorder is associated with the SERPINA1 gene.

In some embodiments, the methods described herein lead to improved symptoms in the subject. In some embodiments, administering the chemically modified oligonucleotide leads to a reduction (e.g., at least 5%, at least 10%, at least 20%, at least 30%, at least 50%, at least 75%, or at least 100% reduction) in liver inflammation relative to liver inflammation in the subject prior to administering an effective amount of the chemically modified oligonucleotide. In some embodiments, administering the chemically modified oligonucleotide leads to a reduction (e.g., at least 5%, at least 10%, at least 20%, at least 30%, at least 50%, at least 75%, or at least 100% reduction) in the number of inflammatory foci in the liver relative to the number of inflammatory foci in the liver prior to administering an effective amount of the chemically modified oligonucleotide. In some embodiments, administering the chemically modified oligonucleotide leads to a reduction (e.g., at least 5%, at least 10%, at least 20%, at least 30%, at least 50%, at least 75%, or at least 100% reduction) in the density of large Z-AAT globules relative to the density of large Z-AAT globules prior to administering an effective amount of the chemically modified oligonucleotide. In some embodiments, administering the chemically modified oligonucleotide leads to a reduction (e.g., at least 5%, at least 10%, at least 20%, at least 30%, at least 50%, at least 75%, or at least 100% reduction) in (i) liver inflammation, (ii) the number of inflammatory foci in the liver, and/or (iii) the density of large Z-AAT globules, relative to (i)-(iii) in the subject prior to administering an effective amount of the chemically modified oligonucleotide. In some embodiments, the subject has a liver injury caused by Z-AAT accumulation. In some embodiments, the liver injury improves (i.e., is less injured) after administering an effective amount of the chemically modified oligonucleotide.

In some embodiments, the methods described herein, comprising administering any one of the chemically modified oligonucleotides described herein, lead to an increase (e.g., at least 5%, at least 10%, at least 20%, at least 30%, at least 50%, at least 75%, at least 100%, at least 2-fold, at least 5-fold, at least 10-fold, at least 25-fold, at least 50-fold, at least 75-fold, or at least 100-fold increase) in M-AAT serum levels and/or total AAT serum levels. In some embodiments, the method leads to an increase (e.g., at least 5%, at least 10%, at least 20%, at least 30%, at least 50%, at least 75%, at least 100%, at least 2-fold, at least 5-fold, at least 10-fold, at least 25-fold, at least 50-fold, at least 75-fold, or at least 100-fold increase) in M-AAT serum levels. In some embodiments, the method leads to an increase (e.g., at least 5%, at least 10%, at least 20%, at least 30%, at least 50%, at least 75%, at least 100%, at least 2-fold, at least 5-fold, at least 10-fold, at least 25-fold, at least 50-fold, at least 75-fold, or at least 100-fold increase) in total AAT serum levels.

Also provided herein is the use of an oligonucleotide disclosed herein in therapy. Also, provided herein is a use of an oligonucleotide disclosed herein in the manufacture of a medicament for treating conditions, diseases and/or disorders associated with a G-to-A mutation. Also provided herein is the use of an oligonucleotide disclosed herein in the manufacture of a medicament for treating or preventing a (genetic) disease or disorder associated with a G-to-A mutation. In certain embodiments, the use of an oligonucleotide disclosed herein is in the manufacture of a medicament for treating a disease or disorder associated with the SERPINA1 gene or an alpha-1-antitrypsin deficiency (A1AD or AATD).

The composition disclosed herein comprises the oligonucleotide disclosed herein. According to a further aspect, the disclosure relates to a kit or kit of parts comprising an oligonucleotide disclosed herein and/or the (pharmaceutical) composition disclosed herein. The kit additionally comprises instructions for use.

### Methods for Editing

The present disclosure also relates to methods for editing a target adenosine in a target nucleic acid. Specifically, the present disclosure provides methods of editing a SERPINA1 polynucleotide, e.g., a SERPINA1 polynucleotide comprising a single nucleotide polymorphism (SNP) associated with alpha I antitrypsin deficiency (A1AD or AATD). Hence, the present disclosure provides a method for site-directed A-to-I editing of a target RNA, the method comprising a step of contacting a target RNA with the chemically modified oligonucleotide disclosed herein or the composition disclosed herein. In one embodiment, the method comprises, after the step of contacting, the following steps: (a) allowing uptake by the cell of the chemically modified oligonucleotide; (b) allowing annealing of the chemically modified oligonucleotide to the target RNA; and (c) allowing a mammalian ADAR enzyme comprising a natural dsRNA binding domain as found in the wildtype enzyme to deaminate the target adenosine in the target RNA sequence to an inosine. In one embodiment, the target RNA is derived from the SERPINA1 gene.

The present disclosure also provides an in vitro method for deaminating at least one specific adenosine present in a target RNA sequence in a cell. Hence, in on aspect provided herein is an in vitro method for deaminating at least one specific adenosine present in a target RNA sequence in a cell, wherein the method comprises the steps of: (a) contacting the target nucleic acid with a chemically modified oligonucleotide disclosed herein; (b) allowing uptake by the cell of the chemically modified oligonucleotide; (c) allowing annealing of the chemically modified oligonucleotide to the target RNA sequence; and (d) allowing a mammalian ADAR enzyme comprising a natural dsRNA binding domain as found in the wild type enzyme to deaminate the target adenosine in the target RNA sequence to an inosine.

In one embodiment, the method comprises after step (d), a step (e) of identifying the presence of the inosine in the RNA sequence.

The editing reaction is preferably monitored or controlled by sequence analysis of the target RNA.

Also, a chemically modified oligonucleotide disclosed herein or a (pharmaceutical) composition may be used in the diagnosis of a disease or disorder or a genetic condition. Herein, the disease or disorder is preferably selected from the group consisting of infectious diseases, tumour diseases, cardiovascular diseases, autoimmune diseases, allergies and neurological diseases or disorders. In one embodiment, the disease or disorder is selected form the group consisting of liver, metabolic, neurodegenerative and cardiac or cardiovascular diseases or disorders. In one embodiment, the disease or disorder is associated with a G-to-A mutation. In one embodiment, the disease or disorder can be treated with a gain-of-function (GOF) mutation in SERPINA1. That is, in one embodiment, the GOF leads to normal (wildtype) alpha-1 antitrypsin (AAT) protein expression. In one embodiment, the GOF leads to improved alpha-1 antitrypsin (AAT) protein expression.

Compositions and methods provided in the present disclosure can be used to make desired changes in a target sequence in a cell or a subject by site-directed editing of nucleotides using an oligonucleotide that is capable of effecting an adenosine deaminase acting on RNA (ADAR)-mediated adenosine to inosine. As a result, the target sequence is edited through an adenosine deamination reaction mediated by ADAR, converting adenosines into inosine. In some embodiments, because I is recognized as G, the deamination correcting the pathogenic mutation in the SERPINA1 gene reverses the E342K mutation back to wild-type, reversing or slowing symptoms associated with A1AD experienced by the patient.

The methods of the present disclosure can be used with cells from any organ, e.g., skin, lung, heart, kidney, liver, pancreas, gut, muscle, gland, eye, brain, blood and the like. Compositions and methods of the present disclosure are particularly suitable for modifying sequences in cells, tissues or organs implicated in a diseased state of a (human) subject. For example, such cells may include, but are not limited, to hepatocytes, hepatocyte-like cells, and/or alveolar type II cells, neurons (PNS, CNS), retina, photo receptors cells, Müller Glia cells, RPE, immune cells, B cells, T cells, dendritic cells, macrophages.

### LIST OF FURTHER EMBODIMENTS

Aspects of the present disclosure are further described by the following non-limiting embodiments:

In one embodiment, oligonucleotides disclosed herein comprise or consist of one or more esyl linkages.

In one embodiment, oligonucleotides disclosed herein comprise or consists of esyl linkages at position +24, +13, −2 and −8.

In one embodiment, the oligonucleotide comprises or consists of AI-4486 (SEQ ID NO: 21).

In one embodiment, oligonucleotides disclosed herein comprise or consist of one or more prosyl linkages.

In one embodiment, oligonucleotides disclosed herein comprise or consists of prosyl linkages at position +24, +13, −2 and −8.

In one embodiment, the oligonucleotide comprises or consists of AI-4487 (SEQ ID NO: 22).

In one embodiment, oligonucleotides disclosed herein comprise or consist of one or more busyl linkages.

In one embodiment, oligonucleotides disclosed herein comprise or consists of busyl linkages at position +24, +13, −2 and −8.

In one embodiment, the oligonucleotide comprises or consists of AI-4488 (SEQ ID NO: 23).

In one embodiment, oligonucleotides disclosed herein comprise or consist of one or more hesyl linkages.

In one embodiment, oligonucleotides disclosed herein comprise or consists of hesyl linkages at position +24, +13, −2 and −8.

In one embodiment, the oligonucleotide comprises or consists of or AI-4543 (SEQ ID NO:24).

In one embodiment, oligonucleotides disclosed herein comprise or consist of one or more tosyl linkages.

In one embodiment, oligonucleotides disclosed herein comprise or consists of tosyl linkages at position +24, +13, −2 and −8.

### EXAMPLES

Compositions and methods of the present disclosure shall be described in more detail by the following Examples. The examples shown in the following are merely illustrative and shall describe the aspects of the present disclosure in a further way. These examples shall not be construed to limit any aspects of the present disclosure thereto. The sequences disclosed herein are also shown in the enclosed sequence listing. However, the sequence listing shows only the sequence of nucleotides, whereas the modification of the nucleotides and of the bonds between the nucleotides is not shown in the sequence listing. The relevant modifications associated with the sequences are disclosed in the tables below and, to some extent, in the Figures of this application.

For all experiments, target editing efficacy is expressed as the percentage [%] of edited target sites found in all detected target sites in the target transcript.

### General Protocol:

Oligonucleotide synthesis: Generally, oligonucleotides were synthesized DMT-ON on a 200 nmol scale using 1000 Å CPG supports from Glen Research: either standard or universal (loading of ca. 30 μmol/g) on a MerMade48 oligonucleotide synthesizer. Fully protected nucleoside phosphoramidites were incorporated using standard solid-phase oligonucleotide synthesis, i.e. 3% dichloroacetic acid in DCM for deblocking, 0.25 M ETT in acetonitrile as activator for amidite couplings, 20% acetic anhydride in THF and 10% 1-methylimidazole in THF/pyridine for capping, 0.02M iodine in THF/water/pyridine for oxidation and 0.1 M xanthane hydride in pyridine: acetonitrile 1:1 (v:v) for thiolation. Mesyl phosphoramidate linkages were obtained via Staudinger reaction, which was carried out with 0.5 M solution of mesyl azide (Aurum Pharmatech) in dry acetonitrile for 15 min at ambient temperature. The guanidine phosphoramidate linkages were also obtained via Staudinger reaction, from 0.5 M solution of 2-azido-1,3-dimethylimidazolinium hexafluorophosphate (aber GmbH) in dry acetonitrile for 15 min at ambient temperature. Amidites were dissolved to 0.1 M in acetonitrile and incorporated using 3 min. coupling time for DNA amidites and 6 min. coupling time for all other amidites. After synthesis, oligonucleotides were cleaved from CPG and deprotected at room temperature in 28%-30% ammonium hydroxide and/or 50%/50% mixture of 28%-30% ammonium hydroxide/40% aqueous methylamine (AMA) for 36 hours or 2 h, respectively. Deprotected oligonucleotides were directly adsorbed on GlenPak cartridges and purified DMT-ON. Purified oligonucleotides were dried down, desalted, quantified by means of UV-Vis spectrophotometry and reconstituted in 1×PBS for use in biological experiments. Compound identity was confirmed by LC-MS (Column: DNA-Pac RP; Total flow: 0.5 mL/min.; Oven temperature: 50° C.; Total run time: 10 min.; Eluent gradient: 15-60% B in A; Mobile Phase A: 8 mM Triethylamine (TEA) and 200 mM HFIP in LC-MS grade water; Mobile Phase B: LC-MS grade MeOH).

GalNac: For triantennary GalNAc coupled oligonucleotides, GalNAc phosphoramidite (e.g., Hongene, cat. no. OP-042) was used for 5′ functionalization of oligonucleotides in an automated fashion. After synthesis and deprotection, oligonucleotides were purified by means of RP-HPLC (Column: Hypersil Gold Semiprep.; Total flow: 3 mL/min.; Oven temperature: 50° C.; Total run time: 40 min.; Eluent gradient: 0-100% B in A; Mobile Phase A: 100 mM phenylboronic acid (PBA) in [10% MeOH/90% 0.2 M aqueous NaOAc]; Mobile Phase B: 100 mM phenylboronic acid (PBA) in [90% MeOH/10% 0.2 M aqueous NaOAc]) and desalted by precipitation with excess of EtOH. After purification quality control and formulation was performed as for oligonucleotides without GalNAc.

NGS amplicon sequencing: Amplicon sequencing was used to analyse genetic variations of the target regions. To avoid biases in reverse transcription (RT) mRNA was heated to 90° C. for 2 min with an excess of a sense primer prior to RT. For target amplification of the editing region, a reverse transcription and cDNA amplification was performed with Luna Universal One-Step RT-qPCR mix (NEB) in a 10 μl reaction in a 384-well plate. Both the forward and reverse primer had an overhang to enable a second PCR with primers that bind to that overhang. As presented in Table 1, the following primers were used:

TABLE 1
SERPINA1 E342K Primer Sequences.

| | | | SEQ |
|---|---|---|---|
| | | | ID |
| Target | Primer | Sequence | NO. |
| SERPINA1 | Forward | TCGTCGGCAGCGTCAGATGTGTAT | 15 |
| E342K | primer | AAGAGACAGCACCCCTGAAGCTCT | |
| | | CCAAG | |
| SERPINA1 | Reverse | GTCTCGTGGGCTCGGAGATGTGTA | 16 |
| E342K | primer | TAAGAGACAGGGATAGACATGGGT | |
| | | ATGGCCTC | |
| SERPINA1 | Sense | GCATAAGGCTGTGCTGACCATCGA | 17 |
| E342K | Primer | CCCGAAAGGGACTGAAGCTGCTGG | |
| | | GGCCATGAA | |
| SERPINA1 s1 | Forward | TCGTCGGCAGCGTCAGATGTGTAT | 18 |
| | primer | AAGAGACAGGCATCACTAAGGTCT | |
| | | TCAGCAATG | |
| SERPINA1 s1 | Reverse | GTCTCGTGGGCTCGGAGATGTGTA | 19 |
| | primer | TAAGAGACAGGGATAGACATGGGT | |
| | | ATGGCCTC | |
| SERPINA1 s1 | Sense | AGCTCTCCCCGGCCGTGCATAAGG | 20 |
| | Primer | CTGTGCTGACATA | |

Subsequently, a second PCR was performed on the PCR product of the first PCR using OneTaq Hot-Start 2×MM with GC buffer (NEB) and forward and reverse primers containing unique indexes as well as adapters for Illumina sequencing. Afterwards, the samples were pooled, and the DNA library was purified with the NucleoSpin Gel and PCR Clean-up Kit (Macherey-Nagel), diluted and sequenced together with a PhiX library on an iSeq 100 (Illumina). Results were analysed using a Python script. Briefly, demultiplexed reads were filtered by quality, length and position before editing percentages were calculated by dividing the number of G reads by the sum of the number of G reads and A reads at the respective target site. Data are represented as mean percentage of editing±standard deviation (SD). Replicates are indicated by individual data points in the graphs.

### Example 1. Oligonucleotide AI-2863 Demonstrates Improved A-to-I Editing of the SERPINA1 Target and Higher M-AAT Levels In Vivo

Chemical modifications play an important role in improving pharmacokinetics of therapeutically relevant oligonucleotides, as they serve to, for example, enhance target binding, oligonucleotide stability, cell-specific targeting, and mitigate both sequence-specific as well as immune-related off-target effects. Also, M-AAT protein refers to the normal (wildtype) variant of the alpha-1 antitrypsin (AAT) protein, encoded by the SERPINA1 gene, which plays a crucial role in protecting tissues, particularly in the lungs, from enzyme damage caused by neutrophil elastase. Hence, experiments were conducted to test specific chemically modified oligonucleotides for their A-to-I RNA editing properties and their ability to induce M-AAT levels in vivo.

In vivo studies: In vivo studies were performed using the NSG-PiZ murine model mouse model. Specifically, studies were performed in mice by Synovo GmbH (Tübingen, Germany) in accordance with procedures approved by the Regional Council (Regierungspräsidium Tübingen, BW, Germany). Mice were housed on a 12:12 light-dark cycle, with ad libitum access to food and water. Mice expressing the human SERPINA 15342K transgene in C57BL/6J background were used. Mice homozygous for the human transgene (PiZ) were crossbred with wild-type C57BL/6J and offspring was used in all experiments. Eight-to-ten-week-old male and female mice were subcutaneously injected with 2.5 mg/kg, 5 mg/kg, or 10 mg/kg of the respective ASO. ASO dissolved in PBS or PBS only as indicated. Injections were performed on experimental day 0. Animals were sacrificed 7 days after the first dose and livers were collected and snap-frozen. Tissues were lysed in buffer RLT (RNeasy mini kit, Qiagen) with a bead homogenizer (Bead Mill Max, VWR) and 1.4 mm ceramic beads, at 4.5 m/s for 30 sec. The lysates were used for total RNA purification using RNeasy mini kit (Qiagen) with an on-column DNase I digest. The obtained RNA was then processed as described above to perform NGS amplicon sequencing and determining the RNA editing yield.

LC-MS levels: NSG-PiZ mouse serum samples were analysed by LC-MS/MS according to a protocol from Mayo Clinic with minor modifications (Chen et al., Clin. Chem., 2011; DOI: 10.1373/clinchem.2011.163006). Calibration standards and QC samples were prepared from alpha-1 antitrypsin (A1AT) isolated from human plasma (Athens Research & Technology, CAT #16160116091 MG) in surrogate matrix of 0.2% BSA in water. Calibration standard, QC, and serum sample dilutions (5 μL) were denatured with trifluoroethanol (TFE; 25 μL), diluted with 25 μL of 100 mM ammonium bicarbonate, and reduced with 10 μL of 50 mM dithiothreitol (DTT). After shaking at 55° C. for 30 minutes, the samples were treated with 10 μL of 150 mM iodoacetamide (IAA) and incubated for 30 min in the dark with shaking. The volumes were reduced to ˜25 μL by removing TFE under nitrogen for 10-15 min at 60° C. The samples were diluted with 45 μL of 100 mM ammonium bicarbonate and treated with 10 μL of 0.1 mg/mL trypsin at 37° C. overnight with shaking. The digestion was terminated by adding 50 μL of 2% formic acid solution, with simultaneous addition of isotopically labelled peptides: 10 nM of AVLTIDEK peptide (for wild-type A1AT quantification at the E342K, or ‘Z’, site) and 40 nM of prototypic SASLHLPK peptide (for total A1AT quantification). The samples were loaded on a Water Acquity Premier HSS T3 column (1.8 μm, 2.1×100 mm) and separated on a Water Acquity UPLC system with a gradient of 0.1% fluoric acid in water (mobile phase A) and acetonitrile (mobile phase B). Analytes were detected on a Sciex Triple Quad 5000 mass spectrometer, using positive ion electrospray with multiple reaction monitoring. Peaks corresponding to wild-type and total A1AT were identified by comparing the retention times to the respective labelled peptides. A calibration curve was constructed by calculating the area ratios for the unlabelled and labelled prototypic and wildtype Z peptide at each concentration of the A1AT standard. Wild-type and total A1AT levels in the experimental samples were determined by interpolation to the standard curve.

M-AAT serum levels: M-AAT serum levels were quantified by enzyme-linked immunosorbent assay (ELISA). Total and wild-type (M) AAT protein were detected by ELISA. For the quantification of total AAT, MaxiSorp 96-well plates (Thermo Fisher, 442404) were coated with a rabbit anti-human a1AT antibody (Dako Corporation, A0012). The following day, plates were washed and incubated with a blocking solution. After blocking, diluted standards (Athens Research and Technology, 16-16-011609) and samples were added to the plate. A second antibody, goat anti-human a1AT-HRP (Bethyl, A80-122P), was added to the plate. After reaction with OPD peroxidase substrate (Sigma Aldrich, P6912-50TAB) reactions were stopped by adding 2.5 M H2SO4 (Sigma-Aldrich, 258105-100ML) and plates were read at 490 nm on a SpectraMax i3x microplate reader (Molecular Devices). For the quantification of M-AAT, MaxiSorp 96-well plates (Thermo Fisher, 442404) were coated with a mouse anti-human M-AAT monoclonal antibody (Brantly Lab, University of Florida, HL1314). The following day, diluted standards (Athens Research and Technology, 16-16-011609) and samples were added to the plate. A second antibody, goat anti-human a1AT-HRP (Bethyl, A80-122P) was added to the plate. After reaction with OPD peroxidase substrate (Sigma Aldrich, P6912-50TAB) reactions were stopped by adding 2.5 M H2SO4 (Sigma-Aldrich, 258105-100ML) and plates were read at 490 nm on a SpectraMax i3x microplate reader (Molecular Devices). Concentrations of total and M-AAT were determined by interpolation of absorbance values on a standard curve.

Statistical Analysis: One-way ANOVA relative to Day-7 vehicle with Bonferroni multiple-comparisons correction. *p<0.05; **0.001≤p<0.01; ***0.0001≤p<0.001; ****p<0.0001.

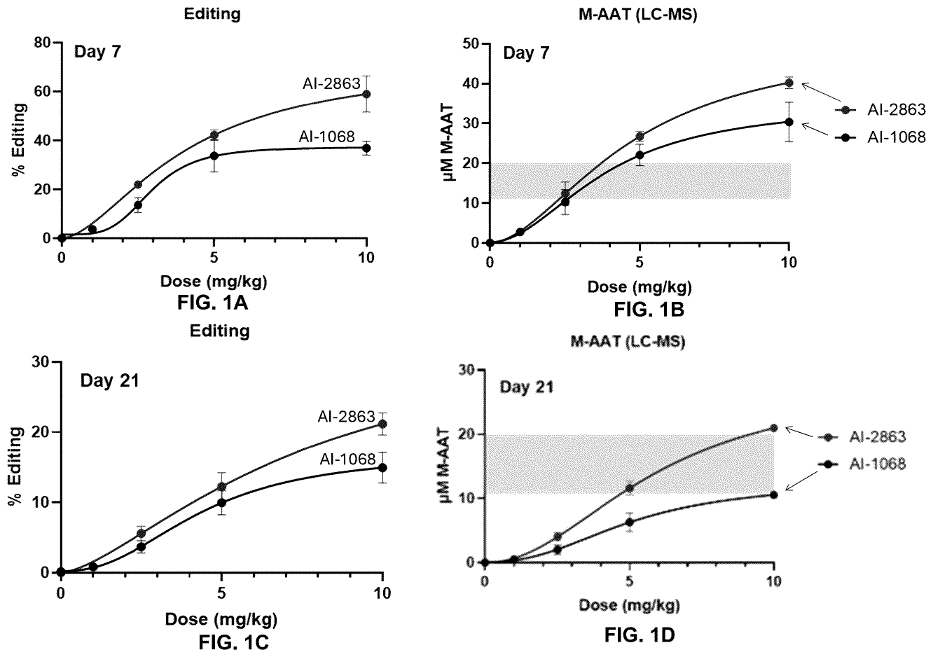

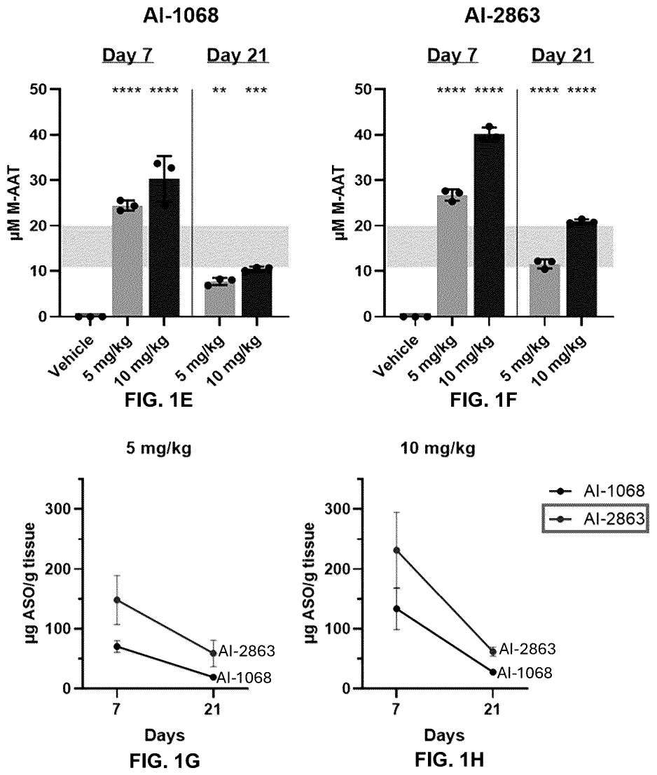

The oligonucleotide constructs, their respective modification pattern and sequence are listed in Table 2. The results are shown in FIGS. 1A-1H. Vehicle what was used as control (20 mM Tris HCl, pH 7.4+8% w/v sucrose).

TABLE 2
Oligonucleotide constructs and modifications.

| Construct | | Seq. | FIG. |
|---|---|---|---|
| (asymmetry) | Sequence (5′ to 3′ direction) | ID | No. |
| AI-1068 | Mesyl: “+24, +21, +13, +4, −2, −8”; PO | 4 | 1 |
| (25-1-8) | 22, 17, 16, 11, 8, 5, 3, 2, −3 | | |
| | mG&mC*mCfC&fC*mA*fG*mCmoeAfG*fC*moeT&fU*mCf | | |
| | A*mG*fUmoe(MeC)*fC*moe(MeC)fU&mUmoeTfC*moeT* | | |
| | dC*dI&mUfC*moeG*mA*mU*mG&mG(Ser)(GalNac) | | |
| AI-2863 | Mesyl: “+24, +13, −2, −8”; PO 23, 17, 16, | 7 | 1 |
| (25-1-8) | 12, 8, 5, 3,2, −3, −5 | | |
| | mG&mCmC*fC*fC*mA*fG*mCmoeAfG*fC*moeT&mUmC* | | |
| | fA*mG*mUmoe(MeC)*fC*moe(MeC)fU*mUmoeTfC*moe | | |
| | T*dC*dI&mUfC*moeGmA*mU*mG&mG*(C7)(GalNac) | | |

In Table 2:
d = 2′-H (deoxyribose; DNA); PO = phosphate; * = phosphorothioate (PS); & = (mesyl) methanesulfonyl;
m = 2′-OMe; f = 2′-fluoro; moe = 2′-O-methoxyethyl (2′-MOE); I = inosine; MeC = 5′-methylcytidine;
d = 2′-H (deoxyribose; DNA); PO = phosphate; * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine.

GalNac=N-acetylgalactosamine represented by the structure below:

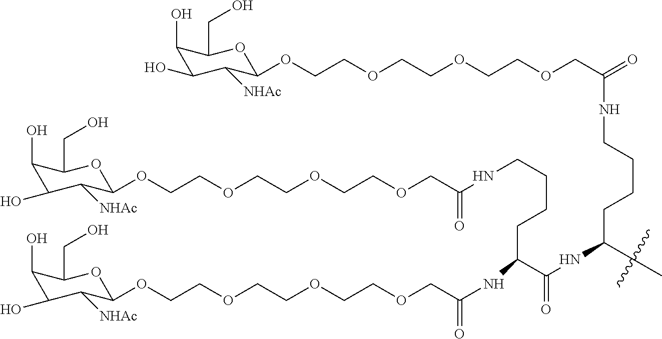 [SMILES: *[C@H](CCCCNC(=O)COCCOCCOCCOC1OC(CO)C(O)C(O)C1NC(C)=O)NC(=O)[C@H](CCCCNC(=O)COCCOCCOCCOC1OC(CO)C(O)C(O)C1NC(C)=O)NC(=O)COCCOCCOCCOC1OC(CO)C(O)C(O)C1NC(C)=O]

(C7)=C7 linker represented by the structure below, wherein attachment point A links to the GalNac and attachment point B links to the 3′-terminal nucleoside:

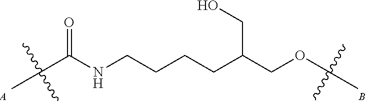

(Ser)=serinol linker represented by the structure below, wherein attachment point A links to the GalNac and attachment point B links to the 3′-terminal nucleoside:

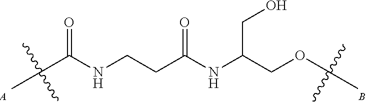

“*(C7) (GalNac)” in AI-2863 is represented by the structure below:

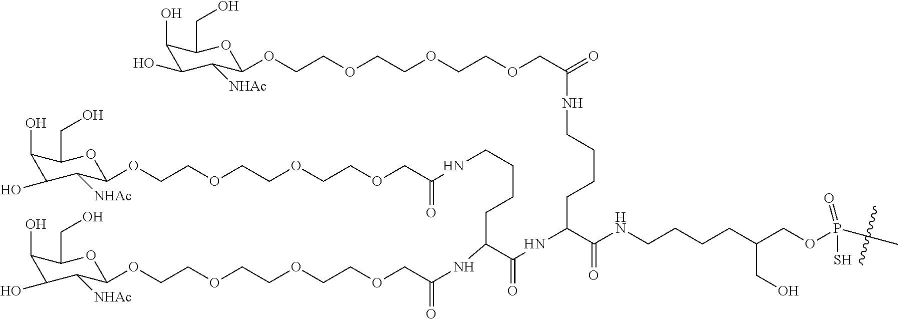 [SMILES: *P(=O)(S)OCC(CO)CCCCNC(=O)C(CCCCNC(=O)COCCOCCOCCOC1OC(CO)C(O)C(O)C1NC(C)=O)NC(=O)C(CCCCNC(=O)COCCOCCOCCOC1OC(CO)C(O)C(O)C1NC(C)=O)NC(=O)COCCOCCOCCOC1OC(CO)C(O)C(O)C1NC(C)=O]

As shown in FIG. 1A, AI-1068 and AI-2863 showed efficient target editing by day 7. However, AI-2863 demonstrated significantly improved target editing (%) when compared to AI-1068. To determine whether this increase in target editing correlated with an improvement at M-AAT protein levels, M-AAT protein levels were determined by LC-MS. Notably, the normal plasma concentration of AAT ranges from 80 mg/dL to 220 mg/dL (20 to 48 μmol/L) using nephelometry or 150 mg/dL to 350 mg/dL by radial immunodiffusion. The AAT clinical threshold is 11 μmole/L. As shown in FIG. 1B, for both AI-2863 and AI-1068, plasma M-AAT levels reached and surpassed the clinical threshold levels by day 7. Remarkably, administration of AI-2863 led to higher levels in plasma concentrations of M-AAT when compared to AI-1068, confirming that an increase in target editing correlates to an increase in M-AAT protein levels.

While there was a general decrease in the percentage target editing and M-AAT levels by day 21, a similar trend was observed. That is, by day 21, AI-2863 demonstrated higher levels of target editing than AI-1068 at 5 mg/kg as well as 10 mg/kg (FIG. 1C). This was also reflected in persistently higher M-AAT levels. At a dose of 5 mg/kg, AI-2863 reached the lower end of the therapeutic threshold and surpassed it at a dose of 10 mg/kg by day 21 (FIG. 1D). On the other hand, at a dose of 10 mg/kg, AI-1068 only reached the lower end of the therapeutic threshold by day 21 (FIG. 1D).

As summarised in FIGS. 1E-1F, the data demonstrate that oligonucleotide AI-2863 demonstrates significantly better target editing and higher M-AAT levels by day 7 and day 21 in the NSG-PiZ mouse models at multiple dose levels when compared to AI-1068.

The overall statistical results for target editing and M-AAT levels of AI-2863 relative to AI-1068 are presented in Table 3.

TABLE 3
AI-2863 target editing and M-AAT levels relative to AI-1068.

| Construct | Day | 2.5 mg/kg | 5 mg/kg | 10 mg/kg |
|---|---|---|---|---|
| AI-2863 | 7 | * | ns | **** |
| (target editing) | | (higher) | | (higher) |
| AI-2863 | 7 | ns | * | ** |
| (M-AAT level) | | | (higher) | (higher) |
| AI-2863 | 21 | ns | ns | * |
| (target editing) | | | | (higher) |
| AI-2863 | 21 | * | **** | **** |
| (M-AAT level) | | (higher) | (higher) | (higher) |

To further evaluate tissue accumulation of each specific oligonucleotide, the levels of AI-2863 and AI-1068 were analysed. It was shown that there was significant increase in oligonucleotide tissue exposure in case of AI-2863 when compared to AI-1068 at both 5 mg/kg and 10 mg/kg (FIGS. 1G-1H). Although there was a general decrease in oligonucleotide concentration by day 21, AI-2863 consistently showed higher tissue levels. The statistical results relative to AI-1068 are presented in Table 4.

TABLE 4
Significant changes in tissue exposure relative to AI-1068.

| | |
|---|---|
| Day 7 | Day 21 |

| Construct | 5 mg/kg | 10 mg/kg | 5 mg/kg | 10 mg/kg |
|---|---|---|---|---|
| AI-2863 | ** | ns | *** | ** |

Overall, the results demonstrate that AI-2863 has superior SERPINA1 target editing properties than AI-1068 (FIGS. 1A and 1C). This improvement in target editing by AI-2863 is further reflected in higher M-AAT expression levels (FIGS. 1B and 1D) and tissue exposure (FIGS. 1G-1H).

Notably, AI-2863 is identical in sequence to AI-1068. However, it differs in its chemical modification pattern. In the AI-2863 construct, two 2′F were replaced by 2′Ome modifications (at positions +13 and +9), two mesyl linkages were replaced by PS linkages (at positions +21 and +4) and three PS linkage were changed to PO (positions +23, +12, and −5). Also, a PS linkage at position +11 in AI-1068 was changed to a PS linkage at position +11 in AI-2863. In addition, in the AI-2863 construct, the GalNac residue is linked to the terminal nucleotide at the 3′ end via a C7 linker (see, FIG. 8). Hence, the inventors identified four key instability sites around positions +22, position +11, position +9, and position −8 (linkage between the final nucleotide and GalNac residue).

Overall, the results confirm that modifications in the oligonucleotide pattern enhance target editing and M-AAT protein levels. Systematic stabilization of specific positions with PS, 2′MOE, and 2′F modifications improved the potency and durability of the oligonucleotide, regardless of its sequence identity.

### Example 2. Oligonucleotide AI-2863 Demonstrates Improved Potency and Durability in the Liver of B6 AATD Mice In Vivo

To further evaluate the in vivo potency of the AI-2863, B6 AATD mice were administered the oligonucleotide and assessed for its efficacy to mediate target editing in the liver. To do so, a first liver study was performed, followed by a subsequent repeat study.

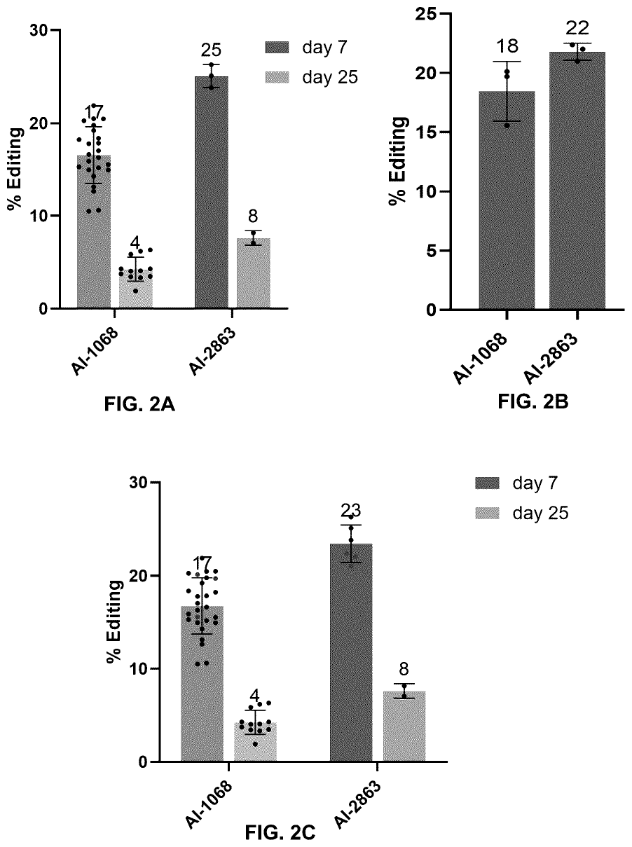

In vivo studies: Studies were conducted in the B6 PiZ murine model as described for Example 1. Samples were analysed on day 1 and day 7. The oligonucleotide constructs, their respective modification pattern and sequence are listed in Table 5. The results are shown in FIGS. 2A-2C.

TABLE 5
Oligonucleotide constructs and modifications.

| Construct | | Seq. | FIG. |
|---|---|---|---|
| (asymmetry) | Sequence (5′ to 3′ direction) | ID | No. |
| AI-1068 | mG&mC*mCfC&fC*mA*fG*mCmoeAfG*fC*moeT&fU*mCf | 4 | 2 |
| (25-1-8) | A*mG*fUmoe(MeC) *fC*moe(MeC)fU&mUmoeTfC*moeT* | | |
| | dC*dI&mUfC*moeG*mA*mU*mG&mG(Ser)(GalNac) | | |
| AI-2863 | mG&mCmC*fC*fC*mA*fG*mCmoeAfG*fC*moeT&mUmC*f | 7 | 2 |
| (25-1-8) | A*mG*mUmoe(MeC)*fC*moe(MeC)fU*mUmoeTfC*moeT* | | |
| | dC*dI&mUfC*moeGmA*mU*mG&mG*(C7)(GalNac) | | |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine; GalNac = N-acetylgalactosamine; (C7) = C7 linker; (Ser) = serinol linker.

The data demonstrate that AI-2863 mediates improved target editing by day 7 and by day 25 when compared to AI-1068 (FIG. 2A). While AI-1068 reached about 17% target editing by day 7, AI-2863 reached about 25% by day 7. In a repeat study, AI-2863 still better target editing in the liver than AI-1068 (FIG. 2B). As summarized in FIG. 2C, combining the results showed a significant improvement in target editing in the B6 AATD mouse model when administering AI-2863 compared to AI-1068.

Overall, these results further confirm that changes in the oligonucleotide modification pattern lead to enhanced target editing.

### Example 3. Oligonucleotide AI-2863 Shows No Detectable Organ Toxicity and Remains Safe at High Doses In Vivo

Alanine aminotransferase (ALT) and aspartate aminotransferase (AST) are enzymes generally found within liver at high concentrations. Hence, ALT and AST are commonly used as a biomarkers for hepatic injury. At the same time, creatinine and BUN (blood urea nitrogen) are both used to assess kidney function. Further, the immunogenicity of each oligonucleotide (AI-2863 and AI-1550) was determined. Notably, the AI-1550 construct corresponds to AI-1068 containing a C7 linker instead of a serinol linker between the terminal nucleotide at the 3′ end of the oligonucleotide and the GalNac residue.

Measurement of clinical chemistries: Mouse blood was collected either by cardiac puncture (terminal bleeding) or by tail punction (survival bleeding). Blood was collected into Serum-Gel CAT tubes and kept at room temperature for at least 30 minutes, and then centrifuged at 10.000 g for 5 minutes to generate serum. Alanine aminotransferase (ALT) and aspartate aminotransferase (AST), and the biomarkers of kidney function (Creatinine and Urea) were measured on Respons910 automatic analyzer (DiaSys Diagnostic Systems GmbH). Blood urea nitrogen (BUN) was calculated from the total urea (mg/dL) multiplying it by 0.467. PBS served as negative control.

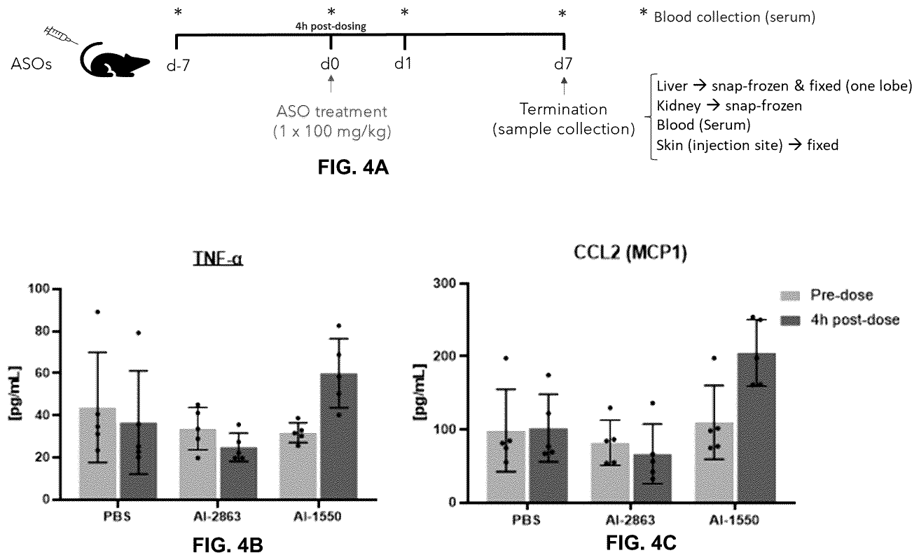

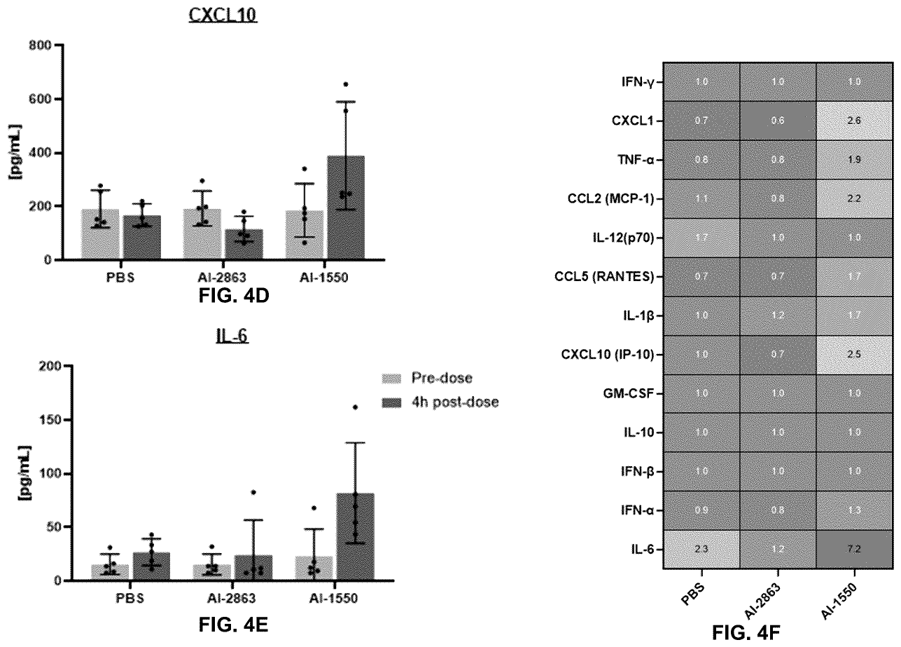

Cytokine profile: B6-PiZ mice were administered 100 mg/kg of the respective oligonucleotide to determine the immunogenicity of each oligonucleotide. Blood samples were taken 7 days prior to administration (d-7), 4 hrs post dosing (day 0), on day 1 and day 7 post administration (FIG. 4A). Blood and tissue samples were subsequently analysed. The level of immunogenetic markers TFN-α, CCL2 (MCP1), CXCL10 and IL-6 was determined in samples taken pre-dose and 4 hours after dosing. PBS served as control.

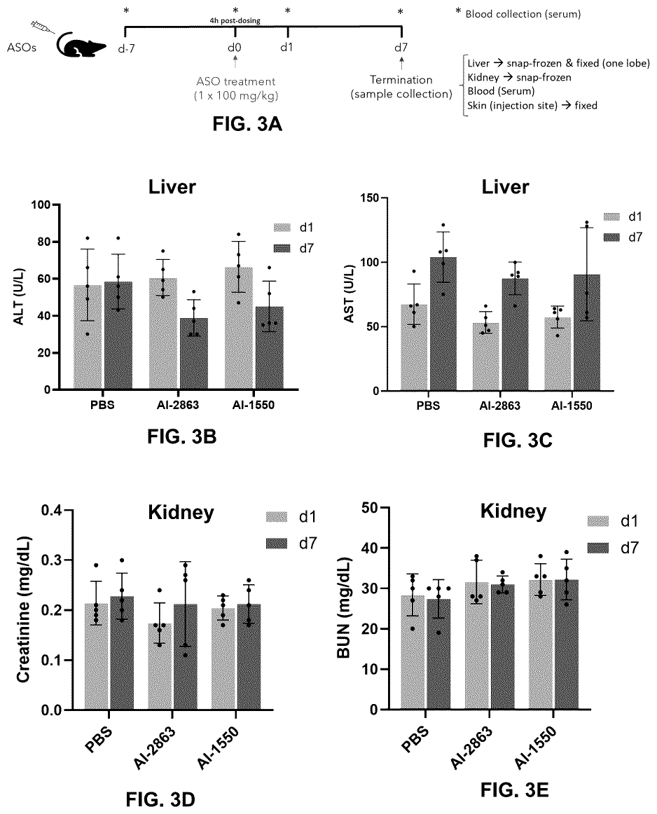

The oligonucleotide constructs, their respective modification pattern and sequence are listed in Table 6. The results are shown in FIGS. 3A-3E (cytotoxicity) and FIGS. 4A-4F (immunogenicity).

TABLE 6
Oligonucleotide constructs and modifications.

| Construct | | Seq. | FIG. |
|---|---|---|---|
| (asymmetry) | Sequence (5′ to 3′ direction) | ID | No. |
| AI-2863 | mG&mCmC*fC*fC*mA*fG*mCmoeAfG*fC*moeT&mUmC* | 7 | 3, 4 |
| (25-1-8) | fA*mG*mUmoe(MeC)*fC*moe(MeC)fU*mUmoeTfC*mo | | |
| | eT*dC*dI&mUfC*moeGmA*mU*mG&mG*(C7)(GalNac) | | |
| AI-1550 | mG&mC*mCfC&fC*mA*fG*mCmoeAfG*fC*moeT&fU*m | 12 | 3, 4 |
| (25-1-8) | CfA*mG*fUmoe(MeC)*fC*moe(MeC)fU&mUmoeTfC*mo | | |
| | eT*dC*dI&mUfC*moeG*mA*mU*mG&mG(C7)(GalNac) | | |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine; GalNac = N-acetylgalactosamine; (C7) = C7 linker.

Studies were conducted as outlined in FIG. 3A. Samples were collected at various time points as assessed 1 day and 7 days post administration. As shown in FIGS. 3A-3E, AI-2863 showed no significant toxicity by day 1 and day 7, and remained safe at high doses in B6 AATD mice. As shown in FIGS. 3B-3C, no significant change in their levels could be observed, suggesting that there was no impact on the level of liver biomarkers or induction of hepatic injury. Similarly, there was no significant change in kidney function biomarkers, as shown by stable expression levels of creatine and blood urea nitrogen (BUN) (FIGS. 3D-3E).

As shown in FIGS. 4A-4F, AI-2863 was demonstrated to be safe in B6 mice even at high dose. Notably, in the presence of high doses of the oligonucleotide (100 mg/kg), no significant increase in immunogenic markers could be detected (FIGS. 4B-4E). Specifically, while there was no noticeable increase in TNF-α expression for AI-2863 4 h post-dosing, there was a TNF-α increase in animals subjected to the AI-1550 construct (FIG. 4B). Similarly, for animals in the AI-1550 group, an increase in CCL2, CXCL10, and IL-6 could be detected 4 h post administration (FIGS. 4C-4E). Overall, for AI-2863, all cytokines showed a <2-fold increase 4 h post treatment (FIG. 4F). On the other hand, there was a mild increase of some cytokines for AI-1550, and in particular for IL-6. These results demonstrate that candidate AI-2863 was well tolerated in vivo and better tolerated than AI-1550 at a high dose of 100 mg/kg.

Overall, these data show that AI-2863 has superior toxicity and immunogenicity profiles compared to AI-1550 and further demonstrate that AI-2863 has no significant in vivo toxicity or immunogenicity at high dosage.

### Example 4. Oligonucleotide AI-2863 Shows No Detectable Signs of Cytotoxicity in In Vitro Safety Assays

Cell lysis or cell death is associated with the release of cytoplasmic contents including, for example, ATP, the energy source of living cells, and the enzyme lactate dehydrogenase (LDH). On the other hand, caspases and caspase activation play a central role in cell death and inflammation responses. To further assess the safety profile of the lead oligonucleotide cytotoxicity was assessed in mouse fibroblast cells (NIH-3T3 cells) in vitro by determining the extracellular ATP content, LDH release, and caspase activation.

Transfection: NIH-3T3 fibroblast cells were transfected with either 4 nM, 20 nM, or 100 nM of the respective oligonucleotide. 48 h hours after transfection, cells were assessed. Transfection agent without ASO (UTC) and AI-0139-001 served as negative control. AI-0137-003 (Inclisiran Fluorochem) served as positive control. AI-0137-003 is a small interfering ribonucleic acid (siRNA) molecule directed at PCSK9 to lower circulating LDL-C, which has been approved by the US FDA and EMA.

ATP content assay: Oligonucleotides were transfected into embryonic mouse fibroblast cell line NIH3T3 24 h after seeding in a 96-well well plate (3,750 cells/well). For quantitative analysis of intracellular ATP content, CellTiter-Glo 2.0 Cell Viability Reagent (Promega) was added directly to cells at a volume equal to the sample volume 24 and 48 h post oligonucleotide transfection. Luminescence was recorded after 1 hour incubation using SpectraMax i3x (molecular Devices) plate reader. Background readings determined from wells containing culture medium only were subtracted. Relative ATP content was calculated as 100%×luminescence reading of a treated sample/a untreated control.

LDH assay: Cell damage was monitored by Lactate dehydrogenase (LDH) release. LDH is a soluble cytosolic enzyme present in many cell types that is rapidly released into the cell culture medium upon disruption of the plasma membrane. ASOs were transfected into embryonic mouse fibroblast cell line NIH3T3 24 h after seeding in a 96-well well plate (3,750 cells/well). For quantitative analysis of lactate dehydrogenase (LDH) released into the culture media, culture media was diluted 100× in LDH storage buffer (200 mM Tris-HCl, 10% Glycerol, 1% BSA in DPBS) and mixed to equal volume of LDH-Glo Cytotoxicity Detection Reagent (Promega). Luminescence was recorded after 1 hour incubation using SpectraMax i3x (molecular Devices) plate reader. Background readings determined from wells containing culture medium only were subtracted. Relative LDH content was calculated as 100%× luminescence reading of a treated sample/a untreated control.

Caspase 3/7 activation assay: Oligonucleotides were transfected into embryonic mouse fibroblast cell line NIH3T3 24 h after seeding in a 96-well well plate (3,750 cells/well). For quantitative analysis of caspase 3/7 activation, Caspase-Glo 3/7 Reagent (Promega) was added directly to cells at a volume equal to the sample volume 24 and 48 h post ASO transfection. Luminescence was recorded after 1 hour incubation using SpectraMax i3x (molecular Devices) plate reader. Background readings determined from wells containing culture medium only were subtracted. Relative caspase activity was calculated as 100%×luminescence reading of a treated sample/a untreated control.

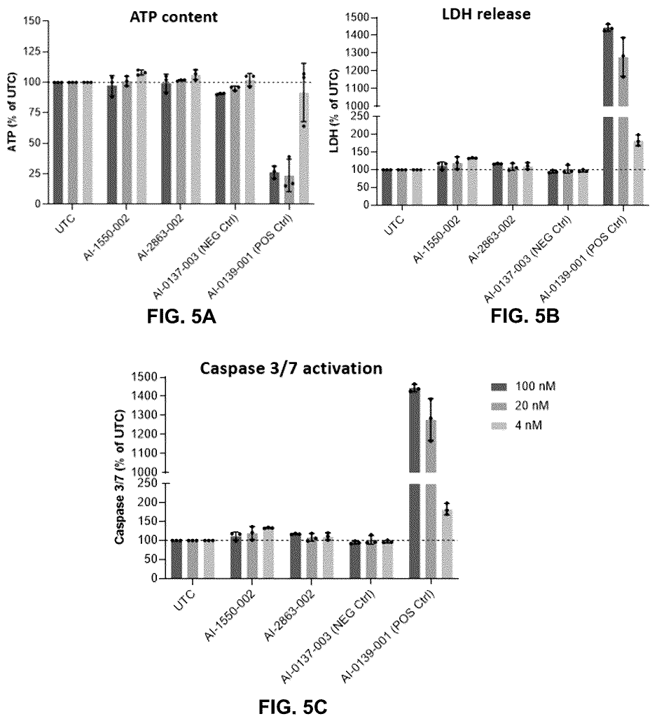

The oligonucleotide constructs, their respective modification pattern and sequence are listed in Table 7. Untransfected cells (UTC) were used as negative control. The results of the toxicity studies are shown in FIGS. 5A-5C.

TABLE 7
Oligonucleotide constructs and modifications.

| Construct | | Seq. | FIG. |
|---|---|---|---|
| (asymmetry) | Sequence (5′ to 3′ direction) | ID | No. |
| AI-2863 | mG&mCmC*fC*fC*mA*fG*mCmoeAfG*fC*moeT&mUmC*f | 7 | 5 |
| (25-1-8) | A*mG*mUmoe(MeC)*fC*moe(MeC)fU*mUmoeTfC*moeT* | | |
| | dC*dI&mUfC*moeGmA*mU*mG&mG*(C7)(GalNac) | | |
| AI-1550 | mG&mC*mCfC&fC*mA*fG*mCmoeAfG*fC*moeT&fU*mCf | 12 | 5 |
| (25-1-8) | A*mG*fUmoe(MeC)*fC*moe(MeC)fU&mUmoeTfC*moeT*d | | |
| | C*dI&mUfC*moeG*mA*mU*mG&mG(C7)(GalNac) | | |
| AI-0139 | InG*In(MeC)*In(MeC)*dT*dC*dC*dC*dA*dG*dT*dT* | 13 | 5 |
| | dC*dC*InT*InT*InT | | |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine; In = locked nucleic acid; GalNac = N-acetylgalactosamine; (C7) = C7 linker.

As demonstrated in FIG. 5A, no significant decrease in ATP content could be observed in the presence of AI-2863 or AI-1550 when compared to UTC control and negative control (AI-0137-003; Inclisiran Fluorochem). However, in the presence of AI-0139-001 (positive control) there was a decrease in ATP content to about 25% at 20 nM and 100 nM. Similarly, as detected by the LDH release assay, AI-1550 and AI-2863 did not induce any significant cytotoxic effects even at 100 nM (FIG. 5B). Similarly, no significant caspase activation could be observed for either of the two oligonucleotides when compared to control (FIG. 5C).

These results show that neither AI-1550 nor AI-2863 induce a significant cytotoxic effect in vitro.

### Example 5. Oligonucleotide AI-2863 Shows No Significant In Vitro Immune Activation in Human Peripheral Blood Mononuclear Cells (PBMCs)

Administration of oligonucleotides has been associated with the activation of the innate immune system through interactions with toll-like receptors (TLRs) (Levin, 2019). To investigate the potential or absence thereof of the oligonucleotides to induce an inflammatory response, the levels of inflammatory marker genes TNF-α (tumor necrosis factor alpha) and IL-6 (interleukin-6) determined.

Immunotoxicity: The immunotoxic potential of the oligonucleotides was evaluated by assessing the changes in pro-inflammatory cytokine serum levels (TNFα, and IL-6) in hPBMCs. Briefly, oligonucleotides were delivered into human PBMC (Bioivt) immediately after seeding cells in a 96-well U-bottom plate (500,000 cells/well). Determination of cytokine levels in cell supernatants 24 h after ASO gymnosis was performed by ELISA using LEGENDplex Human Anti-Virus Response Panel 1 (BioLegend) according to the manufacturer's protocol. Cytokine levels (pg/mL) were normalized to untreated cells.

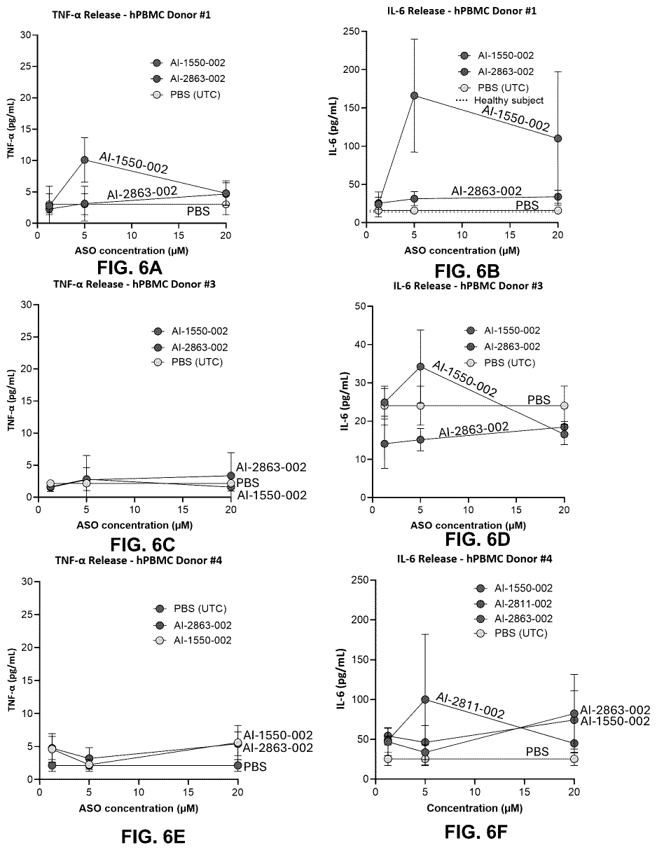

The oligonucleotide constructs, their respective modification pattern and sequence are listed in Table 8. Untransfected cells (UTC) were used as negative control. The results are shown in FIGS. 6A-6F.

TABLE 8
Oligonucleotide constructs and modifications.

| Construct | | Seq. | FIG. |
|---|---|---|---|
| (asymmetry) | Sequence (5′ to 3′ direction) | ID | No. |
| AI-1550 | mG&mC*mCfC&fC*mA*fG*mCmoeAfG*fC*moeT&fU*mCfA* | 12 | 6 |
| (25-1-8) | mG*fUmoe(MeC)*fC*moe(MeC)fU&mUmoeTfC*moeT*dC* | | |
| | dI&mUfC*moeG*mA*mU*mG&mG(C7)(GalNac) | | |
| AI-2863 | mG&mCmC*fC*fC*mA*fG*mCmoeAfG*fC*moeT&mUmC* | 7 | 6 |
| (25-1-8) | fA*mG*mUmoe(MeC)*fC*moe(MeC)fU*mUmoeTfC*moe | | |
| | T*dC*dI&mUfC*moeGmA*mU*mG&mG*(C7)(GalNac) | | |
| AI-2811 | mG&mCmC*fC*fC*mA*fG*mCmoeAfG*fC*moeT&fU*mC* | 5 | 6 |
| (25-1-8) | fA*mGmoeTmoe(MeC)fC*moe(MeC)fU*mUmoeTfC*moe | | |
| | T*dC*dI&mU*fC*moeGmAmU*mG&mG(C7)(GalNac) | | |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine; GalNac = N-acetylgalactosamine; (C7) = C7 linker.

Overall, no significant changes in cytokine production could be detected for AI-2863 for any of the donors suggesting that there was no significant immune activation. That is, no increase in TNF-α or IL-6 expression could be detected for any of the three donors. Only a slight increase in IL-6 was observed for AI-1550 in donor 1 (FIGS. 6A-6B). These data demonstrate that the oligonucleotides do not cause a dysregulation of the pro-inflammatory genes TNF-α and IL-6. The results further suggest that the oligonucleotides do not induce safety-relevant issues in major target organs.

### Example 6. Oligonucleotide AI-2863 Demonstrates Superior Target Editing and Persistent M-AAT in Head-to-Head Comparison

In Alpha-1 antitrypsin deficiency (AATD) the proper synthesis and secretion of alpha-1 antitrypsin (AAT) protein is impaired, leading to toxic hepatic accumulation along with pulmonary insufficiency. Hence, the effect of the AI-2863 lead oligonucleotide was not only determine at the molecular level but also at the M-AAT protein expression.

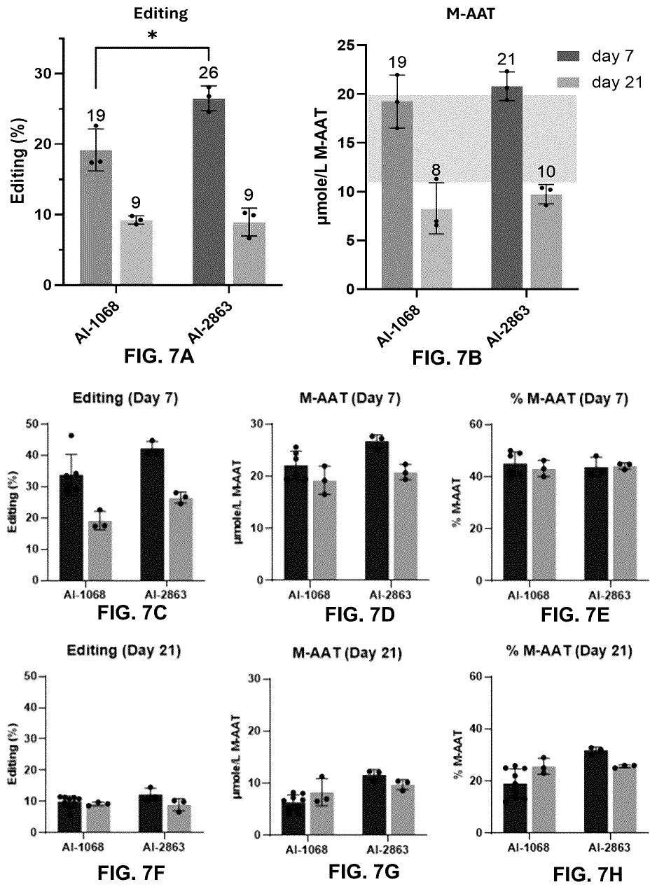

M-AAT serum levels were determined as generally described for Example 1. M-AAT protein levels were detected on day 7 and on day 21 post administration. The oligonucleotide constructs, their respective modification pattern and sequence are listed in Table 9. The results are shown in FIGS. 7A-7H.

TABLE 9
Oligonucleotide constructs and modifications.

| Construct | | Seq. | FIG. |
|---|---|---|---|
| (asymmetry) | Sequence (5′ to 3′ direction) | ID | No. |
| AI-1068 | mG&mC*mCfC&fC*mA*fG*mCmoeAfG*fC*moeT&fU*mCf | 4 | 7 |
| (25-1-8) | A*mG*fUmoe(MeC)*fC*moe(MeC)fU&mUmoeTfC*moe | | |
| | T*dC*dI&mUfC*moeG*mA*mU*mG&mG(Ser)(GalNac) | | |
| AI-2863 | mG&mCmC*fC*fC*mA*fG*mCmoeAfG*fC*moeT&mUmC*f | 7 | 7 |
| (25-1-8) | A*mG*mUmoe(MeC)*fC*moe(MeC)fU*mUmoeTfC*moe | | |
| | T*dC*dI&mUfC*moeGmA*mU*mG&mG*(C7)(GalNac) | | |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine; GalNac = N-acetylgalactosamine; (C7) = C7 linker; (Ser) = serinol linker.

As shown in FIG. 7A, AI-2863 demonstrated significantly higher target editing levels than AI-1068 by day 7. While there was a decrease in target editing by day 21, no significant difference was observed between A12863 and AI-1068. Similarly, AI-2863 showed a better overall M-AAT profile than AI-1068 (FIG. 7B). While both AI-1068 and AI-2863 reached M-AAT threshold levels by day 7, M-AAT levels were higher for AI-2863 by day 21 demonstrating better in vivo persistence (FIG. 7B). Further, cross comparing the data of different cohort studies showed consistent results in that AI-2863 led to a general improvement in target editing (FIGS. 7C-7D) and higher levels in M-AAT (FIG. 7F and FIG. 7H) when compared to AI-1068.

In summary, AI-2863 showed significantly better target editing efficacy (%) and induced higher M-AAT levels than AI-1550 (AI-1068 with C7 linker) in vitro (FIGS. 1A-1H and FIGS. 2A-2C). AI-2863 was well tolerated in high dose 100 mg/kg B6 mouse studies with no elevation in liver, kidney or inflammation biomarkers (FIGS. 3A-3E and FIGS. 4A-4F). AI-1550 showed some increase in inflammation biomarkers and some mice showed organ abnormalities. N₀ in vitro toxicity was monitored (FIGS. 5A-5C). AI-1550 (AI-1068) showed some cytokine elevation in one of three hPBMC donors, while no cytokine elevation was detected in AI-2863 across any of the three PBMC donors (FIGS. 6A-6F). The results of the various studies are summarised in Table 10.

TABLE 10
Summary of results from NSG-PiZ studies. Bold indicates that the minimum level was reached.

| | | | | | | | AI-1068 | |
|---|---|---|---|---|---|---|---|---|
| Parameter | | Model | Minimum | Desired | AI-2863 | | (AI-1550)** | |
| Efficacy | Dose | NSG-PiZ | 5-10 | 3-5 | 10 | 5 | 10 | 5 |
| | | murine model | mg/kg | mg/kg | mg/kg | mg/kg | mg/kg | mg/kg |
| | RNA | NSG-PiZ | 35% | >50% | 59% | 42% | 37% | 34% |
| | editing | murine model | | | | | | |
| | efficiency | (day 7) | | | | | | |
| | | NSG-PiZ | | | 21% | 12% | 15% | 10% |
| | | murine model | | | | | | |
| | | (day 21*) | | | | | | |
| | M-AAT | NSG-PiZ | >11 | >20 | 40 | 27 | 30 | 22 |
| | | murine model | μM | uM | μM | μM | μM | μM |
| | | (d 7) | | | | | | |
| | | NSG-PiZ | >6 | >11 | 21 | 12 | 11 | 6 |
| | | murine model | μM | μM | μM | μM | μM | μM |
| | | (d 21*) | | | | | | |
| | % M-AAT of | NSG-PiZ | >30% | >50% | 58% | 44% | 53% | 45% |
| | total | murine model | | | | | | |
| | | (d 7) | | | | | | |
| | | NSG-PiZ | >0 | >30% | 44% | 32% | 29% | 19% |
| | | murine model | | | | | | |
| | | (d 21*) | | | | | | |
| Safety | In vitro tox | NIH-3T3 | <3-fold ↑ | <2-fold ↑ | <2-fold ↑ | | <2-fold ↑ | |
| | (Caspase, | (100 nM) | | | | | | |
| | ATP, LDH) | | | | | | | |
| | Elevation | B6-PiZ | <5-fold ↑ | <3-fold ↑ | <3-fold ↑ | | <3-fold ↑ | |
| | in liver | murine model | | | | | | |
| | ALT/AST | (100 mg/kg) | | | | | | |

| Elevation in | B6-PiZ | <5-fold ↑ | <3-fold ↑ | <3-fold*** ↑ | IL-6 >5 fold |
|---|---|---|---|---|---|
| inflammation | murine model | | | | in PBMC and |
| (TNF-α/IL-6) | (100 mg/kg), | | | | B6*** |

human PBMC
(20 μM)

*Day 21 timepoint challenges the durability and is a good measure for dosing
**AI-1550 = AI-1068 with C7- instead of Serinol-linker

Overall, oligonucleotide AI-2863 had a better profile in several of the parameters assessed. This shows that the chemical modification pattern of the oligonucleotide can be improved to optimize potency and stability. The precise structure and modification pattern of AI-2863 is shown in FIG. 8.

### Example 7. Generation and Identification of New Oligonucleotides Containing Combinations of Chemical Modifications

Based on the efficacy and safety data obtained for AI-2863 and AI-1068 (AI-1550) (Table 10), AI-2863 was selected as the foundation for designing new compounds aimed at reducing oligonucleotide nuclease sensitivity and enhancing stability and potency. This effort led to the generation of several ASOs incorporating various combinations of chemical and sequence modifications. All ASOs featured a mesyl-modification pattern of “+24, +13, −2, −8” combined with additional sequence variations, internucleoside linkage (e.g., PS, PO, or mesyl (&) linkage) and 2′-sugar modifications. The different oligonucleotides were assessed for their transfection and target editing efficacy (%) as well as their overall impact on M-AAT serum levels.

Transfection in Hela Cells: Hela cells with genomically integrated SERPINA1 E342K were cultured in DMEM supplemented with 10% FBS (both Gibco) at 37° C. and 5% CO₂ and passaged every 3-4 days. Upon 80% confluence, cells were dissociated with Trypsin-ETDA (0.25%) and seeded at 7,500 cells/well in 96-well plates. After 24 hours, cells were transfected with ASOs at indicated final concentration using 0.3 μl Lipofectamine RNAiMAX (Invitrogen) per well in OptiMEM (Gibco). Transfection mix was prepared by mixing equal volumes of 10× concentrated ASO and transfection reagent, and 20 μl of transfection mix was transferred to cells containing 80 μl fresh culture medium. If not stated otherwise, cells were washed with PBS and harvested 24 hours after transfection in 125 μl/well lysis buffer (Dynabeads mRNA direct kit, Invitrogen). Lysates of 96-well plates were transferred to a 384-plate and mRNA was isolated using the Dynabeads mRNA direct kit and an automated plate washer (Cytena C. Wash).

Cell culture, free uptake and mRNA isolation in primary mouse hepatocytes: Freshly isolated primary mouse hepatocytes from transgenic hSERPINA1 E342K (PiZ) mice, were plated in 96-well collagen-coated plates (Greiner) at a density of 2.5×10⁴ cells per well (100 μL per well) in DMEM low glucose (Gibco) supplemented with 10% FBS (Gibco) and 1% penicillin/streptomycin (Gibco) for 4 hours, then they were cultured in William's E Medium (Gibco) supplemented with 1% GlutaMAX™ (Gibco) and 1% penicillin/streptomycin for 24 hours. The hepatocytes were cultured under standard culture conditions at 37° C., 5% CO₂ and in a humidified atmosphere. With the media change, cells were treated with different concentrations of ASOs in 1×PBS (Gibco) for the free uptake. Cells were washed with PBS and harvested 24 hours after ASO treatment in 125 μl/well lysis buffer (Dynabeads mRNA direct kit, Invitrogen). Lysates of 96-well plates were transferred to a 384-plate and mRNA was isolated using the Dynabeads mRNA direct kit and an automated plate washer (Cytena C. Wash).

In vivo studies: In vivo studies were performed in the NSG-PiZ mouse model as described under Example 1. The different ASOs were used at 2.5 mg/kg, 5 mg/kg, and 10 mg/kg.

LC-MS levels: NSG-PiZ mouse serum samples were analysed by LC-MS/MS according to a protocol from Mayo Clinic with minor modifications (Chen et al., Clin. Chem., 2011; DOI: 10.1373/clinchem.2011.163006). Calibration standards and QC samples were prepared from alpha-1 antitrypsin (A1AT) isolated from human plasma (Athens Research & Technology, CAT #16160116091 MG) in surrogate matrix of 0.2% BSA in water. Calibration standard, QC, and serum sample dilutions (5 μL) were denatured with trifluoroethanol (TFE; 25 μL), diluted with 25 μL of 100 mM ammonium bicarbonate, and reduced with 10 μL of 50 mM dithiothreitol (DTT). After shaking at 55° C. for 30 minutes, the samples were treated with 10 μL of 150 mM iodoacetamide (IAA) and incubated for 30 min in the dark with shaking. The volumes were reduced to ˜25 μL by removing TFE under nitrogen for 10-15 min at 60° C. The samples were diluted with 45 μL of 100 mM ammonium bicarbonate and treated with 10 μL of 0.1 mg/mL trypsin at 37° C. overnight with shaking. The digestion was terminated by adding 50 μL of 2% formic acid solution, with simultaneous addition of isotopically labelled peptides: 10 nM of AVLTIDEK peptide (for wild-type A1AT quantification at the E342K, or ‘Z’, site) and 40 nM of prototypic SASLHLPK peptide (for total A1AT quantification). The samples were loaded on a Water Acquity Premier HSS T3 column (1.8 μm, 2.1×100 mm) and separated on a Water Acquity UPLC system with a gradient of 0.1% fluoric acid in water (mobile phase A) and acetonitrile (mobile phase B). Analytes were detected on a Sciex Triple Quad 5000 mass spectrometer, using positive ion electrospray with multiple reaction monitoring. Peaks corresponding to wild-type and total A1AT were identified by comparing the retention times to the respective labelled peptides. A calibration curve was constructed by calculating the area ratios for the unlabelled and labelled prototypic and wildtype Z peptide at each concentration of the A1AT standard. Wild-type and total A1AT levels in the experimental samples were determined by interpolation to the standard curve.

Statistical Analysis: One-way ANOVA relative to Day-7 vehicle with Bonferroni multiple-comparisons correction. *p<0.05; **0.001≤p<0.01; ***0.0001≤p<0.001; ****p<0.0001.

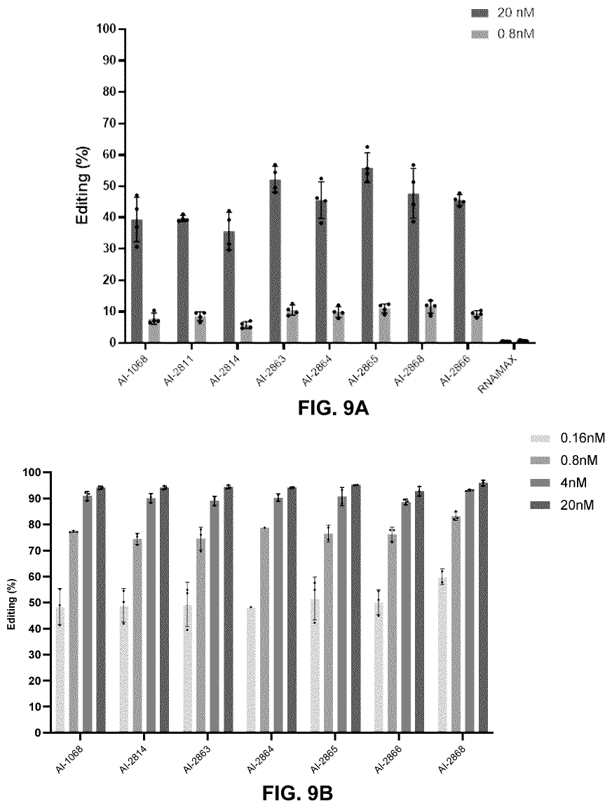

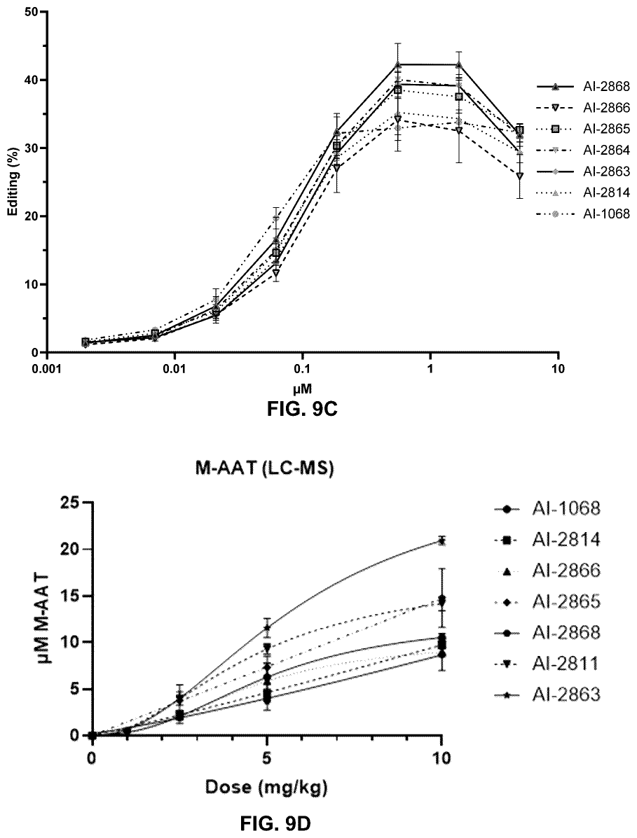

The different oligonucleotide constructs, their respective modification pattern and sequence are listed in Table 11. The results are shown in FIGS. 9A-9CD.

TABLE 11
Oligonucleotides.

| Construct | | Seq. | FIG. |
|---|---|---|---|
| (asymmetry) | Sequence (5′ to 3′ direction) | ID | No. |
| AI-1068 | mG&mC*mCfC&fC*mA*fG*mCmoeAfG*fC*moeT&fU*mCf | 4 | 9 |
| (25-1-8) | A*mG*fUmoe(MeC)*fC*moe(MeC)fU&mUmoeTfC*moeT* | | |
| | dC*dI&mUfC*moeG*mA*mU*mG&mG(Ser)(GalNac) | | |
| AI-2814 | mG&mCmoe(MeC)fC*fC*mA*fG*mCmoeAfG*fC*moeT&f | 6 | 9 |
| (25-1-8) | U*moe(MeC)fA*mG*mUmoe(MeC)fC*moe(MeC)fU*mUm | | |
| | oeTfC*moeT*dC*dI&mU*fC*moeGmA*mU*mG&mG(C7) | | |
| | (GalNac) | | |
| AI-2866 | mG&mCmC*fC*fC*mA*fG*mCmoeAfG*fC*moeT&mUmC*f | 10 | 9 |
| (25-1-8) | A*mG*mUmoe(MeC)*fC*moe(MeC)fU*mUmoeTfC*moeT* | | |
| | dC*dI&mU*fC*moeGfA*mU*mG&mG*(C7)(GalNac) | | |
| AI-2864 | mG&mCmC*fC*fC*mA*fG*mCmoeAfG*fC*moeT&mUmC*f | 8 | 9 |
| (25-1-8) | A*mG*mUmoe(MeC)*fC*moe(MeC)fU*mUmoeTfC*moeT* | | |
| | dC*dI&mUfC*moeGfA&mU*mG&mG*(C7)(GalNac) | | |
| AI-2865 | mG&mCmC*fC*fC*mA*fG*mCmoeAfG*fC*moeT&mUmC*f | 9 | 9 |
| (25-1-8) | A*mG*mUmoe(MeC)*fC*moe(MeC)fU*mUmoeTfC*moeT* | | |
| | dC*dI&mUfC*moeGfA*mU*mG&mG*(C7)(GalNac) | | |
| AI-2868 | mC&fC*fC*mA*fG*mCmoeAfG*fC*moeT&mUmC*fA*mG* | 14 | 9 |
| (23-1-8) | mUmoe(MeC)*fC*moe(MeC)fU*mUmoeTfC*moeT*dC*dI& | | |
| | mUfC*moeGmA*mU*mG&mG*(C7)(GalNac) | | |
| AI-2811 | mG&mCmC*fC*fC*mA*fG*mCmoeAfG*fC*moeT&fU*mC*f | 5 | 9 |
| (25-1-8) | A*mGmoeTmoe(MeC)fC*moe(MeC)fU*mUmoeTfC*moeT* | | |
| | dC*dI&mU*fC*moeGmAmU*mG&mG(C7)(GalNac) | | |
| AI-2863 | mG&mCmC*fC*fC*mA*fG*mCmoeAfG*fC*moeT&mUmC*f | 7 | 9 |
| (25-1-8) | A*mG*mUmoe(MeC)*fC*moe(MeC)fU*mUmoeTfC*moeT* | | |
| | dC*dI&mUfC*moeGmA*mU*mG&mG*(C7)(GalNac) | | |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine; GalNac = N-acetylgalactosamine; (C7) = C7 linker; (Ser) = serinol linker.

Transfection of Hela cells (FIG. 9A) demonstrated efficient delivery of all oligonucleotides. At a concentration of approximately 20 nM, all tested oligonucleotides exhibited editing efficacies of 40% or higher. Notably, transfection of primary PiZ-mouse hepatocytes further confirmed a dose-dependent increase in target editing, achieving editing levels of approximately 95% at 20 nM (FIG. 9B). Furthermore, it was demonstrated that each oligonucleotide was efficiently taken up by primary PiZ mouse hepatocytes to mediate editing (FIG. 9C). The specific atomic structures of the different oligonucleotides are summarised in FIG. 8 (AI-2863; compound (I)), and in FIG. 16 to FIG. 22 (compounds (II) to (VII), respectively).

Subsequently, the correlation between transfection efficacy and the improvement of M-AAT protein levels in vivo was evaluated. As shown in FIG. 9D, each oligonucleotide showed a general increase in target editing with rising oligonucleotide concentrations (2.5 mg/kg, 5 mg/kg, and 10 mg/kg). By day 21, a dose-dependent increase in target editing and M-AAT serum levels was observed, with higher concentrations leading to a general rise in M-AAT level (FIG. 9D). Specifically, AI-2811, AI-2865, and AI-2863 all led to significantly higher M-AAT levels. The statistical data are summarised in Table 12.

TABLE 12
Significance of change in M-AAT serum level
for each oligonucleotide relative to AI-1068.

| Construct | 2.5 mg/kg | 5 mg/kg | 10 mg/kg |
|---|---|---|---|
| AI-2814 | ns | ns | ns |
| AI-2866 | ns | ns | ns |
| AI-2865 | * | ns | * |
| | (higher) | | (higher) |
| AI-2868 | ns | * | ns |
| | | (lower) | |
| AI-2811 | * | * | * |
| | (higher) | (higher) | (higher) |
| AI-2863 | * | **** | **** |
| | (higher) | (higher) | (higher) |

Overall, these data confirm that AI-2863 consistently achieves high target editing efficiency and elevated M-AAT levels.

### Example 8. Oligonucleotide AI-2863 and Other Oligonucleotides Show a Significant and Superior Increase in Target Tissue Exposure Compared to Control Sequence

The pharmacokinetic properties of oligonucleotides are largely influenced by the backbone chemistry and characterized by distribution from plasma to tissue and subsequent redistribution from tissue. Upon administration, oligonucleotides are systemically distributed across various tissues, including the liver, kidneys, and bone marrow. To evaluate whether different modification patterns affected each oligonucleotide's availability in the relevant target tissue, exposure levels of each oligonucleotide were assessed in vivo.

Tissue exposure: hELISA (hybridization enzyme-linked immunosorbent assay) was used to measure the oligonucleotide tissue exposure. Flash frozen mouse liver tissue was lysed in lysis buffer supplemented with Proteinase K (Elabscience, E-IR-R109U) using a bead homogenizer (Bead Mill Max, VWR) with 1.4 mm ceramic beads. The lysates were incubated at 55° C. for 1 h with gently shaking and afterwards used in the assay. The capture and detection probes were custom synthesized as a 12 mer or 15 mer DNA oligonucleotide complementary to the analyte (Integrated DNA Technologies). The capture probe was conjugated to biotin at the 3′ terminus, while the detection probe has digoxigenin conjugation at the 5′ terminus. The capture probe was attached to a NeutrAvidin-coated 96-well plate (Pierce, 15510) via biotin. A 2-fold calibration curve was setup from 100 ng/ml by diluting the oligonucleotide into matrix. Diluted samples and standards were mixed with the detection probe for hybridization. Samples were transferred into blocked ELISA plates and incubated to allow the binding of the analyte to the capture probe. After washing, an anti-digoxigenin-AP (Sigma-Aldrich, 11093274910) was added to the samples. After washing to remove unligated antibody, an alkaline phosphatase substrate (AttoPhos-Promega, S1000) was added and fluorescence intensity was measured at excitation of 435 nm and emission of 555 nm using a plate reader (SpectraMax i3x, Molecular Device). The oligonucleotide content in the samples was calculated according to standard curve by four-parameter regression and expressed as mg per gram of tissue.

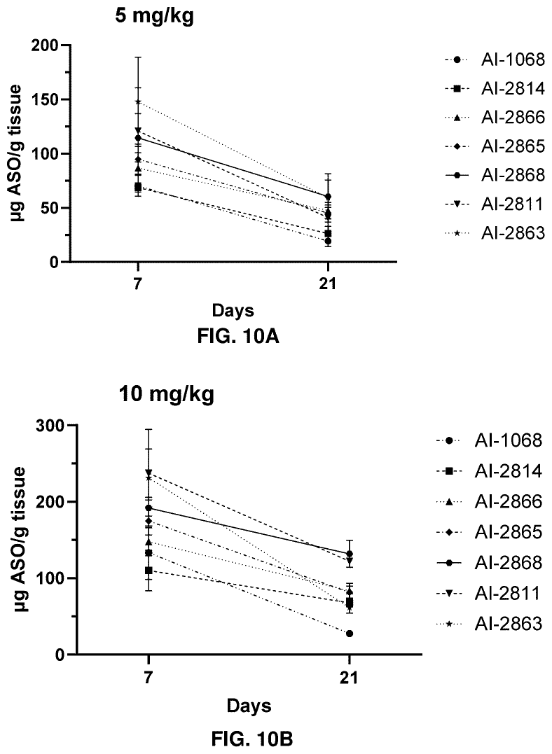

Most oligonucleotide constructs as listed in Table 11 of Example 7 were used. Oligonucleotides were administered at a dose of 5 mg/kg or 10 mg/kg. The results are shown in FIGS. 10A-10B. The statistical data are expressed relative to AI-1068 and summarised in Table 13.

TABLE 13
Significance of ASO exposure in target relative to AI-1068.

| | |
|---|---|
| Day 7 | Day 21 |

| Construct | 5 mg/kg | 10 mg/kg | 5 mg/kg | 10 mg/kg |
|---|---|---|---|---|
| AI-2814 | ns | Ns | ns | *** |
| | | | | (higher) |
| AI-2866 | ns | Ns | ** | **** |
| | | | (higher) | (higher) |
| AI-2865 | ns | Ns | ** | **** |
| | | | (higher) | (higher) |
| AI-2868 | ns | Ns | **** | **** |
| | | | (higher) | (higher) |
| AI-2811 | * | ** | * | **** |
| | (higher) | (higher) | (higher) | (higher) |
| AI-2863 | ** | * | **** | ** |
| | (higher) | (higher) | (higher) | (higher) |

As illustrated in FIG. 10A, oligonucleotide tissue exposure generally decreased at a dose of 5 mg/kg between day 7 and day 21. A comparable trend was observed at a dose of 10 mg/kg, as shown in FIG. 10B. Nevertheless, the amount of ASO per tissue recovered was about 2 to 3-fold higher in the 10 mg/kg group than in the 5 mg/kg group. While all oligonucleotides showed higher tissue exposure than AI-1068, only AI-2811 and AI-2863 showed significantly higher exposure by day 7 for both concentrations (Table 13). On the other hand, by day 21, almost all oligonucleotides showed a significant increase when compared to AI-1068 (Table 13).

The results demonstrate that all oligonucleotides enhanced tissue exposure, with AI-2863 showing superior target tissue exposure compared to other oligonucleotides tested. These data further confirm that incorporating specific chemical modifications tailored to each oligonucleotide enhances target editing and M-AAT levels while also improving tissue exposure.

### Example 9. New Oligonucleotides Show Better Potency and Durability than AI-1068 in B6 AATD Mouse Model

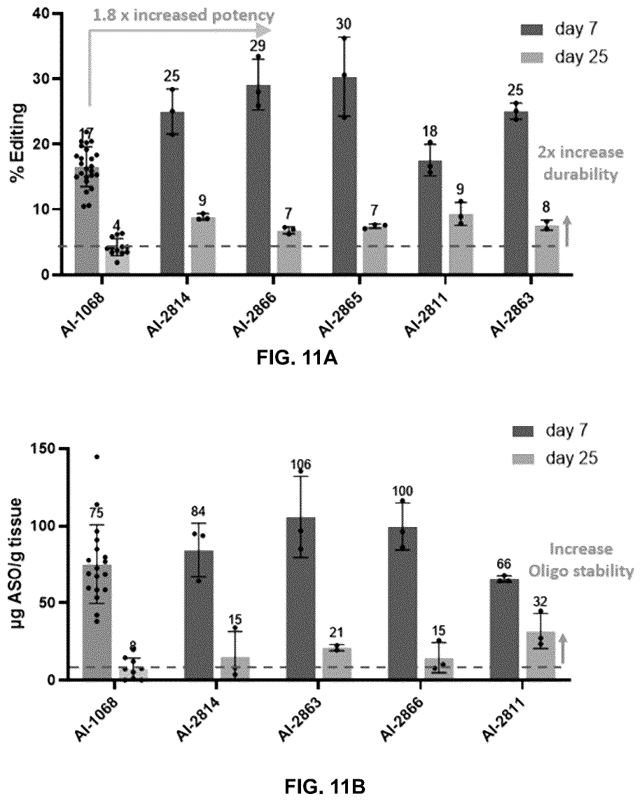

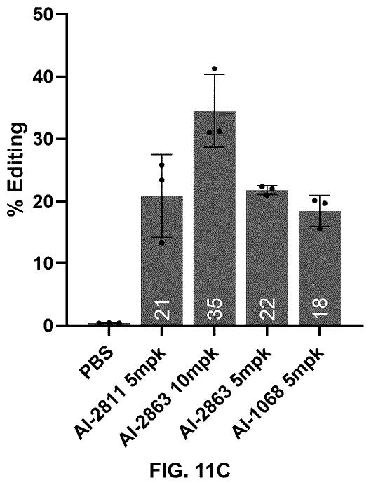

Given the diverse chemical modification patterns of each oligonucleotide, in vivo potency and durability were subsequently assessed for each. Target editing and exposure levels were evaluated in the liver for each oligonucleotide. Most oligonucleotide constructs as listed in Table 11 were used. The results are shown in FIGS. 11A-11C.

hELISA: hELISA (hybridization enzyme-linked immunosorbent assay) was used to measure the oligonucleotide content in the liver. Flash frozen mouse liver tissue was lysed in lysis buffer supplemented with Proteinase K (Elabscience, E-IR-R109U) using a bead homogenizer (Bead Mill Max, VWR) with 1.4 mm ceramic beads. The lysates were incubated at 55° C. for 1 h with gently shaking and afterwards used in the assay. The capture and detection probes were custom synthesized as a 12 mer or 15 mer DNA oligonucleotide complementary to the analyte (Integrated DNA Technologies). The capture probe was conjugated to biotin at the 3′ terminus, while the detection probe has digoxigenin conjugation at the 5′ terminus. The capture probe was attached to a NeutrAvidin-coated 96-well plate (Pierce, 15510) via biotin. A 2-fold calibration curve was setup from 100 ng/ml by diluting the oligonucleotide into matrix. Diluted samples and standards were mixed with the detection probe for hybridization. Samples were transferred into blocked ELISA plates and incubated to allow the binding of the analyte to the capture probe. After washing, an anti-digoxigenin-AP (Sigma-Aldrich, 11093274910) was added to the samples. After washing to remove unligated antibody, an alkaline phosphatase substrate (AttoPhos-Promega, S1000) was added and fluorescence intensity was measured at excitation of 435 nm and emission of 555 nm using a plate reader (SpectraMax i3x, Molecular Device). The oligonucleotide content in the samples was calculated according to standard curve by four-parameter regression and expressed as mg per gram of tissue.

As shown in FIG. 11A, there was an increase in target editing (%) by day 7 for all the oligonucleotides tested. This increase was particularly prominent for AI-2814, AI-2866, AI-2865, and AI-2863. For AI-2866 and AI-2865, potency increased by approximately 1.8-fold relative to AI-1068 by day 7, while AI-2814 and AI-2863 showed an approximate 1.5-fold increase. A similar, though lower, trend was observed by day 25. All oligonucleotides showed higher editing (%) than AI-1068. Notably, AI-2814, AI-2811 and AI-2863 demonstrated an at least 2-fold increase in target editing compared to AI-1068 (FIG. 11A).

As shown in FIG. 11B, oligonucleotide content in the liver was highest for AI-2863 on day 7 (about 1.4-fold higher than AI-1068). This was followed by AI-2866, AI-2814, and AI-2811 on day 7. However, by day 25, AI-2811 showed the highest level of ASO per tissue sample, confirming increased oligonucleotide stability. The target editing results were subsequently confirmed in a repeat study. As shown in FIG. 11C, AI-2863 demonstrated approximately 35% editing efficacy, confirming it was still more effective than AI-1068 (see, Example 2, FIG. 2B).

Again, the results show that systematic stabilization of distinct positions within the oligonucleotide with specific linkage modifications (e.g., PS linkages), 2′MOE and 2′F/2′OMe modifications improve oligonucleotide potency and durability.

### Example 10. New Oligonucleotides Show No Detectable Organ Toxicity and Remain Safe at High Doses In Vivo

Analogous to Example 3, the toxicity profile of the different, newly generated oligonucleotides was determined in vivo. The experimental set up was identical to that used in Example 3. Briefly, in vivo toxicity parameters were measured both pre-administration and after a 100 mg/kg dose was given to B6-PiZ mice. Samples were collected pre-dose, 4 h post-dosing, on day 1 and day 7 post administration, and subsequently assessed for liver (ALT and AST) and kidney function (creatinine and BUN) parameters as well as for immunogenetic responses.

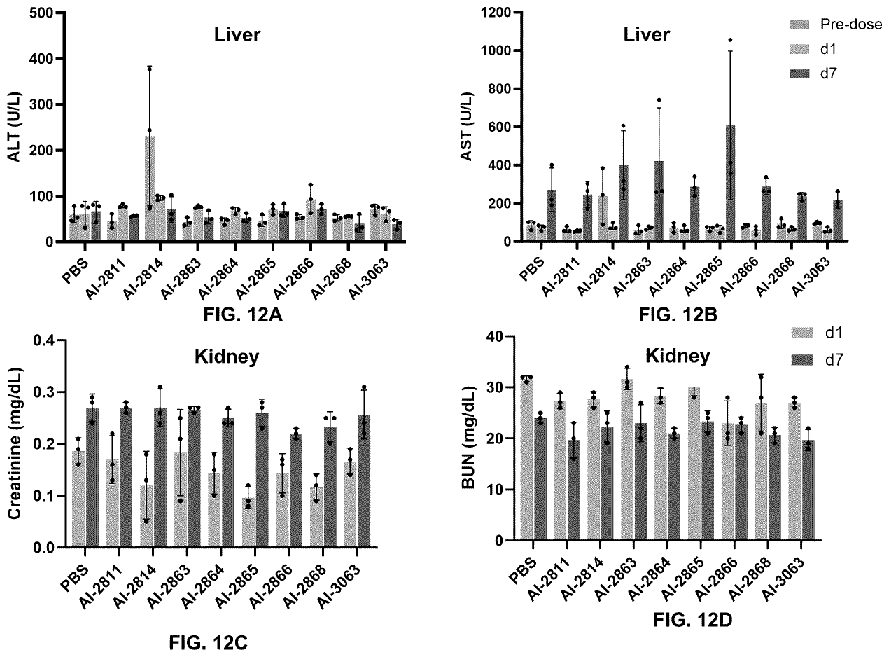

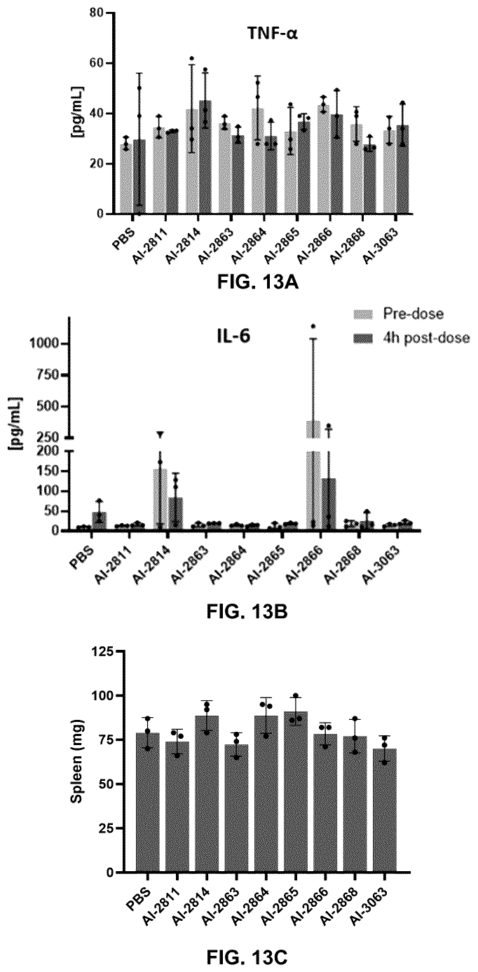

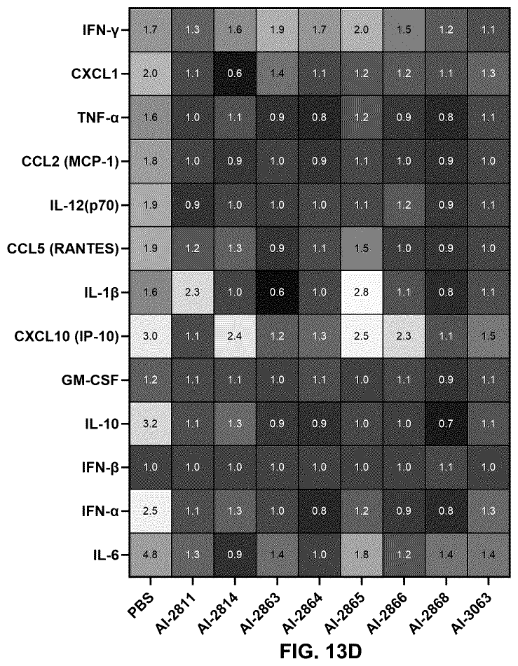

The different oligonucleotides, their respective modification pattern and sequence are listed in Table 14. The results are shown in FIGS. 12A-12D (organ toxicity) and FIGS. 13A-13D (immunogenicity).

TABLE 14
Oligonucleotides.

| Construct | | Seq. | FIG. |
|---|---|---|---|
| (asymmetry) | Sequence (5′ to 3′ direction) | ID | No. |
| Al-2811 | mG&mCmC*fC*fC*mA*fG*mCmoeAfG*fC*moeT&fU*mC* | 5 | 12, 13 |
| (25-1-8) | fA*mGmoeTmoe(MeC)fC*moe(MeC)fU*mUmoeTfC*mo | | |
| | eT*dC*dI&mU*fC*moeGmAmU*mG&mG(C7)(GalNac) | | |
| AI-2814 | mG&mCmoe(MeC)fC*fC*mA*fG*mCmoeAfG*fC*moeT& | 6 | 12, 13 |
| (25-1-8) | fU*moe(MeC)fA*mG*mUmoe(MeC)fC*moe(MeC)fU*mU | | |
| | moeTfC*moeT*dC*dI&mU*fC*moeGmA*mU*mG&mG(C7) | | |
| | (GalNac) | | |
| AI-2863 | mG&mCmC*fC*fC*mA*fG*mCmoeAfG*fC*moeT&mUm | 7 | 12, 13 |
| (25-1-8) | C*fA*mG*mUmoe(MeC)*fC*moe(MeC)fU*mUmoeTfC*m | | |
| | oeT*dC*dI&mUfC*moeGmA*mU*mG&mG*(C7)(GalNac) | | |
| AI-2864 | mG&mCmC*fC*fC*mA*fG*mCmoeAfG*fC*moeT&mUm | 8 | 12, 13 |
| (25-1-8) | C*fA*mG*mUmoe(MeC)*fC*moe(MeC)fU*mUmoeTfC*m | | |
| | oeT*dC*dI&mUfC*moeGfA&mU*mG&mG*(C7)(GalNac) | | |
| AI-2865 | mG&mCmC*fC*fC*mA*fG*mCmoeAfG*fC*moeT&mUm | 9 | 12, 13 |
| (25-1-8) | C*fA*mG*mUmoe(MeC)*fC*moe(MeC)fU*mUmoeTfC*m | | |
| | oeT*dC*dI&mUfC*moeGfA*mU*mG&mG*(C7)(GalNac) | | |
| AI-2866 | mG&mCmC*fC*fC*mA*fG*mCmoeAfG*fC*moeT&mUm | 10 | 12, 13 |
| (25-1-8) | C*fA*mG*mUmoe(MeC)*fC*moe(MeC)fU*mUmoeTfC*m | | |
| | oeT*dC*dI&mU*fC*moeGfA*mU*mG&mG*(C7)(GalNac) | | |
| AI-2868 | mC&fC*fC*mA*fG*mCmoeAfG*fC*moeT&mUmC*fA*mG* | 14 | 12, 13 |
| (23-1-8) | mUmoe(MeC)*fC*moe(MeC)fU*mUmoeTfC*moeT*dC* | | |
| | dI&mUfC*moeGmA*mU*mG&mG*(C7)(GalNac) | | |
| AI-3063 | mG&mCmoe(MeC) *fC*fC*mA*fG*mCmoeAfG*fC*moeT | 11 | 12, 13 |
| (25-1-8) | &mUmoe(MeC)*fA*mG*mUmoe(MeC)*fC*moe(MeC)fU* | | |
| | mUmoeTfC*moeT*dC*dI&mU*fC*moeGfA*mU*mG&mo | | |
| | eG*(C7)(GalNac) | | |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine; GalNac = N-acetylgalactosamine; (C7) = C7 linker.

As show in FIGS. 12A-12D, all oligonucleotides showed similar levels in ALT expression 1 day and 7 days post-treatment. These levels were similar to the PBS only control (FIG. 12A). Likewise, there was no significant change in AST expression 1 day post treatment. However, by day 7, there was a general increase in AST levels for some of the oligonucleotides (FIG. 12B).

As for kidney function, no significant change in creatinine or BUN expression levels were detected 1 day or 7 days post treatment when compared to control (FIGS. 12C-12D).

Overall, no significant elevation in liver and/or kidney toxicity biomarkers was detected 1 day or 7 days post administration of a very high ASO dose (100 mg/kg).

Subsequently, as with Example 3, the immunogenicity of each oligonucleotide was determined by measuring the level of pro-inflammatory cytokines TNF-α and IL-6. Additionally, as infections, liver disease and injuries can cause the spleen to swell and become larger, changes in the weight of the spleen were also determined. The results are shown in FIGS. 13A-13D.

As shown in FIG. 13A, all oligonucleotides showed TNF-α expression levels that were similar to control. There was no significant change in TNF-α expression for any of the oligonucleotides tested. For IL-6, an overall increase could be observed for the AI-2814 and AI-2866 candidates, both prior to administration and 4 hours post-administration (FIG. 13B). In general, no acute immune reaction could be observed in mice and all cytokines showed less than 3-fold increase 4 hours after treatment (the results are normalized to baseline levels) (FIG. 13D). Also, there was no significant change in spleen weight (FIG. 13C), suggesting the absence of any underlying disorder or inflammatory response. The results were subsequently confirmed for the oligonucleotides in a repeat study showing no significant change in cytokine expression levels.

These results not only confirm the data of Example 3, but further demonstrate that also the new and further oligonucleotides show no toxicity and remain safe at high at high concentrations. These data again confirm the safety profile of the different oligonucleotides.

### Example 11. New Oligonucleotides Show No Detectable Signs of Cytotoxicity and No Immune Activation in Human PBMCs

Cytotoxicity of the new oligonucleotides was assessed as described in Example 4. The protocols are listed in Example 4. Transfection agent without ASO (UTC) and AI-0137-003 (Inclisiran Fluorochem) was used as negative control. AI-0139-001 was used as positive control (see, Table 7 for sequence). Cellular toxicity was assessed in vitro for the different oligonucleotides by determining the extracellular ATP release content, LDH release and caspase activation pathway.

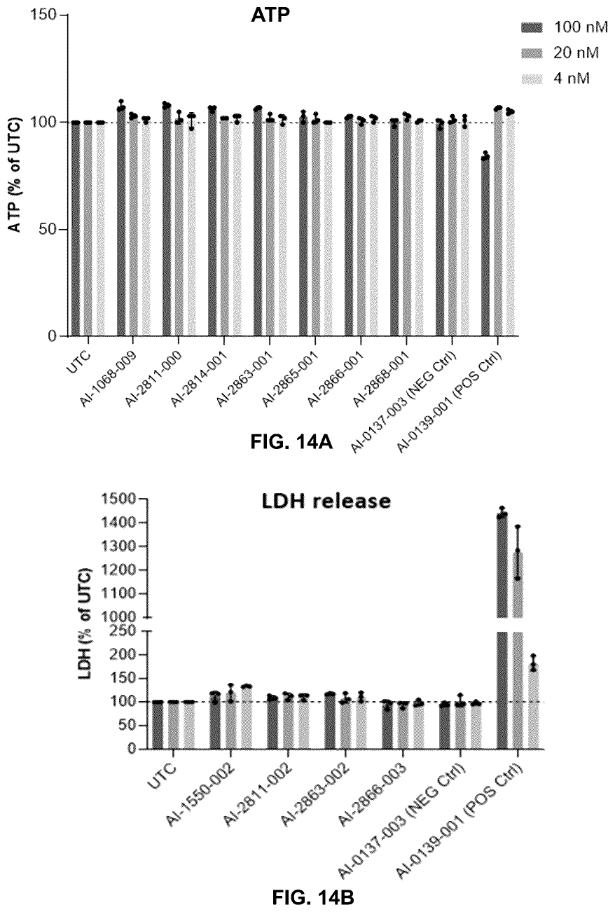

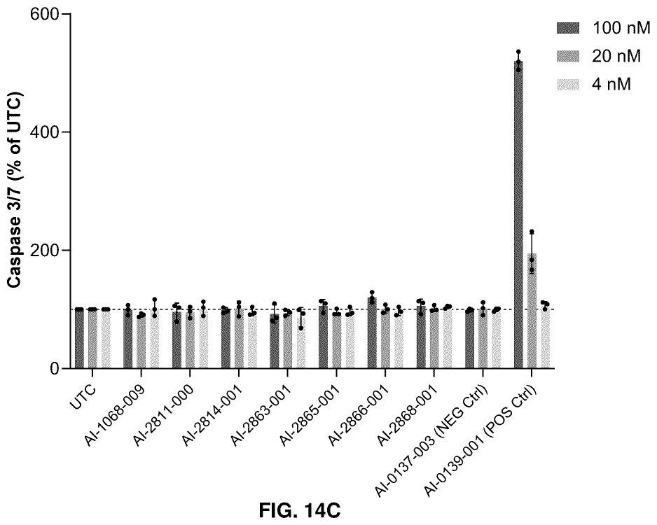

As shown in FIGS. 14A-14C, there was no signification change in ATP content, LDH release and/or caspase 3/7 activation at the different concentrations tested (4 nM, 20 nM, and 100 nM). These data demonstrate that the newly generated and identified lead oligonucleotides show no detectable signs of cytotoxicity.

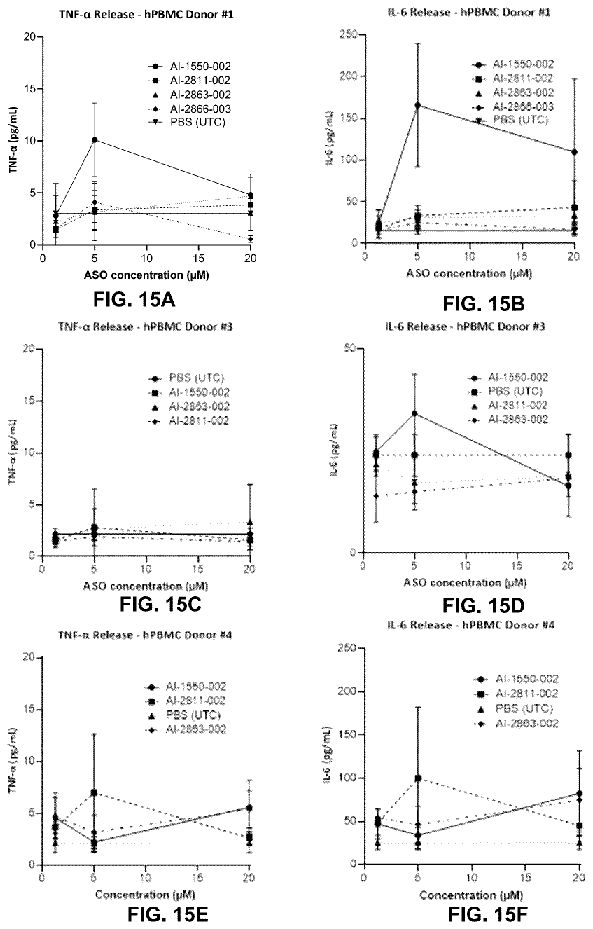

Further, the immunotoxicity profile of the different oligonucleotides was assessed as described in Example 5. The protocol was as described for Example 5. PBS was used as control. The immunotoxic potential of the oligonucleotides was evaluated by assessing the changes in pro-inflammatory cytokine serum levels (TNFα, and IL-6) in hPBMCs. As shown in FIGS. 15A-15F, no immune activation was detected for any of the oligonucleotides. That is, there was no significant change in TNF-α and/or IL-6 expression in any of the three donors tested.

### Example 12. Free Uptake of C₂-C₆ Sulfonylphosphoramidate Internucleoside Linkage Modified ASOs in PiZ Primary Mouse Hepatocytes

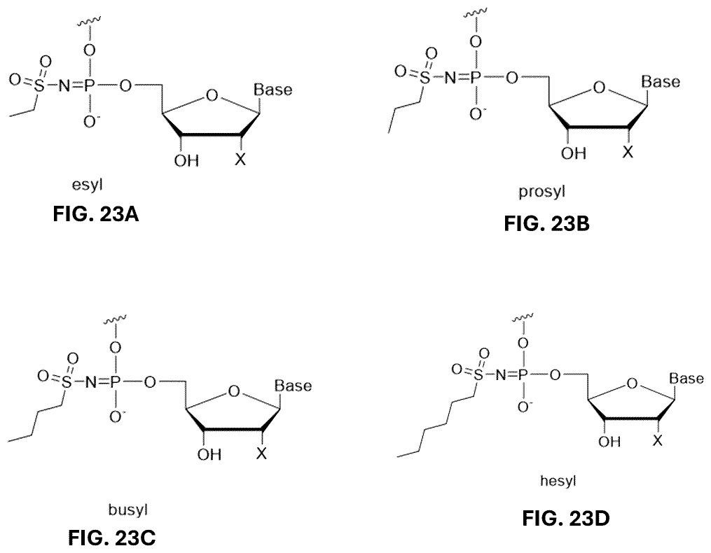

Single-stranded oligonucleotides can efficiently be taken up by living cells without the use of transfection reagents. This phenomenon called gymnosis enables the sequence-specific silencing of target genes in various types of cells. To determine the in vitro uptake efficacy of the different oligonucleotides that carry distinct internucleoside linkage modifications at identical positions, free ASO uptake was assessed in PiZ primary mouse hepatocytes. Specifically, the ASO constructs AI4486, AI-4487, AI-4488 and AI-4543 were generated, carrying either ethanesulfonyl (esyl), 1-propanesulfonyl (propyl), 1-butanesulfonyl (busyl) or 1-hexanesulfonyl (hesyl or hexyl) internucleoside linkages respectively (see, FIGS. 23A-23D for linkages). Each ASO contains the respective sulfonylphosphoramidate internucleoside linkage at the same positions as the mesylated AI-2863 ASO, namely at positions +24, +13, −2, and −8.

Oligonucleotide synthesis: Oligonucleotides were synthesised as generally described under general protocols above. Nucleoside phosphoramidates (e.g., mesyl, esyl, prosyl, busyl or hesyl linkages) were incorporated using standard solid-phase oligonucleotide synthesis.

Cell culture, free uptake and mRNA isolation in primary mouse hepatocytes: Freshly isolated primary mouse hepatocytes from transgenic hSERPINA1 E342K (PiZ) mice, were plated in 96-well collagen-coated plates (Greiner) at a density of 2.5×10⁴ cells per well (100 μL per well) in DMEM low glucose (Gibco) supplemented with 10% FBS (Gibco) and 1% penicillin/streptomycin (Gibco) for 4 hours, then they were cultured in William's E Medium (Gibco) supplemented with 1% GlutaMAX™ (Gibco) and 1% penicillin/streptomycin for 24 hours. The hepatocytes were cultured under standard culture conditions at 37° C., 5% CO₂ and in a humidified atmosphere. With the media change, cells were treated with different concentrations of ASOs in 1×PBS (Gibco) for the free uptake. Cells were washed with PBS and harvested 24 hours after ASO treatment in 125 μl/well lysis buffer (Dynabeads mRNA direct kit, Invitrogen). Lysates of 96-well plates were transferred to a 384-plate and mRNA was isolated using the Dynabeads mRNA direct kit and an automated plate washer (Cytena C. Wash).

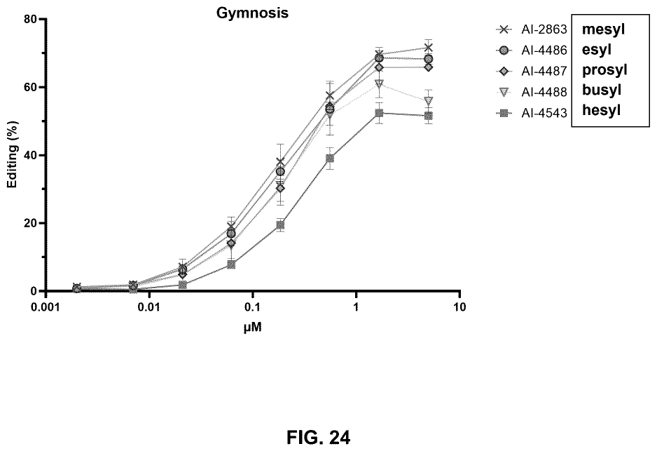

The different oligonucleotide constructs, their respective modification pattern and sequence are listed in Table 15. The results of Example 12 are shown in FIG. 24. Oligonucleotide AI-2863, having mesyl linkages at positions +24, +13, −2, −8, served as control.

TABLE 15
Oligonucleotide constructs and modifications.

| | | Seq. | FIG. |
|---|---|---|---|
| Construct | Sequence (5′ to 3′ direction) | ID | No. |
| AI-2863 | MesyI: “+24, +13, −2, -8”; PO 23, 17, 16, 12, | 7 | 24 |
| (25-1-8) | 8, 5, 3, 2, −3, −5 | | |
| | mG&mCmC*fC*fC*mA*fG*mCmoeAfG*fC*moeT&mUmC* | | |
| | fA*mG*mUmoe(MeC) *fC*moe(MeC)fU*mUmoeTfC*moeT* | | |
| | dC*dI&mUfC*moeGmA*mU*mG&mG*(C7)(GalNac) | | |
| AI-4486 | EsyI: “+24, +13, −2, −8” | 21 | 24 |
| (25-1-8) | mG[>e]mCmC*fC*fC*mA*fG*mCmoeAfG*fC*moeT[>e]mU | | |
| | mC*fA*mG*mUmoe(MeC)*fC*moe(MeC)fU*mUmoeTfC*m | | |
| | oeT*dC*dI[>e]mUfC*moeGmA*mU*mG[>e]mG*(C7)(GalN | | |
| | ac) | | |
| AI-4487 | Prosyl: “+24, +13, −2, −8” | 22 | 24 |
| (25-1-8) | mG[>p]mCmC*fC*fC*mA*fG*mCmoeAfG*fC*moeT[>p]mU | | |
| | mC*fA*mG*mUmoe(MeC)*fC*moe(MeC)fU*mUmoeTfC*m | | |
| | oeT*dC*dI[>p]mUfC*moeGmA*mU*mG[>p]mG*(C7) | | |
| | (GalNac) | | |
| AI-4488 | Busyl: “+24, +13, −2, −8” | 23 | 24 |
| (25-1-8) | mG[>b]mCmC*fC*fC*mA*fG*mCmoeAfG*fC*moeT[>b]mU | | |
| | mC*fA*mG*mUmoe(MeC) *fC*moe(MeC)fU*mUmoeTfC*mo | | |
| | eT*dC*dI[>b]mUfC*moeGmA*mUmG[>b]mG*(C7)(GalNac) | | |
| AI-4543 | Hesyl: “+24, +13, −2, −8” | 24 | 24 |
| (25-1-8) | mG[>h]mCmC*fC*fC*mA*fG*mCmoeAfG*fC*moeT[>h]mU | | |
| | mC*fA*mG*mUmoe(MeC)*fC*moe(MeC)fU*mUmoeTfC*m | | |
| | oeT*dC*dI[>h]mUfC*moeGmA*mU*mG[>h]mG*(C7)(Gal | | |
| | Nac) | | |

d = 2′-H (deoxyribose; DNA); * = phosphorothioate (PS); & = (mesyl) methanesulfonyl; [>e] = esyl linkage; [>p] = prosyl linkage; [>b] = busyl linkage; m = 2′-OMe; f = 2′-fluoro; moe = 2′-O-methoxyethyl (2′-MOE); I = inosine; MeC = 5′-methylcytidine; GalNac = N-acetylgalactosamine; [>e] = esyl linkage; [>p] = prosyl linkage; [>b] = busyl linkage; [>h] = hesyl linkage.

As shown in FIG. 24, an increase in target editing (%) was observed with rising oligonucleotide concentrations. Maximum target editing was achieved at concentrations ranging from 1 to 10 UM for each of the ASOs tested.

Overall, these data demonstrate that substituting mesyl linkages with esyl, prosyl, busyl, or hesyl linkages at the same positions also facilitates efficient uptake and SERPINA1 target editing.

### Example 13. C2-C6 Sulfonylphosphoramidate Internucleoside Linkages Lead to Efficient SERPINA Target Editing In Vivo

To further determine the effect of the different types of alkyl internucleoside linkage modifications, the different oligonucleotide constructs as used in Example 17 were tested for their in vivo editing efficacy.

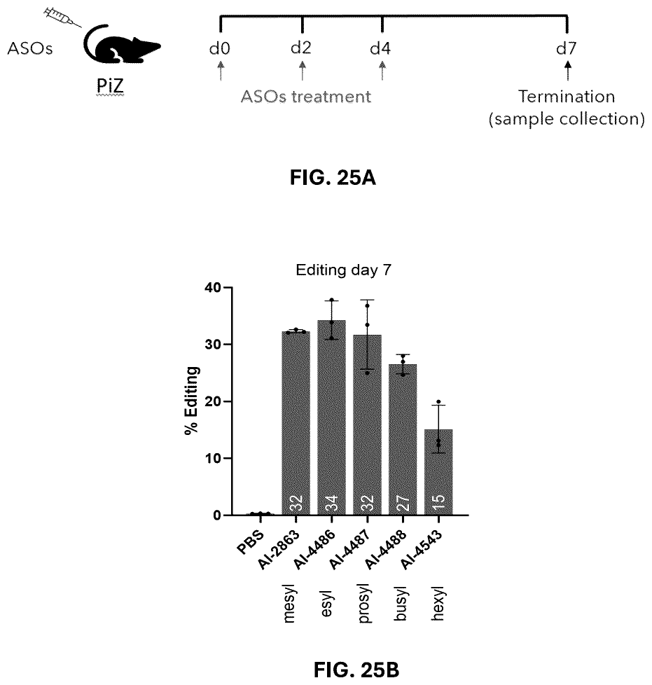

In vivo studies: PiZ mice were administered a dose of 10 μm/kg on day 0, day 2, and day 4 (a total dose of 3×10 mg/kg) of either AI-2863, AI4486, AI-4487, AI-4488 or AI-4543 as outlined in FIG. 25A and summarised in Table 16. Organs and serum were harvest on day 7 and analysed for target editing. Number of animals per treatment group n=3. PBS served as negative control. The different oligonucleotides used in Example 12 and their respective sequences and chemical modifications are listed in Table 17. The results are presented in FIG. 25B.

TABLE 16
Summary of group cohorts receiving the different test constructs.

| Group no. | 1 | 2 | 3 | 4 | 5 | 6 |
|---|---|---|---|---|---|---|
| Group | PBS | AI-2863 | AI-4486 | AI-4487 | AI-4488 | AI-4543 |
| name | | | | | | |
| size [nt] | — | 34 nt | 34 nt | 34 nt | 34 nt | 34 nt |
| Dose | — | 3 × 10 | 3 × 10 | 3 × 10 | 3 × 10 | 3 × 10 |
| (mg/kg) | | | | | | |
| Termination | d 7 | d 7 | d 7 | d 7 | d 7 | d 7 |

TABLE 17
Oligonucleotide constructs and modifications.

| | | Seq. | FIG. |
|---|---|---|---|
| Construct | Sequence (5′ to 3′ direction) | ID | No. |
| AI-2863 | Mesyl: “+24, +13, −2, −8”; PO 23, 17, 16, | 7 | 25 |
| (25-1-8) | 12, 8, 5, 3,2, −3, −5 | | |
| | mG&mCmC*fC*fC*mA*fG*mCmoeAfG*fC*moeT&mUmC* | | |
| | fA*mG*mUmoe(MeC) *fC*moe(MeC)fU*mUmoeTfC*mo | | |
| | eT*dC*dI&mUfC*moeGmA*mU*mG&mG*(C7)(GalNac) | | |
| AI-4486 | Esyl: “+24, +13, −2, −8” | 21 | 25 |
| (25-1-8) | mG[>e]mCmC*fC*fC*mA*fG*mCmoeAfG*fC*moeT[>e]mU | | |
| | mC*fA*mG*mUmoe(MeC)*fC*moe(MeC)fU*mUmoeTfC*m | | |
| | oeT*dC*dI[>e]mUfC*moeGmA*mU*mG[>e]mG*(C7)(Gal | | |
| | Nac) | | |
| AI-4487 | Prosyl: “+24, +13, −2, −8” | 22 | 25 |
| (25-1-8) | mG[>p]mCmC*fC*fC*mA*fG*mCmoeAfG*fC*moeT[>p]mU | | |
| | mC*fA*mG*mUmoe(MeC)*fC*moe(MeC)fU*mUmoeTfC*m | | |
| | oeT*dC*dI[>p]mUfC*moeGmA*mU*mG[>p]mG*(C7)(Gal | | |
| | Nac) | | |
| AI-4488 | Busyl: “+24, +13, −2, −8” | 23 | 25 |
| (25-1-8) | mG[>b]mCmC*fC*fC*mA*fG*mCmoeAfG*fC*moeT[>b]mU | | |
| | mC*fA*mG*mUmoe(MeC)*fC*moe(MeC)fU*mUmoeTfC*m | | |
| | oeT*dC*dI[>b]mUfC*moeGmA*mU*mG[>b]mG*(C7)(Gal | | |
| | Nac) | | |
| AI-4543 | Hesyl: “+24, +13, −2, −8” | 24 | 25 |
| (25-1-8) | mG[>h]mCmC*fC*fC*mA*fG*mCmoeAfG*fC*moeT[>h]mU | | |
| | mC*fA*mG*mUmoe(MeC)*fC*moe(MeC)fU*mUmoeTfC*m | | |
| | oeT*dC*dI[>h]mUfC*moeGmA*mU*mG[>h]mG*(C7)(Gal | | |
| | Nac) | | |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine; GalNac = N-acetylgalactosamine; [>e] = esyl linkage; [>p] = prosyl linkage; [>b] = busyl linkage; [>h] = hesyl linkage.

As shown in FIG. 25B, there was good in vivo target editing in the liver by day 7 in animals that had received oligonucleotide AI-2863 (mesyl linkages), AI-4486 (esyl linkages), or AI-4487 (prosyl linkages). Each of these test oligonucleotides achieved a target editing efficacy of approximately 30%. In animals treated with ASO AI-4488 or AI-4543, there was a gradual decrease in target editing efficacy (%).

Overall, these results serve as a proof of concept to show that ASOs carrying esyl and prosyl linkages can be designed to mediate efficient A-to-I editing of a target RNA.

Overall, the consolidated results show that AI-2863 had significantly better efficacy (target editing and M-AAT levels) than AI-1550 (corresponding to AI-1068 but containing a C7 linker) in vitro and in vivo across both B6 and NSG mice across all doses at early timepoint. Specifically, AI-2863 had improved durability than AI-1550 which resulted in higher target editing and M-AAT levels in NSG and BL6 mice at day 21 and day 25 respectively. AI-2863 was well tolerated in two independent high dose 100 mg/kg B6 mouse studies with no elevation in liver, kidney or inflammation biomarkers. N₀ in vitro toxicity was detected. Further, it was shown that, with the exception of a slight increase in cytokines for AI-1550 (donor #1), no cytokine elevation was detected for any of the oligonucleotides tested across any of the three hPBMC donors. Overall, these data show that the A-to-I editing efficacy of the SERPINA1-specific oligonucleotides and be modulated or improved depending on the combination of features including asymmetry, internucleoside linkage modifications and 2′ sugar modifications. The specific atomic structures of the different oligonucleotides are shown in FIG. 8 (AI-2863; compound (I)), and FIG. 16 to FIG. 22 (compounds (II) to (VIII), respectively). Also, the inventors were able to show the oligonucleotides disclosed herein are not limited to specific types of sulfonylphosphoramidate internucleoside linkages. That is, introducing mesyl, esyl and/or prosyl linkages led to efficient editing of the SERPINA1 target RNA in vivo (FIGS. 25A-25B).

### Example 14. Interferon Treatment Only Provides a Small Increase in Oligonucleotide Editing in Hepatocytes

iPSC-derived human hepatocytes carrying the mutation A1ATD E342K (DefiniGEN, UK) were plated into collagen-coated 96-well plates at a density of 66,000 cells per well. Plating media and culturing media were provided by DefiniGEN. Cells were maintained under hypoxic conditions for 9 days. Medium was changed on Days 2, 4, 7, and 9. After culturing the cells for 9 days, cells were transfected with indicated concentrations of AI-0991 (1 nM-100 nM) using Lipofectamine RNAiMAX (Invitrogen, Carlsbad, CA) according to the manufacturer's instructions. Cells were treated with 1U/μL Interferon-a as indicated. Forty-eight hours after treatment, cells were lysed and the RNA was isolated with the Dynabeads mRNA purification kit (Invitrogen, Carlsbad, CA) according to manufacturer's instruction for subsequent reverse transcription, PCR and NGS amplicon sequencing.

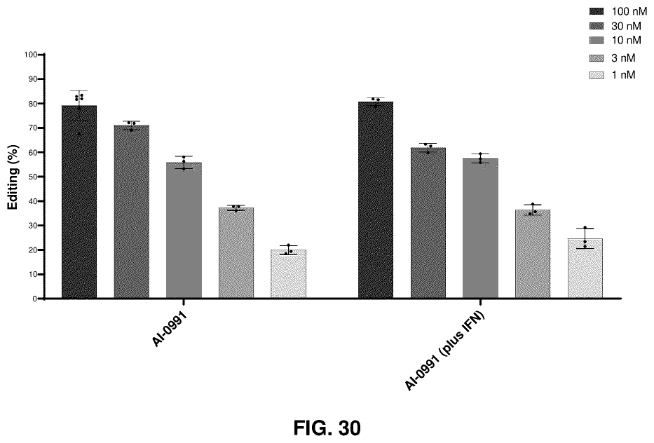

As seen in FIG. 30, treatment with AI-0991 produced nearly identical levels of editing compared to treatment with AI-0991 combined with IFN-α at every dose. This indicates that IFN is not necessary for editing.

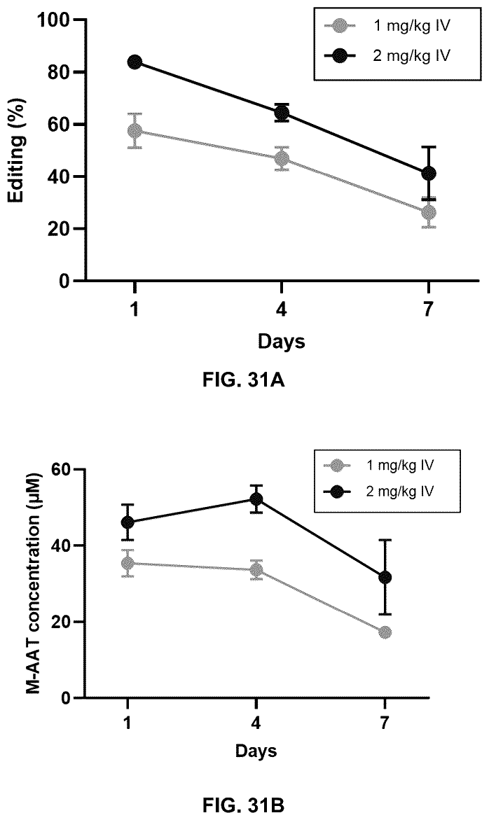

TABLE 18
Oligonucleotide constructs and modifications.

| | | Seq. | Figure |
|---|---|---|---|
| Construct | Sequence (5′ to 3′ direction) | ID | No. |
| AI-0991 | mG&mC*mCfC&fC*mA*fG*mCmoeAfG*fC*moeT&fU*mCf | 25 | FIG. 30, |
| (25-1-8) | A*mG*fUmoe(MeC)*fC*moe(MeC)fU&mUmoeTfC*moeT* | | FIGs. 31A |
| | dC*dI&mUfC*moeG*mA*mU*mG&mG | | and 31B |

d = 2′-H (deoxyribose; DNA); * = phosphorothioate (PS); & = (mesyl) methanesulfonyl; m = 2′-OMe; f = 2′-fluoro; moe = 2′-O-methoxyethyl (2′-MOE); I = inosine; MeC = 5′-methylcytidine.

### Example 15. LNP-Formulated Oligonucleotides Show High Levels of Editing but Limited Durability

To evaluate the efficacy of AI-0991 oligos in a lipid nanoparticle (LNP) formulation, a single intravenous injection (at a 1 mg/kg or 2 mg/kg dose) was administered to 10-week-old NSG-PiZ male or female mice on Day 0. A control group of mice was treated with PBS.

Samples were collected to assess editing efficiency, liver enzyme parameters and pharmacodynamics. Mice were observed for any clinical signs and changes in body weight during the In-life study. Serum was collected pre-dose, on Day 1, on Day 4, and on Day 7 post-treatment. 3 mice per timepoint per condition were sacrificed on Day 1, Day 4, and Day 7 post-treatment, and tissue was collected to measure editing efficiency.

As seen in FIGS. 31A and 31B, LNP-formulated oligonucleotide showed high levels of initial editing and M-AAT production, but a rapid decline in kinetics was observed. GalNAc-conjugated oligonucleotides are expected to show higher durability relative to these LNP formulations.

### Example 16. In Vivo Study in Mice

This study was designed to assess PK and PD of AI-1068 upon four initiation doses (Days 0, 2, 4, and 7) followed by biweekly (every two weeks) maintenance dosing over a period of 8 weeks. Comparison of liver oligonucleotide content in mice sacrificed on Day 7 vs. Day 56 revealed no AI-1068 accumulation due to repeat dosing, consistent with AI-1068 half-life of less than two weeks. Likewise, liver RNA editing levels were comparable between Day 7 and Day 56. Serum M AAT levels were maintained at 20-36 μmol/L over the duration of the study in the 10 mg/kg dosing group, well above the protective threshold of 14 μmol/L. Total AAT levels were maintained at 49-63 μmol/L in the 10 mg/kg group, exceeding the vehicle baseline by up to 4.5-fold. Histology analysis revealed improvements in the liver phenotype of AI-1068-treated animals. These results indicate that long-term repeat dosing of AI 1068 can maintain protective levels of M AAT that may benefit AATD patients.

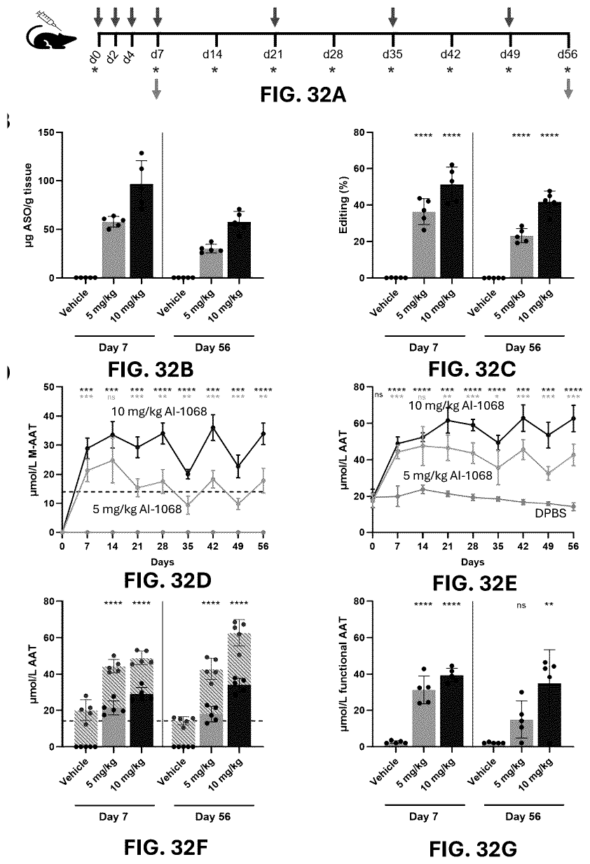

Methods and Materials: AI 1068 was administered by SC injection in 7 week-old male NSG-PiZ mice with four initiation doses (Days 0, 2, 4, and 7) followed by maintenance dosing every two weeks over a total period of 8 weeks (5 mg/kg or 10 mg/kg each dose) (FIG. 32A). Control mice received DPBS injections. Terminal liver samples were collected on Days 7 and 56 to evaluate liver oligonucleotide content and RNA editing levels. Serum was collected weekly to measure AAT levels. Mice were observed weekly for any clinical signs and changes in body weight. All animals survived until their scheduled euthanasia on Day 7 or 56. There were no test article related clinical observations.

Oligonucleotide content: Liver oligonucleotide content was evaluated using a hybridization-based MSD assay. AI-1068 exposure was 100±20 μg/g on Day 7 (3 days after Day 0-4 loading dose, 10 mg/kg) and 58±11 μg/g tissue on Day 56 (7 days after last dose) (FIG. 32B); LLOQ=0.5 μg/g).

Editing: Liver RNA editing was measured on Days 7 and 56 by amplicon sequencing. AI-1068-treated groups showed dose-dependent editing of 36±7% (5 mg/kg) to 51±9% (10 mg/kg) on Day 7 (3 days after last dose) and 23±4% to 42±6% editing on Day 56 (7 days after last dose) (FIG. 32C).

AAT Levels: Serum levels of M-AAT and total AAT were measured weekly by LC-MS/MS. M-AAT levels increased from below LLOQ (0.03 μmol/L) to 21±4 μmol/L (5 mg/kg dose) and 29±4 μmol/L (10 mg/kg dose) by Day 7. Mice treated with 10 mg/kg AI-1068 maintained 20-36 μmol/L M-AAT over the duration of the study, stably remaining above the protective threshold of 14 μmol/L (FIG. 32D). The 5 mg/kg AI-1068 dosing group maintained average levels of 9.4-25 μmol/L serum M-AAT, decreasing slightly below the protective threshold between the biweekly doses. Total serum AAT level rose from a pre-dose level of approximately 20 μmol/L to 44±4 μmol/L (5 mg/kg dose) or 49±4 μmol/L (10 mg/kg dose) by Day 7. Mice treated with 10 mg/kg AI-1068 maintained 49-63 μmol/L total AAT over the duration of the study, exceeding the AAT-level in vehicle-treated group by 4.5-fold at the conclusion of the study on Day 56 (FIG. 32E).

Functional AAT Levels: Serum levels of functional AAT protein were determined by measuring the inhibition of human neutrophil elastase activity (abcam kit ab118971). To obtain functional AAT concentrations, the rates of fluorescent substrate cleavage in mouse serum from Day 7 and Day 56 were interpolated to a standard curve of human AAT in mouse serum matrix. Functional AAT levels showed high concordance with the M-AAT levels measured by LC-MS/MS, indicating that our RNA editing approach successfully restored fully functional AAT protein (FIGS. 32F-32G).

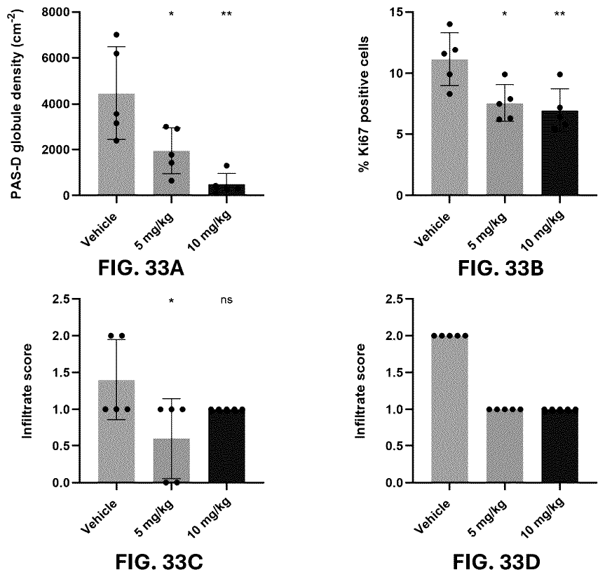

Histology analysis: Analysis of fixed livers from mice dosed with AI-1068 over 8 weeks revealed significant improvements in the liver phenotype compared to vehicle control (FIGS. 33A-33D). There was a substantial reduction in the number of large (≥60 μm2) Z-AAT globules, as visualized with the Periodic Acid-Schiff with Diastase (PAS-D) stain (FIG. 33A). Furthermore, there was substantial suppression of proliferation (marked by Ki-67; FIG. 33B) and immune infiltration (FIGS. 33C-33D) compared to vehicle-treated controls on Day 56. These observations suggest improvement of liver injury upon AI-1068 treatment. Taken together, the data demonstrate that AI-1068 provides a functional benefit by increasing M-AAT and total AAT in serum as well as resolving liver injury caused by mutant Z-AAT protein accumulation.

Serum chemistry: The tolerability of long term treatment in the NSG-PiZ mouse model was also evaluated by limited serum chemistry. AI-1068 treated NSG PiZ mice showed no significant dose dependent increase in liver or kidney toxicity markers above the vehicle control.

Conclusion: Subcutaneous administration of AI-1068 with three initiation doses followed by biweekly maintenance dosing led to durable enhancements in serum M-AAT and total AAT levels. Mice dosed with 10 mg/kg AI-1068 maintained M-AAT levels well above the 14 μmol/L protective threshold for the duration of the study and total AAT levels up to 4.5-fold above baseline. The M-AAT produced upon RNA editing was confirmed to be fully functional by a neutrophil elastase inhibition assay. Biweekly repeat dosing did not result in accumulation of oligonucleotide or edited RNA in the liver, as expected given the approximately 1-week liver half-life of AI-1068. The liver phenotype of AI-1068-treated animals showed significant improvements relative to control group after repeat dosing over eight weeks.

### Example 17. Assessment of AI-2863 Editing in Donor-Derived Primary Human MZ Hepatocytes

To test AI-2863 activity in human hepatocytes, donor-derived human hepatocytes heterozygous for the SERPINA15342K mutation were tested (MZ genotype).

Primary human hepatocytes derived from heterozygous donors carrying the mutation A1ATD E342K (AnaBios, US) were plated into collagen-coated 96-well plates at a density of 70,000 cells per well. Six hours after plating, the medium was changed, and cells were treated with a serial dilution of AI-2863 ranging from 0.001 μM to 10 μM in phosphate-buffered saline (PBS). Twenty-four hours after treatment, cells were lysed, and the RNA was isolated with the Dynabeads mRNA purification kit (Invitrogen), followed by reverse transcription, polymerase chain reaction (PCR), and sequencing of the SERPINA1 amplicon by next-generation sequencing (NGS).

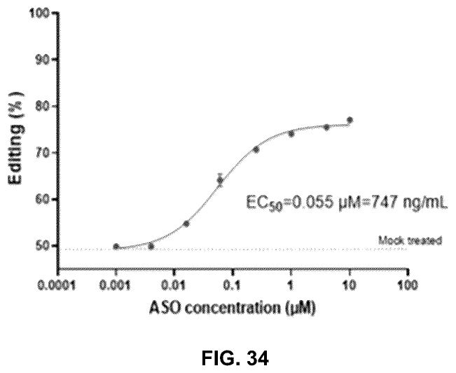

Gymnotic delivery of AI-2863 to patient-derived hepatocytes led to a concentration-dependent increase in SERPINA1 RNA editing with an EC₅₀ of 0.055 μM (95% CI: 0.047-0.064 μM) (FIG. 34). Concentration-dependent RNA editing in MZ hepatocytes following ASO treatment (FIG. 34) provides clear evidence that AI-2863 efficiently engages human ADAR machinery in liver cells.

### Example 18. Assessment of AI-2863 Editing in NGS-PiZ Mice

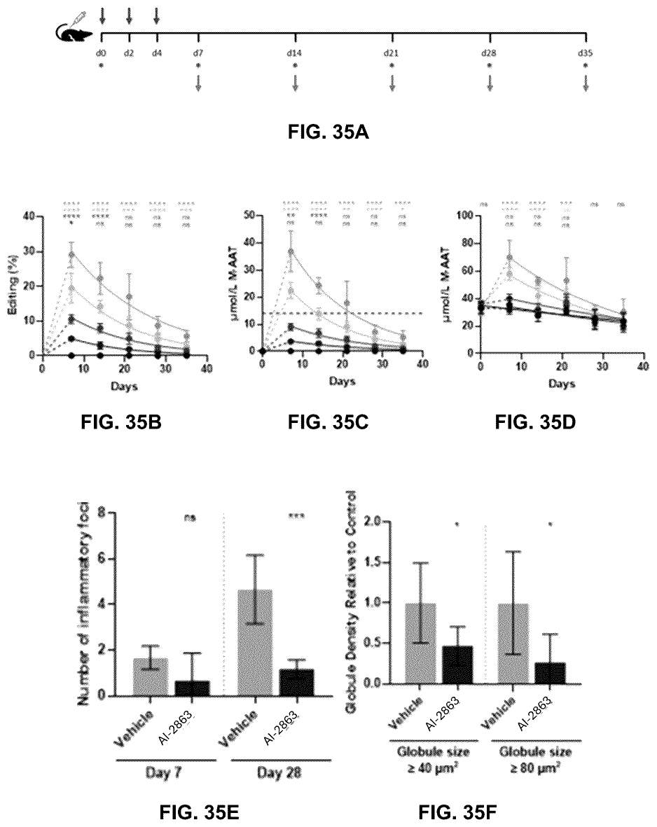

The activity of AI-2863 was assessed in NSG-PiZ mice over the course of 35 days (FIG. 35A). 7 week old male and female NSG PiZ mice were dosed subcutaneously with DPBS (vehicle) or 1-10 mg/kg AI-2863 on Days 0, 2, and 4. Terminal collection of liver and serum was performed on Days 7, 14, 21, 28, and 35. Collection of in-life serum was performed before the first dose and at 0.5, 2, 6, 24 and 48 hours after the last dose.

Liver RNA editing was measured on Days 7, 14, 21, 28, and 35 by next-generation amplicon sequencing. Editing levels increased in a concentration-dependent manner and reached a maximum level of 29±4% on Day 7 (10 mg/kg dose). Significant editing was still detected on Day 35 (5.6±1.7%, 10 mg/kg dose), consistent with prolonged oligonucleotide exposure in the liver and a half-life of approximately 1.5-2 weeks (FIG. 35B).

Serum levels of M-AAT and total AAT protein were measured by liquid chromatography tandem mass spectrometry (LC-MS/MS) on Days 0, 7, 14, 21, 28, and 35. M-AAT showed a dose-dependent increase from below detection to 37±8 μmol/L on Day 7. M-AAT remained at or above the protective level of 14 μmol/L for 3 weeks at the 10-mg/kg dose and 2 weeks at the 5-mg/kg dose (FIG. 35C). Total serum AAT level increased dose dependently from a pre-dose level of 34±4 μmol/L to 70±12 μmol/L AAT on Day 7, returning to vehicle baseline by Day 28 (FIG. 35D).

To evaluate the activity of AI-2863 on liver inflammation, histopathology analysis was performed in NSG-PiZ mice treated with vehicle or the high dose of 10 mg/kg AI-2863. Inflammatory foci were counted in preserved livers from mice at Day 28. On Day 28, the liver was also analyzed by PAS-D staining to visualize Z-AAT globules.

Animals treated with AI-2863 showed significant reduction in the number of inflammatory foci on Day 28 relative to the control group (FIG. 35E). Treatment with 10 mg/kg AI-2863 led to a significant reduction in the density of large PAS-D-stained globules relative to the vehicle control group (FIG. 35F), with up to approx. 3.8-fold reduction in globules larger than 80 μm2. The decrease in liver inflammation and density of large Z-AAT globules indicates the ability of AI-2863 to improve the liver injury phenotype caused by Z-AAT accumulation.

In summary, these data demonstrate that administration of AI-2863 at 5 mg/kg in the transgenic NSG-PiZ mouse model provides a functional benefit by increasing wild-type M AAT and total AAT above the optimal threshold in serum. Effects at this dose level were maintained for more than 2 weeks after the last dose. In addition, administration at 10 mg/kg resulted in significant reduction of the liver aggregates as well as liver inflammation associated with Z-AAT aggregates (other dose levels not evaluated).

### Example 19. Assessment of AI-2863 Editing in NGS-PiZ Mice

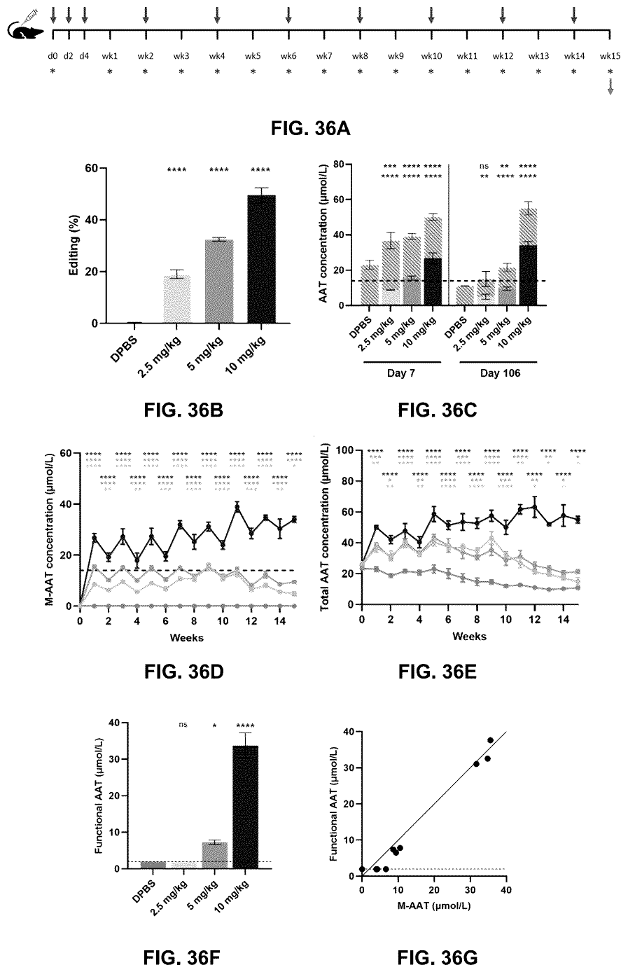

The activity of AI-2863 was assessed by administration of three initiation doses (Days 0, 2, and 4) followed by biweekly (every two weeks) maintenance dosing over a period of 15 weeks (FIG. 36A).

Liver RNA editing was measured on Day 106 by amplicon sequencing. AI-2863-treated groups showed dose-dependent editing of 19.0±1.7% (2.5 mg/kg) to 50±3% (10 mg/kg) (FIG. 36B).

Serum levels of M-AAT and total AAT were measured weekly by LC-MS/MS. M-AAT levels increased from below Lower Limit of Quantification (LLOQ) to 8.67±0.12 μmol/L (2.5 mg/kg dose), 15.5±1.1 μmol/L (5 mg/kg dose), and 27±3 μmol/L (10 mg/kg dose) by Day 7 (FIG. 36C). In the 10 mg/kg dose group (males), serum M-AAT levels were maintained at 18-39 μmol/L over the duration of the study, well above the optimal threshold of 14 μmol/L (FIG. 36D), The M-AAT levels showed clear accumulation over the study period (FIG. 36D), suggesting potential long-term benefits in patients with AATD, Total AAT levels were maintained at 40-63 μmol/L, exceeding the vehicle baseline by 5.1-fold at the end of the study (FIG. 36E).

Serum levels of functional AAT protein were determined by measuring the inhibition of human neutrophil elastase activity. Functional AAT levels showed high concordance with the M-AAT levels measured by LC MS/MS, indicating that the RNA editing successfully restored fully functional AAT protein (FIG. 36F-36G).

The restored M-AAT protein was fully functional, as demonstrated by a neutrophil elastase inhibition assay (FIG. 36G).

### Example 20. Assessment of AI-2863 Single-Dose Editing in NGS-PiZ Mice

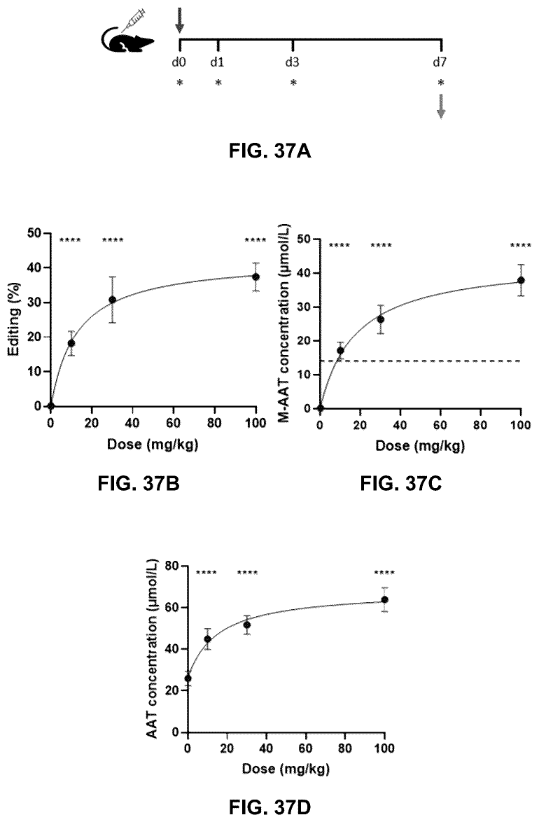

The effects of a single dose (up to 100 mg/kg) of AI-2863 were assessed in the NSG-PiZ mouse model. AI-2863 was administered subcutaneously on Day 0 as a single dose of 10 mg/kg, 30 mg/kg, or 100 mg/kg (FIG. 37A).

Liver RNA editing was measured on Day 7 by amplicon sequencing. Editing levels increased in a dose-dependent manner and reached a maximum level of 37±4% on Day 7 (100 mg/kg dose) (FIG. 37B).

Serum levels of M-AAT and total AAT protein were measured by LC-MS/MS on Day 7. M-AAT showed a dose-dependent increase from below detection to 38±5 μmol/L on Day 7 (FIG. 37C). M-AAT was above the protective level of 14 μmol/L at >10 mg/kg on Day 7 (FIG. 37C). Total serum AAT level increased dose dependently from a pre-dose level of 26±3 μmol/L to 64±6 μmol/L AAT on Day 7 (FIG. 37D).

Serum chemistry and histopathology analysis did not identify any safety concerns.

Those having ordinary skill in the art will appreciate that the disclosure can be modified in ways not specifically described herein.

### REFERENCES

1. Bass, B. L.; Weintraub H. (1987). A developmentally regulated activity that unwinds RNA duplexes. Cell. 48:607-13.
2. Cideciyan, A. V.; Jacobson, S. G.; Drack, A. V.; Ho, A. C.; Charng, J.; Garafalo, A. V.; et al. (2019). Effect of an intravitreal antisense oligonucleotide on vision in Leber congenital amaurosis due to a photoreceptor cilium defect. Nat. Med. 25(2): 225-228.
3. Crooke, S. T.; Vickers, T. A.; and Liang, X. (2020). Phosphorothioate modified oligonucleotide-protein interactions. Nucleic Acids Research. 48(10): 5235-5253.
4. Debacker, A. J.; Voutila, J.; Catley, M.; Blkey, D.; and Habib, N. (2020). Delivery of Oligonucleotides to the Liver with GalNAc: From Research to Registered Therapeutic Drug. Molecular Therapy. 28(8): 1759-1771.
5. Gabriel, C. M.; Pimentel, B. R.; Gomez, C. A.; Cedillo, I.; A. A. Rodriguez. (2022). Improved Purification of GalNAc-Conjugated Antisense Oligonucleotides Using Boronic Acids’; Org. Process Res. Dev. 26:413-421
6. Gagliardi, M., and Ashizawa, A. T. (2021). The Challenges and Strategies of Antisense Oligonucleotide Drug Delivery. Biomedicines. 9(4): 433.
7. Green and Sambrook, Molecular Cloning: A Laboratory Manual (4th ed., Cold Spring Harbor Laboratory Press, Cold Spring Harbor, N.Y. (2012).
8. Iwamoto, N.; Butler, D. C. D.; Svrzikapa, N.; Mohapatra, S.; Zlatev, I.; Sah, D. W. Y.; Meena, Standley, S. M. et al. (2017). Control of phosphorothioate stereochemistry substantially increases the efficacy of antisense oligonucleotides. Nat. Biotech. 35:845-851.
9. Janas, M. M.; Jiang, Y.; Schlegel, M. K.; Waldron, S.; Kuchimanchi, S.; and Barros, S. A . . . (2017). Impact of Oligonucleotide Structure, Chemistry, and Delivery Method on In Vitro Cytotoxicity. Nucleic Acid Therapeutics. 27(1):
10. Levin, A. A. (2019). Treating Disease at the RNA Level with Oligonucleotide. New Engl. J. Med.; 380:57-70.
11. Lorenz, P.; Baker, B. F.; Bennett, C. F.; and Spector, D. L. (1998). Phosphorothioate antisense oligonucleotides induce the formation of nuclear bodies. Mol. Biol. Cell. 9:1007-1023.
12. Merkle, T.; Merz, S.; Reautschnig, P.; Blaha, A.; Li, Q.; Vogel, P.; Wettengel, J; Li, J. B.; Stafforst, T. (2019). Precise RNA editing by recruiting endogenous ADARs with antisense oligonucleotides. Nature Biotechnol. 37:133-138.
13. Quemener, A. M.; Bachelot, L.; Forestier, A.; Donnou-Fournet, E.; Gilot, D.; and Galibert, M.-D. (2019). The powerful world of antisense oligonucleotides: From bench to bedside. Wiley Interdiscip. Rev. RNA. 11(5): e1594.
14. Rebagliati, M. R.; Melton D. A. (1987). Antisense RNA injections in fertilized frog eggs reveal an RNA duplex unwinding activity. Cell. 48:599-605. Zinshteyn, B., and Nishikura, K. (2009). Adenosine-to-inosine RNA editing. Rev. Syst. Biol. Med. 1(2): 202-209.
15. Stephenson, M. L; and Zamecnik, P. C. (1978). Inhibition of Rous sarcoma viral RNA translation by a specific oligodeoxyribonucleotide. Proc. Natl. Acad. Sci. USA. 75(1): 285-288.
16. Thomas, J. M.; and Beal, P. A. (2017). How do ADARs bind RNA? New protein-RNA structures illuminate substrate recognition by the RNA editing ADARs. Bioassays. 39(4): 1-17.
17. Tomaselli, S.; Locatelli, F.; Gallo, A. (2014). The RNA editing enzymes ADARs: mechanism of action and human disease. Cell and Tissue Research. 356:527-532.
18. Vogel, P.; Schneider, M. F.; Wettengel, J.; and Stafforst, T. (2014). Improving site-directed RNA editing in vitro and in cell culture by chemical modification of the guideRNA. Angew. Chem. Int. Ed. Engl. 53 (24): 6267-6271.
19. Wulff, B.-E.; and Nishikura, K. (2010). Substitutional A-to-I RNA editing. Interdiscip. Rev. RNA. 1(1): 90-101.

1.-51. (canceled)

52. A chemically modified oligonucleotide comprising the structure of:

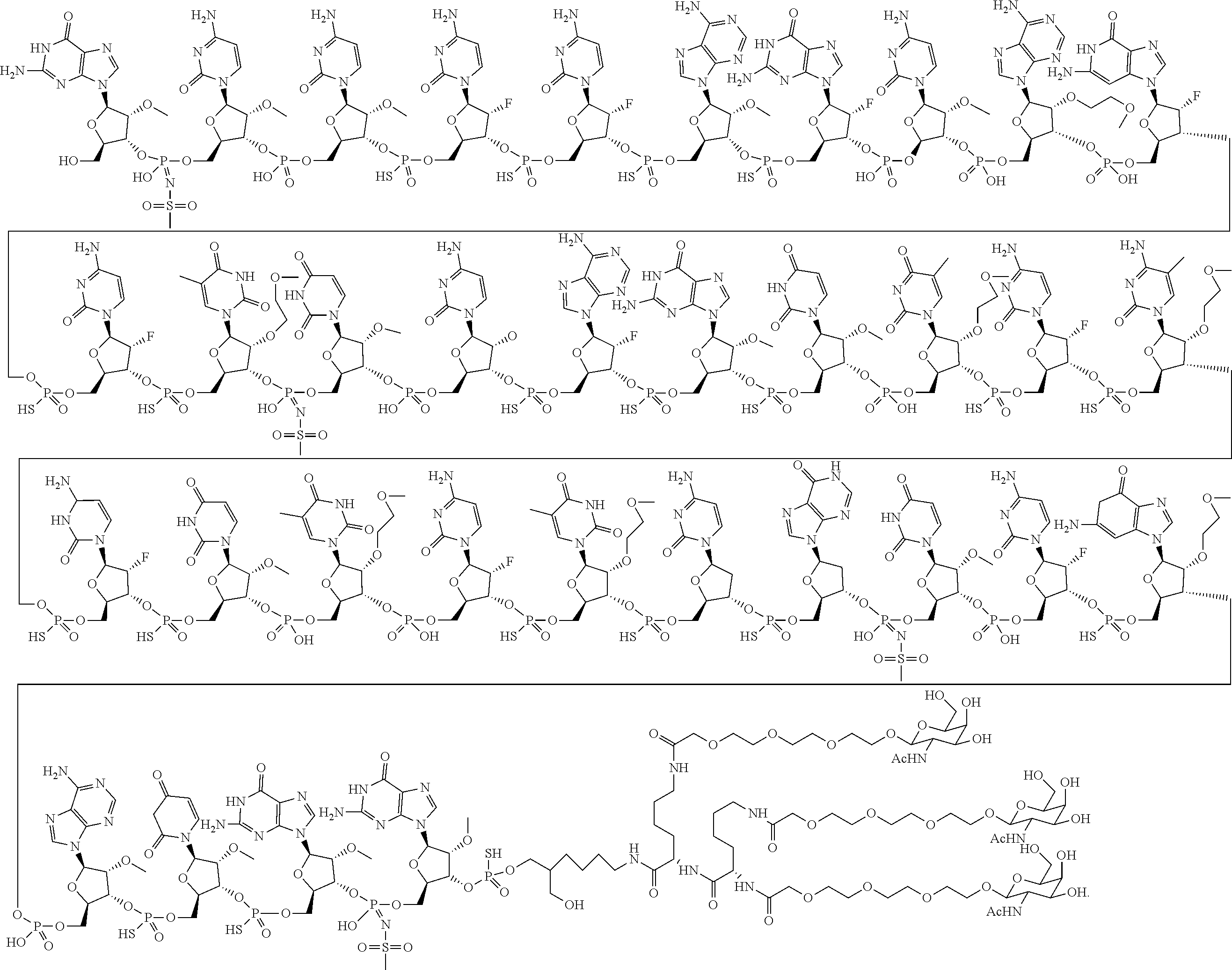

53. A chemically modified oligonucleotide consisting of the structure of:

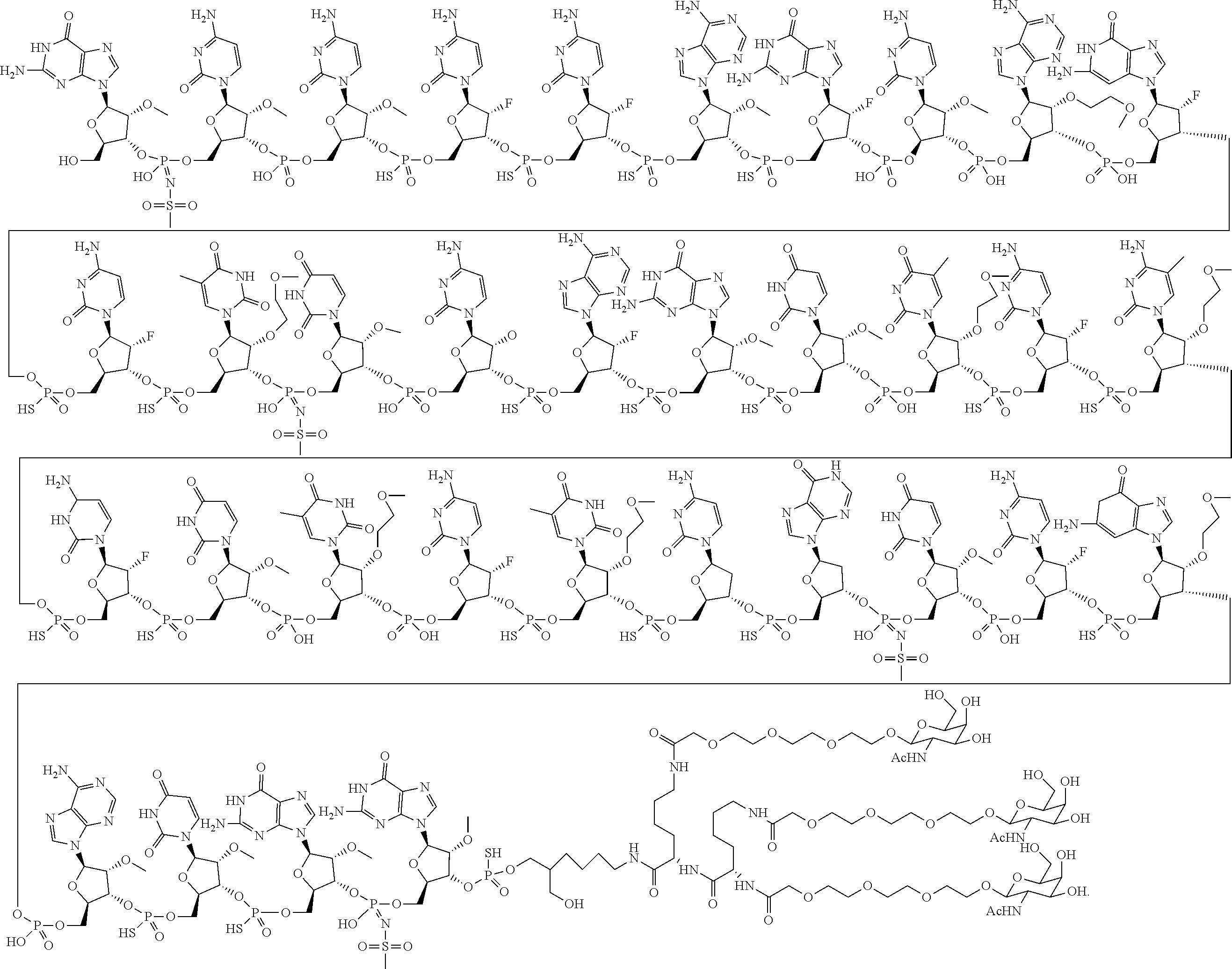

54. A composition comprising the chemically modified oligonucleotide of claim 52, or a pharmaceutically acceptable salt thereof.

55. The composition of claim 54, further comprising a pharmaceutically acceptable carrier.

56. A method comprising administering the chemically modified oligonucleotide of claim 52, or a pharmaceutically acceptable salt thereof, to a subject.

57. The method of claim 56, wherein the administration results in site-directed A-to-I editing of a target adenosine in a SERPINA1 RNA with endogenous adenosine deaminase acting on RNA (ADAR) in the subject, wherein the target adenosine results from a G-to-A mutation in a SERPINA1 gene that causes an E342K substitution in an alpha-1 antitrypsin (AAT) protein encoded by the SERPINA1 RNA.

58. The method of claim 56, wherein the subject has alpha-1 antitrypsin deficiency (A1AD).

59. The method of claim 56, wherein the chemically modified oligonucleotide, or the pharmaceutically acceptable salt thereof, is administered at an effective amount that leads to an increase in M-AAT serum levels and/or total AAT serum levels in the subject.

60. The method of claim 56, wherein the subject is a human.

61. The method of claim 56, wherein the administration is subcutaneous.

62. A method of inducing site-directed A-to-I editing of a target adenosine in a SERPINA1 RNA with endogenous adenosine deaminase acting on RNA (ADAR) in a cell, the method comprising contacting the chemically modified oligonucleotide of claim 52, or the pharmaceutically acceptable salt thereof, with the cell, wherein the target adenosine results from a G-to-A mutation in a SERPINA1 gene that causes an E342K substitution in an alpha-1 antitrypsin (AAT) protein encoded by the SERPINA1 RNA.

63. The method of claim 62, wherein the cell is in vitro.

64. The method of claim 62, wherein the cell is in vivo in a subject.

65. The method of claim 64, wherein the subject has alpha-1 antitrypsin deficiency (A1AD).

66. The method of claim 64, wherein the contacting leads to an increase in M-AAT serum levels and/or total AAT serum levels in the subject.

67. The method of claim 64, wherein the subject is a human.

68. The method of claim 64, wherein the administration is subcutaneous.

69. A method comprising subcutaneously administering a composition comprising the chemically modified oligonucleotide of claim 53, or a pharmaceutically acceptable salt thereof, to a human subject, wherein the human subject has alpha-1 antitrypsin deficiency (A1AD).

70. The method of claim 69, wherein the human subject has a G-to-A mutation in a SERPINA1 gene that causes an E342K substitution in an alpha-1 antitrypsin (AAT) protein encoded by the SERPINA1 RNA.

71. The method of claim 70, wherein the administration induces site-directed A-to-I editing of the adenosine resulting from the G-to-A mutation in the SERPINA1 gene.

72. The method of claim 71, wherein the administration leads to an increase in M-AAT serum levels and/or total AAT serum levels in the subject.


SEQUENCE LISTING (25 sequences)

SEQ ID NO: 1 - RNA, 34 nt, synthetic construct
  source 1..34: mol_type = other RNA
  misc_difference 17: note = n is thymine or uracil
  misc_difference 27: note = n is inosine or guanosine, preferably deoxyinosine or deoxyguanosine
  modified_base order(12,23,25): mod_base = OTHER; note = thymine
gccccagcagcttcagnccctttctcntcgatgg

SEQ ID NO: 2 - RNA, 34 nt, synthetic construct
  source 1..34: mol_type = other RNA
  modified_base order(12,23,25): mod_base = OTHER; note = thymine
  modified_base 27: mod_base = i
gccccagcagcttcagtccctttctcntcgatgg

SEQ ID NO: 3 - RNA, 34 nt, synthetic construct; identical to SEQ ID NO: 2
  source 1..34: mol_type = other RNA
  modified_base order(13,23,25): mod_base = OTHER; note = thymine
  modified_base 27: mod_base = i

SEQ ID NO: 4 - DNA, 34 bp, synthetic construct
  source 1..34: mol_type = other DNA
  modified_base order(1..3,6,8,14,16,22,28,31..34): mod_base = OTHER; note = 2prime-O-methyl nucleosides
  modified_base order(9,12,18,20,23,25,30): mod_base = OTHER; note = 2prime-O-methoxyethyl
  modified_base order(1..2,4..5,12..13,21..22,27..28,33..34): mod_base = OTHER; note = methanesulfonyl linkage
  modified_base order(4,5,7,10,11,13,15,17,19,21,24,29): mod_base = OTHER; note = 2prime-Fluoro-nucleosides
  modified_base order(2..3,5..8,10..12,13..17,18..20,24..27,29..33): mod_base = OTHER; note = phosphorothioate linkage
  modified_base order(1..2,4..5,12..13,21..22,27..28,33..34): mod_base = OTHER; note = methanesulfonyl linkage
  modified_base order(18,20): mod_base = OTHER; note = 5prime-methylcytidine
  modified_base order(26,27): mod_base = OTHER; note = 2prime-H deoxyribose
  modified_base 27: mod_base = i
  modified_base order(12,23,25): mod_base = OTHER; note = Thymine
  misc_feature order(26,27): note = DNA
  misc_feature order(1..25,28..34): note = RNA
gccccagcagcttcagtccctttctcntcgatgg

SEQ ID NO: 5 - DNA, 34 bp, synthetic construct; identical to SEQ ID NO: 4
  source 1..34: mol_type = other DNA
  modified_base order(1..3,6,8,14,16,22,28,31..34): mod_base = OTHER; note = 2prime-O-methyl nucleoside
  modified_base order(1..2,27..28,33..34): mod_base = OTHER; note = methanesulfonyl linkage
  modified_base 27: mod_base = i
  modified_base order(3..8,10..12,13..16,19..20,21..22,24..27,28..30,32..33): mod_base = OTHER; note = phosphorothioate linkage
  modified_base order(9,12,17,18,20,23,25,30): mod_base = OTHER; note = 2prime-O-methoxyethyl
  modified_base order(4,5,7,10,11,13,15,19,21,24,29): mod_base = OTHER; note = 2prime-Fluoro-nucleosides
  modified_base order(26,27): mod_base = OTHER; note = 2prime-H deoxyribose
  modified_base order(18,20): mod_base = OTHER; note = 5prime-methylcytidine
  modified_base order(12,17,23,25): mod_base = OTHER; note = Thymine
  misc_feature order(26,27): note = DNA
  misc_feature order(1..25,28..34): note = RNA

SEQ ID NO: 6 - DNA, 34 bp, synthetic construct; identical to SEQ ID NO: 4
  source 1..34: mol_type = other DNA
  modified_base order(1..2,6,8,16..17,22,28,31..34): mod_base = OTHER; note = 2prime-O-methyl nucleosides
  modified_base order(1..2,12..13,27..28,33..34): mod_base = OTHER; note = methanesulfonyl linkage
  modified_base order(4..8,10..12,13..14,15..17,19..20,21..22,24..27,28..30,31..33): mod_base = OTHER; note = phosphorothioate linkage
  modified_base order(3,9,12,14,18,20,23,25,30): mod_base = OTHER; note = 2prime-O-methoxyethyl
  modified_base order(4..5,7,10..11,13,15,19,21,24,29): mod_base = OTHER; note = 2prime-Fluoro-nucleosides
  modified_base order(3,13,16,17): mod_base = OTHER; note = 5prime-methylcytidine
  modified_base 27: mod_base = i
  modified_base order(26,27): mod_base = OTHER; note = 2prime-H deoxyribose
  modified_base order(12,23,25): mod_base = OTHER; note = Thymine
  misc_structure order(26,27): note = DNA
  misc_structure order(1..25,28..34): note = RNA

SEQ ID NO: 7 - DNA, 34 bp, synthetic construct; identical to SEQ ID NO: 4
  source 1..34: mol_type = other DNA
  modified_base order(1..3,6,8,13,14,16..17,22,28,31..34): mod_base = OTHER; note = 2prime-O-methyl nucleosides
  modified_base order(1..2,12..13,27..28,33..34): mod_base = OTHER; note = methanesulfonyl linkage
  modified_base order(3..8,10..12,14..17,18..20,21..22,24..27,29..30,31..34): mod_base = OTHER; note = phosphorothioate linkage
  modified_base order(9,12,18,20,23,25,30): mod_base = OTHER; note = 2prime-O-methoxyethyl
  modified_base order(4,5,7,10,11,15,19,21,24,29): mod_base = OTHER; note = 2prime-Fluoro-nucleosides
  modified_base order(18,20): mod_base = OTHER; note = 5prime-methylcytidine
  modified_base 27: mod_base = i
  modified_base order(26,27): mod_base = OTHER; note = 2prime-H deoxyribose
  modified_base order(12,23,25): mod_base = OTHER; note = Thymine
  misc_feature order(26,27): note = DNA
  misc_feature order(1..25,28..34): note = RNA

SEQ ID NO: 8 - DNA, 34 bp, synthetic construct; identical to SEQ ID NO: 4
  source 1..34: mol_type = other DNA
  modified_base order(1..3,6,8,13..14,16..17,22,28,32..34): mod_base = OTHER; note = 2prime-O-methyl nucleosides
  modified_base order(1..2,12..13,27..28,31..32,33..34): mod_base = OTHER; note = methanesulfonyl linkage
  modified_base order(3..8,10..12,14..17,18..20,21..22,24..27,29..30,32..33): mod_base = OTHER; note = phosphorothioate linkage
  modified_base order(9,12,18,20,23,25,30): mod_base = OTHER; note = 2prime-O-methoxyethyl
  modified_base order(4,5,7,10,11,15,21,24,29,31): mod_base = OTHER; note = 2prime-Fluoro-nucleosides
  modified_base order(18,20): mod_base = OTHER; note = 5prime-methylcytidine
  modified_base 27: mod_base = i
  modified_base order(26,27): mod_base = OTHER; note = 2prime-H deoxyribose
  modified_base order(12,23,25): mod_base = OTHER; note = Thymine
  misc_feature order(26,27): note = DNA
  misc_feature order(1..25,28..34): note = RNA

SEQ ID NO: 9 - DNA, 34 bp, synthetic construct; identical to SEQ ID NO: 4
  source 1..34: mol_type = other DNA
  modified_base order(1..3,6,8,13..14,16..17,20,26,30..32): mod_base = OTHER; note = 2prime-O-methyl nucleosides
  modified_base order(1..2,12..13,27..28,33..34): mod_base = OTHER; note = methanesulfonyl linkage
  modified_base order(3..8,10..12,14..17,18..20,21..22,24..27,29..30,31..33): mod_base = OTHER; note = phosphorothioate linkage
  modified_base order(9,12,18,20,23,25,30): mod_base = OTHER; note = 2prime-O-methoxyethyl
  modified_base order(4,5,7,10,11,15,19,21,24,29,31): mod_base = OTHER; note = 2prime-Fluoro-nucleosides
  modified_base order(18,20): mod_base = OTHER; note = 5prime-methylcytidine
  modified_base 27: mod_base = i
  modified_base order(26,27): mod_base = OTHER; note = 2prime-H deoxyribose
  modified_base order(12,23,25): mod_base = OTHER; note = Thymine
  misc_feature order(26,27): note = DNA
  misc_feature order(1..25,28..34): note = RNA

SEQ ID NO: 10 - DNA, 34 bp, synthetic construct; identical to SEQ ID NO: 4
  source 1..34: mol_type = other DNA
  modified_base order(1..3,6,8,13..14,16..17,22,28,32..34): mod_base = OTHER; note = 2prime-O-methyl nucleosides
  modified_base order(1..2,12..13,27..28,33..34): mod_base = OTHER; note = methanesulfonyl linkage
  modified_base order(3..8,10..12,14..27,18..20,21..22,24..27,28..30,31..33): mod_base = OTHER; note = phosphorothioate linkage
  modified_base order(9,12,18,20,23,25,30): mod_base = OTHER; note = 2prime-O-methoxyethyl
  modified_base order(4,5,7,10,11,15,19,21,24,29,31): mod_base = OTHER; note = 2prime-Fluoro-nucleosides
  modified_base order(18,19): mod_base = OTHER; note = 5prime-methylcytidine
  modified_base 27: mod_base = i
  modified_base order(26,27): mod_base = OTHER; note = 2prime-H deoxyribose
  modified_base order(12,23,25): mod_base = OTHER; note = Thymine
  misc_feature order(26,27): note = DNA
  misc_feature order(1..25,28..34): note = RNA

SEQ ID NO: 11 - DNA, 34 bp, synthetic construct; identical to SEQ ID NO: 4
  source 1..34: mol_type = other DNA
  modified_base order(1..2,6,8,13,16,17,22,28,32..33): mod_base = OTHER; note = 2prime-O-methyl nucleosides
  modified_base order(1..2,12..13,27..28,33..34): mod_base = OTHER; note = methanesulfonyl linkage
  modified_base order(3..8,10..12,14..17,18..20,21..22,23..26,27..29,31..33): mod_base = OTHER; note = phosphorothioate linkage
  modified_base order(3,9,12,14,18,20,23,25,30,34): mod_base = OTHER; note = 2prime-O-methoxyethyl
  modified_base order(4..5,7,10..11,15,19,21,24,29,31): mod_base = OTHER; note = 2prime-Fluoro-nucleosides
  modified_base order(3,14,18,20): mod_base = OTHER; note = 5prime-methylcytidine
  modified_base 27: mod_base = i
  modified_base order(26,27): mod_base = OTHER; note = d = 2prime-H deoxyribose
  modified_base order(12,23,25): mod_base = OTHER; note = Thymine
  misc_feature order(26,27): note = DNA
  misc_feature order(1..25,28..34): note = RNA

SEQ ID NO: 12 - DNA, 34 bp, synthetic construct; identical to SEQ ID NO: 4
  source 1..34: mol_type = other DNA
  modified_base order(1..3,6,8,14,16,22,28,31..34): mod_base = OTHER; note = 2prime-O-methyl nucleosides
  modified_base order(26,27): mod_base = OTHER; note = d is 2prime-H deoxyribose
  modified_base order(2..3,5..8,10..12,13..14,15..17,18..20,24..27,29..33): mod_base = OTHER; note = phosphorothioate linkage
  modified_base order(1..2,4..5,12..13,21..22,27..28,33..34): mod_base = OTHER; note = methanesulfonyl linkage
  modified_base order(4,5,7,10..11,13,15,17,19,21,24,29): mod_base = OTHER; note = 2prime-Fluoro-nucleosides
  modified_base order(9,12,18,20,23,25,30): mod_base = OTHER; note = 2prime-O-methoxyethyl
  modified_base 27: mod_base = i
  modified_base order(18,20): mod_base = OTHER; note = 5prime-methylcytidine
  modified_base order(12,23,25): mod_base = OTHER; note = Thymine
  misc_feature order(26,27): note = DNA
  misc_feature order(1..25,28..34): note = RNA

SEQ ID NO: 13 - DNA, 16 bp, synthetic construct
  source 1..16: mol_type = other DNA
  modified_base 4..13: mod_base = OTHER; note = 2prime-H deoxyribose
  modified_base 1..16: mod_base = OTHER; note = phosphorothioate linkage
  modified_base order(1..3,14..16): mod_base = OTHER; note = locked nucleic acid
  modified_base order(4,10..11,14,15,16): mod_base = OTHER; note = Thymine
  modified_base order(2,3): mod_base = OTHER; note = 5prime-methylcytidine
  misc_feature 4..13: note = DNA
  misc_feature order(1..3,14..16): note = RNA
gcctcccagttccttt

SEQ ID NO: 14 - DNA, 32 bp, synthetic construct
  source 1..32: mol_type = other DNA
  modified_base order(1,4,6,11..12,14,15,20,26,29..32): mod_base = OTHER; note = 2prime-O-methyl nucleosides
  modified_base order(1..2,10..11,25..26,31..32): mod_base = OTHER; note = methanesulfonyl linkage
  modified_base order(2..6,8..10,12..15,16..18,19..20,22..25,27..28,29..31): mod_base = OTHER; note = phosphorothioate linkage
  modified_base order(24,25): mod_base = OTHER; note = 2prime-H deoxyribose
  modified_base order(2..3,5,8..9,13,17,19,22,27): mod_base = OTHER; note = 2prime-Fluoro-nucleosides
  modified_base order(7,10,16,18,21,23,28): mod_base = OTHER; note = 2prime-O-methoxyethyl
  modified_base 25: mod_base = i
  modified_base order(16,18): mod_base = OTHER; note = 5prime-methylcytidine
  modified_base order(10,21,23): mod_base = OTHER; note = Thymine
  misc_feature order(24,25): note = DNA
  misc_feature order(1..23,26..32): note = RNA
cccagcagcttcagtccctttctcntcgatgg

SEQ ID NO: 15 - DNA, 53 bp, synthetic construct
  source 1..53: mol_type = other DNA
tcgtcggcagcgtcagatgtgtataagagacagcacccctgaagctctccaag

SEQ ID NO: 16 - DNA, 56 bp, synthetic construct
  source 1..56: mol_type = other DNA
gtctcgtgggctcggagatgtgtataagagacagggatagacatgggtatggcctc

SEQ ID NO: 17 - DNA, 57 bp, synthetic construct
  source 1..57: mol_type = other DNA
gcataaggctgtgctgaccatcgacccgaaagggactgaagctgctggggccatgaa

SEQ ID NO: 18 - DNA, 57 bp, synthetic construct
  source 1..57: mol_type = other DNA
tcgtcggcagcgtcagatgtgtataagagacaggcatcactaaggtcttcagcaatg

SEQ ID NO: 19 - DNA, 56 bp, synthetic construct; identical to SEQ ID NO: 16
  source 1..56: mol_type = other DNA

SEQ ID NO: 20 - DNA, 37 bp, synthetic construct
  source 1..37: mol_type = other DNA
agctctccccggccgtgcataaggctgtgctgacata

SEQ ID NO: 21 - DNA, 34 bp, synthetic construct; identical to SEQ ID NO: 4
  source 1..34: mol_type = other DNA
  modified_base order(18,20): mod_base = OTHER; note = 5prime-methylcytidine
  modified_base order(26,27): mod_base = OTHER; note = 2prime-H deoxyribose
  modified_base 27: mod_base = i
  modified_base order(12,23,25): mod_base = OTHER; note = Thymine
  modified_base order(4,5,7,10,11,15,19,21,24,29): mod_base = OTHER; note = 2prime-Fluoro-nucleosides
  modified_base order(9,12,18,20,23,25,30): mod_base = OTHER; note = 2prime-O-methoxyethyl
  modified_base order(3..8,10..12,14..17,18..20,21..22,24..27,29..30,31..33): mod_base = OTHER; note = phosphorothioate linkage
  modified_base order(1..2,12..13,27..28,33..34): mod_base = OTHER; note = ethanesulfonyl linkage
  modified_base order(1..3,6,8,13..14,16..17,22,28,31..34): mod_base = OTHER; note = 2prime-O-methyl nucleosides
  misc_structure order(26,27): note = DNA
  misc_feature order(1..25,28..34): note = RNA

SEQ ID NO: 22 - DNA, 34 bp, synthetic construct; identical to SEQ ID NO: 4
  source 1..34: mol_type = other DNA
  modified_base order(18,20): mod_base = OTHER; note = 5prime-methylcytidine
  modified_base order(26,27): mod_base = OTHER; note = 2prime-H deoxyribose
  modified_base 27: mod_base = i
  modified_base order(12,23,25): mod_base = OTHER; note = Thymine
  modified_base order(4..5,7,10..11,15,19,21,24,25): mod_base = OTHER; note = 2prime-Fluoro-nucleosides
  modified_base order(9,12,18,20,23,25,30): mod_base = OTHER; note = 2prime-O-methoxyethyl
  modified_base order(3..8,10..12,14..17,18..20,21..22,24..27,29..30,31..33): mod_base = OTHER; note = phosphorothioate linkage
  modified_base order(1..2,12..13,27..28,33..34): mod_base = OTHER; note = prosyl linkage
  modified_base order(1..3,6,8,13,14,16..17,22,28,31..34): mod_base = OTHER; note = 2prime-O-methyl nucleosides
  misc_structure order(26,27): note = DNA
  misc_feature order(1..25,28..34): note = RNA

SEQ ID NO: 23 - DNA, 34 bp, synthetic construct; identical to SEQ ID NO: 4
  source 1..34: mol_type = other DNA
  modified_base order(18,20): mod_base = OTHER; note = 5prime-methylcytidine
  modified_base 27: mod_base = i
  modified_base order(26,27): mod_base = OTHER; note = 2prime-H deoxyribose
  modified_base order(12,23,25): mod_base = OTHER; note = Thymine
  modified_base order(4..5,7,10,11,15,19,21,24,29): mod_base = OTHER; note = 2prime-Fluoro-nucleosides
  modified_base order(9,12,18,20,23,25,30): mod_base = OTHER; note = 2prime-O-methoxyethyl
  modified_base order(3..8,10..12,14..17,18..20,21..22,24..27,29..30,31..33): mod_base = OTHER; note = phosphorothioate linkage
  modified_base order(1..2,12..13,27..28,33..34): mod_base = OTHER; note = busyl linkage
  modified_base order(1..3,6,8,13..14,16..17,22,28,31..34): mod_base = OTHER; note = 2prime-O-methyl nucleosides
  misc_feature order(26,27): note = DNA
  misc_feature order(1..25,28..34): note = RNA

SEQ ID NO: 24 - DNA, 34 bp, synthetic construct; identical to SEQ ID NO: 4
  source 1..34: mol_type = other DNA
  modified_base order(18,20): mod_base = OTHER; note = 5prime-methylcytidine
  modified_base order(26,27): mod_base = OTHER; note = 2prime-H deoxyribose
  modified_base order(12,23,25): mod_base = OTHER; note = Thymine
  modified_base order(4..5,10,11,15,19,21,24,29): mod_base = OTHER; note = 2prime-Fluoro-nucleosides
  modified_base order(9,12,18,20,23,25,30): mod_base = OTHER; note = 2prime-O-methoxyethyl
  modified_base order(3..8,10..12,14..17,18..22,24..27,29..30,31..33): mod_base = OTHER; note = phosphorothioate linkage
  modified_base order(1..2,12..13,27..28,33..34): mod_base = OTHER; note = hesyl linkage
  modified_base order(1..3,6,8,13..14,16..17,22,28,31..34): mod_base = OTHER; note = 2prime-O-methyl nucleosides
  misc_feature order(26,27): note = DNA
  misc_feature order(1..25,28..34): note = RNA

SEQ ID NO: 25 - DNA, 34 bp, synthetic construct; identical to SEQ ID NO: 4
  source 1..34: mol_type = other DNA
  modified_base order(18,20): mod_base = OTHER; note = 5prime-methylcytidine
  modified_base 27: mod_base = i
  modified_base order(12,23,25): mod_base = OTHER; note = Thymine
  modified_base order(4..5,7,10..11,13,15,17,19,21,24,29): mod_base = OTHER; note = 2prime-Fluoro-nucleosides
  modified_base order(9,12,18,20,23,25,30): mod_base = OTHER; note = 2prime-O-methoxyethyl
  modified_base order(1..3,6,8,14,16,22,28,31..34): mod_base = OTHER; note = 2prime-O-methyl nucleosides
  modified_base order(1..2,4..5,12..13,21..22,27..28,33..34): mod_base = OTHER; note = methanesulfonyl linkage
  modified_base order(2..3,5..8,10..12,13..17,18..20,24..27,29..33): mod_base = OTHER; note = phosphorothioate linkage
  modified_base order(26,27): mod_base = OTHER; note = 2prime-H deoxyribose
  misc_feature order(26,27): note = DNA
  misc_feature order(1..25,28..34): note = RNA